(19) United States
(12) Patent Application Publication
(10) Pub. No.: US 2024/0425513 A1
(43) Pub. Date: Dec. 26, 2024

(54) OXAZOLE, OXADIAZOLE, AND INDOLE DERIVATIVES FOR THE INHIBITION OF USP28

(71) Applicant: Carmot Therapeutics, Inc., Berkeley, CA (US)

(72) Inventors: Xiaohui Du, Berkeley, CA (US); Ray Fucini, Berkeley, CA (US); Jeff lwig, Berkeley, CA (US); Hiroyuki Kawai, Berkeley, CA (US); Joon Won Jeong, Berkeley, CA (US); Daniel Erlanson, Berkeley, CA (US); Stig Hansen, Berkeley, CA (US)

(21) Appl. No.: 18/703,124

(22) PCT Filed: Oct. 21, 2022
(86) PCT No.: PCT/US2022/047438

§ 371 Date: Apr. 19, 2024

Related U.S. Application Data
(60) Provisional application No. 63/271,097, filed on Oct. 22, 2021.

(51) Int. Cl. C07D 487/08; A61K 31/422; A61K 31/4245; A61K 31/4439; A61K 31/444; A61K 31/4545; A61K 31/496; A61K 31/497; A61K 31/501; A61K 31/506; A61K 31/5377; A61K 31/55; C07D 263/32; C07D 271/06; C07D 413/12; C07D 413/14

(52) U.S. Cl. CPC C07D 487/08; A61K 31/422; A61K 31/4245; A61K 31/4439; A61K 31/444; A61K 31/4545; A61K 31/496; A61K 31/497; A61K 31/501; A61K 31/506; A61K 31/5377; A61K 31/55; C07D 263/32; C07D 271/06; C07D 413/12; C07D 413/14

(57) ABSTRACT

Provided herein are USP28 inhibitors, pharmaceutical compositions, methods of their preparation, and methods of their use in treatment and/or diagnosis.

This application claims priority to and the benefit of U.S. Ser. No. 63/271,097 filed Oct. 22, 2021, the entirety of which is hereby incorporated by reference for all purposes.

### FIELD

Provided herein are compounds and pharmaceutical compositions for the inhibition of the deubiquitinating enzyme ubiquitin specific peptidase 28 (USP28). The compounds and compositions are useful, for instance, in methods of treatment, and prevention of diseases or conditions, including, but not limited to, cancer, inflammation, autoimmune diseases, and infectious diseases.

### BACKGROUND

An important post-translation modification is ubiquitination, the process of attaching a target protein with ubiquitin. This is accomplished through a covalent bond between the C-terminus of ubiquitin and a lysine residue of the target protein. Once a protein is tagged with ubiquitin, the protein is then commonly marked for degradation. Recent studies have also shown that ubiquitination assists in coordinating the cellular localization of proteins, activating and inactivating proteins, and/or modulating protein-protein interactions.

Deubiquitinating enzymes (DUBs) play an important role in modulating the ubiquitination process by cleaving the covalent bond between ubiquitin and the target protein to reverse ubiquitination. Approximately 100 human DUBs have been identified and these are divided into two main classes: cysteine proteases and metalloproteases. Of the cysteine proteases, the largest family is the ubiquitin-specific protease (USP/UBP) family that consists of over fifty proteases, including ubiquitin specific peptidase 28 (USP28).

USP28 was initially identified in a homology search of USP25 (Valero, et al. Genome Biol. 2001; 2(10): research0043.1-research0043.10). Like USP25, USP28 contains an ubiquitin-associated domain and ubiquitin interacting motifs in the N-terminal region. Since its identification, studies have shown that USP28 is involved in the promotion of oncoprotein stability, the downregulation of apoptosis, the upregulation of angiogenesis and metastasis, and the maintenance of cell cycle arrest and DNA repair (Wang et al. Cell Death and Disease 2018; 9: 186). For this reason, USP28 has been implicated in many diseases and disorders, including cancer, autoimmune diseases, inflammation, and infectious diseases.

For example, USP28 has been shown to be upregulated in non-small cell lung cancer and high levels of USP28 have been associated with poor prognosis in the disease. (Zhang et al., J. Cell. Mol. Med. 2015; 19:799). USP28 is often also overexpressed in colon cancer (Diefenbacher et al., J. Clin. Invest. 2014; 124:3407) and bladder cancer (Guo et al. Tumour Biol. 2014; 35: 4017). In breast cancer, USP28 is a deubiquitinase of LSD1, an epigenetic regulator. Studies have shown that the disruption of USP28 can lead to the destabilization of LSD1, which in turn can inhibit tumorigenicity (Cao et al. Oncogene 2017; 36: 133).

Deubiquitinating enzymes, including USP25, are also involved in inflammation. For example, USP25 negatively regulates interleukin 17 (IL-17), a pro-inflammatory protein. Overexpression of USP25 also inhibited IL-17-triggered signaling (Zhong et al. Nat Immunol. 2012; 13:1110-7).

There is a need for additional therapies to inhibit USP28 and to treat diseases that are due to USP28 overexpression and/or dysfunction, including cancer, autoimmune diseases, inflammation, and infectious diseases. Small molecules that targeting USP28 should provide safe, stable, and easy to administer therapeutics.

### SUMMARY

The present disclosure includes compounds of Formulas (I), (Ia), (Tb), (Ic), (Id), (II), (IIa), (IIb), (IIc), (IId), and (III) or pharmaceutically acceptable salts, diastereomers, or stereoisomers thereof. Further provided herein are sub-formulas of Formulas (I), (Ia), (Tb), (Ic), (Id), (II), (IIa), (IIb), (IIc), (IId), and (III), compositions comprising the compounds of Formulas (I), (Ia), (Tb), (Ic), (Id), (II), (IIa), (IIb), (IIc), (IId), and (III), and methods of producing the compounds. The compounds of Formulas (I), (Ia), (Tb), (Ic), (Id), (II), (IIa), (IIb), (IIc), (IId), and (III), and sub-formulas and embodiments thereof, are useful for the inhibition of ubiquitin specific peptidase 28 (USP28). In certain embodiments, the compounds can be used for treating diseases, or conditions wherein inhibition of USP28 provides therapeutic benefit, including cancer, autoimmune diseases, inflammation, and infectious diseases.

In one aspect, a compound of Formula (I) or (II) or a pharmaceutically acceptable salt, diastereomer, or stereoisomer thereof is provided:

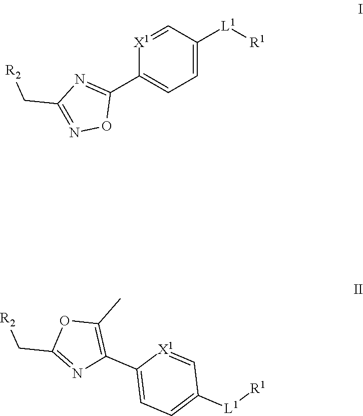

wherein
L¹ is selected from

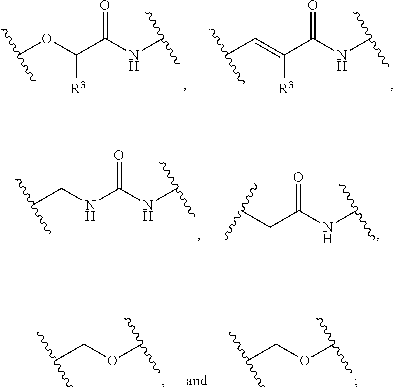

R¹ is selected from aryl substituted with 1, 2, 3, or 4 R^{4a} groups; cycloalkyl substituted with 1, 2, 3, or 4 R^{4a} groups; heteroaryl substituted with 1, 2, 3, or 4 R^{4a} groups; and, heterocycle substituted with 1, 2, 3, or 4 R^{4a} groups wherein the heteroaryl and heterocycle contain at least one nitrogen, oxygen, or sulfur and wherein the nitrogen of the heterocycle is substituted with R^{4b};
R² is selected from aryl optionally substituted with 1, 2, 3, or 4 R⁵ groups; heteroaryl optionally substituted with 1, 2, 3, or 4 R⁵ groups; cycloalkyl optionally substituted with 1, 2, 3, or 4 R⁵ groups; and heterocycle optionally substituted with 1, 2, 3, or 4 R⁵ groups;
R³ is hydrogen or C₁₋₆alkyl;
each R^{4a} is independently selected from C₁₋₆alkyl, C₂₋₆alkenyl, C₂₋₆alkynyl, C₃₋₆cycloalkyl, C₁₋₆haloalkyl, C₁₋₆alkoxy, hydroxyC₁₋₆alkyl, aminoC₁₋₆alkyl, C₁₋₆alkylaminoalkyl, C₁₋₆dialkylaminoalkyl, amino, hydroxy, cyano, nitro, halogen, —NR⁶R⁷, —CH₂NR⁶R⁷, —C(O)NR⁶R⁷, —CH₂C(O)NR⁶R⁷, —(CH₂)ₐ—O—(CH₂)_{b}R⁸, and —C(O)R⁹;
each R^{4b} is independently selected from hydrogen, C₁₋₆alkyl, C₃₋₆cycloalkyl, C₁₋₆haloalkyl, cyano, —CH₂NR⁶R⁷, —C(O)NR⁶R⁷, —CH₂C(O)NR⁶R⁷, —(CH₂)ₐ—O—(CH₂)_{b}R⁸, and —C(O)R⁹;
each R⁵, when present, is independently selected from C₁₋₆alkyl, C₂₋₆alkenyl, C₂₋₆alkynyl, C₃₋₆cycloalkyl, C₁₋₆haloalkyl, C₁₋₆alkoxy, hydroxyC₁₋₆alkyl, aminoC₁₋₆alkyl, C₁₋₆alkylaminoalkyl, C₁₋₆dialkylaminoalkyl, amino, hydroxy, cyano, nitro, halogen, —NR⁶R⁷, —CH₂NR⁶R⁷, —C(O)NR⁶R⁷, —CH₂C(O)NR⁶R⁷, —(CH₂)ₐ—O—(CH₂)_{b}R⁸, and —C(O)R⁹;
R⁶ and R⁷ are independently selected from hydrogen, C₁₋₆alkyl, C₃₋₆cycloalkyl, aryl, arylC₁₋₆alkyl, heteroaryl, heteroarylC₁₋₆alkyl, heterocycle, and heterocycloC₁₋₆alkyl, wherein R⁶ and R⁷, with the exception of hydrogen, can independently be optionally substituted with 1 or 2 R¹⁰ groups;
or R⁶ and R⁷ are joined together to form a heterocycle or a biheterocycle optionally substituted with 1 or 2 R¹⁰ groups;
R⁸ is hydroxy, cyano, halogen, C₁₋₆haloalkyl, —NR⁶R⁷, or —C(O)R⁹.
R⁹ is C₁₋₆alkyl, hydroxy, C₁₋₆alkoxy, aryl, aryloxy, arylC₁₋₆alkyl, aryloxyC₁₋₆alkyl, heteroaryl, heteroarylC₁₋₆alkyl, heterocycle, heterocycloC₁₋₆alkyl, or —NR¹¹R¹²;
R¹⁰ is independently selected from —C(O)R⁹, —COOH, amino, —NR¹¹R¹², —NR¹¹C(O)R¹², aryl, heteroaryl, arylC₁₋₆alkyl, and heteroarylC₁₋₆alkyl;
or 2 R¹⁰ groups, when on the same carbon, can be taken together to form an oxo group;
R¹¹ and R¹² are independently selected from hydrogen and C₁₋₆alkyl;
X¹ is CH or N; and
a and be are integers independently selected from 1, 2, 3, and 4.

In one aspect, a compound of Formula (III) or a pharmaceutically acceptable salt, diastereomer, or stereoisomer thereof is provided:

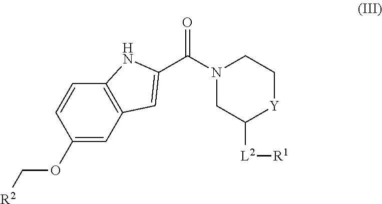

wherein
L² is selected from

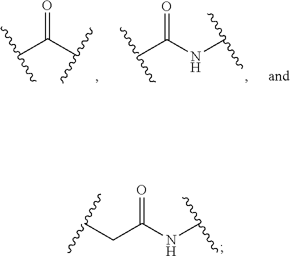

Y is NR¹⁵, CR¹⁶R¹⁷, or oxygen;
R¹⁵ is hydrogen, —C(O)R⁹, C₁₋₆alkyl, or C₃₋₆cycloalkyl;
R¹⁶ and R¹⁷ are independently selected from hydrogen, —C(O)R⁹, C₁₋₆alkyl, and C₃₋₆cycloalkyl; and
R¹ and R² are as defined herein.

In one embodiment, the compound of Formula (I) is a compound of Formula (Ia):

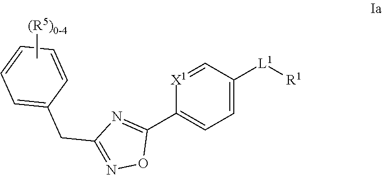

or a pharmaceutically acceptable salt, diastereomer, or stereoisomer thereof; wherein R¹, R⁵, X¹, and L¹ are as defined herein.

In one embodiment, the compound of Formula (I) is a compound of Formula (Ib):

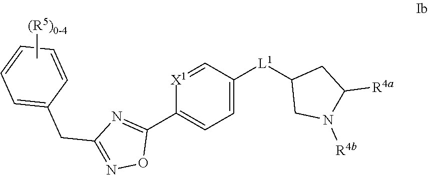

or a pharmaceutically acceptable salt, diastereomer, or stereoisomer thereof; wherein R^{4a}, R^{4b}, R⁵, X¹, and L¹ are as defined herein.

In certain embodiments, the compound of Formula (I) is a compound of Formula (Ic) or Formula (Id):

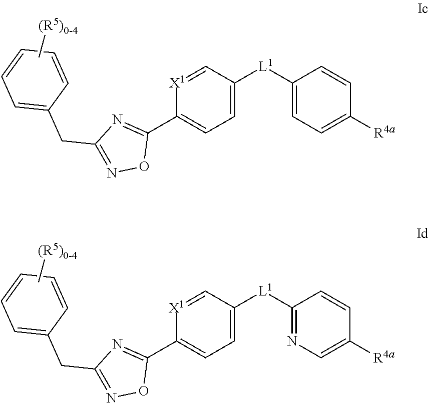

or a pharmaceutically acceptable salt, diastereomer, or stereoisomer thereof; wherein R^{4a}, R⁵, X¹, and L¹ are as defined herein.

In one embodiment, the compound of Formula (II) is a compound of Formula (IIa):

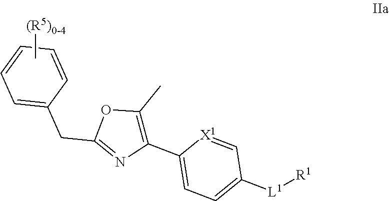

or a pharmaceutically acceptable salt, diastereomer, or stereoisomer thereof; wherein R¹, R⁵, X¹, and L¹ are as defined herein.

In one embodiment, the compound of Formula (II) is a compound of Formula (IIb):

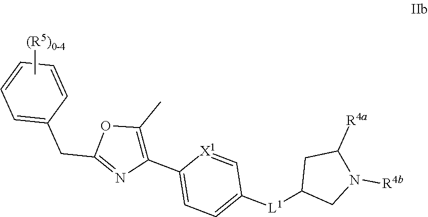

or a pharmaceutically acceptable salt, diastereomer, or stereoisomer thereof; wherein R^{4a}, R^{4b}, R⁵, X¹, and L¹ are as defined herein.

In certain embodiments, the compound of Formula (II) is a compound of Formula (IIc) or (IId):

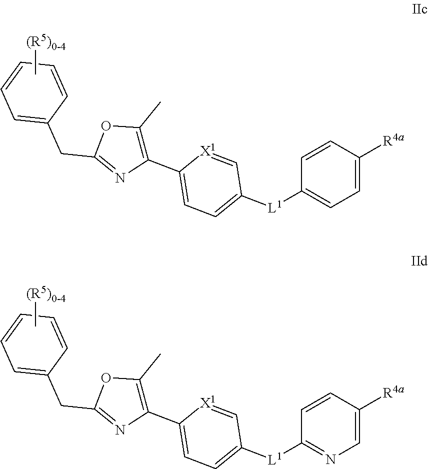

or a pharmaceutically acceptable salt, diastereomer, or stereoisomer thereof; wherein R^{4a}, R⁵, X¹, and L¹ are as defined herein.

In certain aspects, the compounds are useful in methods of treatment and prevention of diseases wherein the inhibition of USP28 is therapeutically beneficial, methods of detection of diseases and conditions wherein the inhibition of USP28 is therapeutically beneficial, and methods of diagnosis of diseases and conditions wherein the inhibition of USP28 is therapeutically beneficial.

In another aspect, provided are compositions comprising a compound of Formula (I), (Ia), (Tb), (Ic), (Id), (II), (IIa), (IIb), (IIc), (IId), or (III). In some embodiments, the compositions are pharmaceutical compositions. Any suitable pharmaceutical composition may be used. In a further aspect, provided herein is a kit comprising a compound of Formula (I), (Ia), (Tb), (Ic), (Id), (II), (IIa), (IIb), (IIc), (IId), or (III), or embodiments thereof, or a pharmaceutical composition thereof.

In another aspect, provided herein are methods of using the compounds or the compositions described herein. In some embodiments, the methods are for treatment. In some embodiments, the methods are diagnostic methods. In some embodiments, the methods are analytical methods. In some embodiments, the compounds, or compositions described herein are used to treat a disease or condition wherein the inhibition of USP28 is therapeutically beneficial. In some aspects, the disease is cancer, including but not limited to, non-small cell lung cancer, breast cancer, intestinal cancer, and bladder cancer. In some aspects, the disease is selected from an inflammatory disease, an autoimmune disease, and an infectious disease.

Also provided herein is the use of compounds described herein, and compositions thereof, for the treatment of a disease or condition wherein the inhibition of USP28 is therapeutically beneficial. Also provided herein is the use of compounds described herein, and compositions thereof, for the treatment of cancer, including, but not limited to, non-small cell lung cancer, breast cancer, intestinal cancer, and bladder cancer.

### DESCRIPTION OF EXEMPLARY EMBODIMENTS

Described herein are USP28 inhibitors useful for treating diseases or conditions wherein the inhibition of USP28 is therapeutically beneficial.

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity, and/or ready reference. The techniques and procedures described or referenced herein are generally well understood, and are commonly employed using conventional methodologies by those skilled in the art. As appropriate, procedures involving the use of commercially available kits, and reagents are generally carried out in accordance with manufacturer-defined protocols, and conditions unless otherwise noted.

As used herein, the singular forms “a,” “an,” and “the” include the plural referents unless the context clearly indicates otherwise.

The term “about” indicates and encompasses an indicated value, and a range above and below that value. In certain embodiments, the term “about” indicates the designated value±10%, +5%, or ±1%. In certain embodiments, the term “about” indicates the designated value±one standard deviation of that value. In certain embodiments, for example, logarithmic scales (e.g., pH), the term “about” indicates the designated value±0.3, ±0.2, or ±0.1.

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise.

“Alkoxy” and “alkoxyl” refer to the group —OR″ where R″ is alkyl or cycloalkyl. Alkoxy groups include, in certain embodiments, methoxy, ethoxy, n-propoxy, isopropoxy, n-butoxy, tert-butoxy, sec-butoxy, n-pentoxy, n-hexoxy, 1,2-dimethylbutoxy, and the like.

The term “alkyl” as used herein, unless otherwise specified, refers to a saturated straight, or branched hydrocarbon. In certain embodiments, the alkyl group is a primary, secondary, or tertiary hydrocarbon. In certain embodiments, the alkyl group includes one to ten carbon atoms (i.e., C₁ to C₁₀ alkyl). In certain embodiments, the alkyl is a lower alkyl, for example, C₁₋₆ alkyl, and the like. In certain embodiments, the alkyl group is selected from the group consisting of methyl, ethyl, propyl, isopropyl, butyl, isobutyl, secbutyl, t-butyl, pentyl, isopentyl, neopentyl, hexyl, isohexyl, 3-methylpentyl, 2,2-dimethylbutyl, and 2,3-dimethylbutyl. In certain embodiments, “substituted alkyl” refers to an alkyl substituted with one, two, or three groups independently selected from a halogen (e.g., fluoro (F), chloro (Cl), bromo (Br), or iodo (I)), alkyl, haloalkyl, hydroxyl, amino, alkylamino, and alkoxy. In some embodiments, alkyl is unsubstituted.

The term “alkylene” as used herein, unless otherwise specified, refers to a divalent alkyl group, as defined herein. “Substituted alkylene” refers to an alkylene group substituted as described herein for alkyl. In some embodiments, alkylene is unsubstituted.

“Alkenyl” refers to an olefinically unsaturated hydrocarbon group, in certain embodiments, having up to about eleven carbon atoms, or from two to six carbon atoms (e.g., “lower alkenyl”), which can be straight-chained or branched, and having at least one, or from one to two sites of olefinic unsaturation. “Substituted alkenyl” refers to an alkenyl group substituted as described herein for alkyl.

“Alkenylene” refers to a divalent alkenyl as defined herein. Lower alkenylene is, for example, C₂-C₆-alkenylene.

“Alkynyl” refers to acetylenically unsaturated hydrocarbon groups, in certain embodiments, having up to about eleven carbon atoms, or from two to six carbon atoms (e.g., “lower alkynyl”), which can be straight-chained or branched, and having at least one, or from one to two sites of acetylenic unsaturation. Non-limiting examples of alkynyl groups include acetylene (—C≡CH), propargyl (—CH₂C≡CH), and the like. “Substituted alkynyl” refers to an alkynyl group substituted as described herein for alkyl.

“Alkynylene” refers to a divalent alkynyl as defined herein. Lower alkynylene is, for example, C₂-C₆-alkynylene.

“Amino” refers to —NH₂.

The term “alkylamino,” as used herein, and unless otherwise specified, refers to the group —NHR″ where R″ is, for example, C₁₋₁₀alkyl, as defined herein. In certain embodiments, alkylamino is C₁₋₆alkylamino.

The term “dialkylamino,” as used herein, and unless otherwise specified, refers to the group —NR″R″ where, each R″ is independently C₁₋₁₀alkyl, as defined herein. In certain embodiments, dialkylamino is di-C₁₋₆alkylamino.

The term “aminoalkyl” as used herein refers to an alkyl group as described herein substituted with an amino group.

The term “alkylaminoalkyl” as used herein refers to an alkyl group as described herein substituted with an alkylamino group as described herein.

The term “dialkylaminoalkyl” as used herein refers to an alkyl group as described herein substituted with an dialkylamino group as described herein.

The term “aryl” as used herein, and unless otherwise specified, refers to phenyl, biphenyl, or naphthyl. The term includes both substituted and unsubstituted moieties. An aryl group can be substituted with any described moiety including, but not limited to, one or more moieties (e.g., in some embodiments one, two, or three moieties) selected from the group consisting of halogen (e.g., fluoro (F), chloro (Cl), bromo (Br), or iodo (I)), alkyl, haloalkyl, hydroxyl, amino, alkylamino, arylamino, alkoxy, aryloxy, nitro, cyano, sulfonic acid, sulfate, phosphonic acid, phosphate, and phosphonate, wherein each moiety is independently either unprotected, or protected as necessary, as would be appreciated by those skilled in the art (e.g., Greene, et al., Protective Groups in Organic Synthesis, John Wiley and Sons, Second Edition, 1991); and wherein the aryl in the arylamino and aryloxy substituents are not further substituted.

The term “arylalkyl” as used herein refers to an “alkyl” group as described herein substituted with an “aryl” group as described herein.

The term “arylamino” as used herein, and unless otherwise specified, refers to an —NR′R″ group where R′ is hydrogen, or C₁-C₆-alkyl; and R″ is aryl, as defined herein.

The term “aryloxy” as used herein, and unless otherwise specified, refers to an —OR group where R is aryl, as defined herein.

The term “aryloxyalkyl,” as used herein, and unless otherwise specified, refers to an alkyl group as described herein substituted with an —OR group where R is aryl, as defined herein.

“Carboxyl” or “carboxy” refers to —C(O)OH or —COOH.

The term “cycloalkyl” as used herein, unless otherwise specified, refers to a saturated cyclic hydrocarbon. In certain embodiments, the cycloalkyl group may be a saturated, and/or bridged, and/or non-bridged, and/or a fused bicyclic group. In certain embodiments, the cycloalkyl group includes three to ten carbon atoms (i.e., C₃ to C₁₀ cycloalkyl). In some embodiments, the cycloalkyl has from three to fifteen carbons (C₃₋₁₅), from three to ten carbons (C₃₋₁₀), from three to seven carbons (C₃₋₇), or from three to six carbons (C₃-C₆) (i.e., “lower cycloalkyl”). In certain embodiments, the cycloalkyl group is cyclopropyl, cyclobutyl, cyclopentyl, cyclohexyl, cyclohexylmethyl, cycloheptyl, bicyclo[2.1.1]hexyl, bicyclo[2.2.1]heptyl, decalinyl, or adamantyl.

The term “ester,” as used herein, refers to —C(O)OR, or —COOR, where R is alkyl, as defined herein.

The term “haloalkyl” refers to an alkyl group, as defined herein, substituted with one, or more halogen atoms (e.g., in some embodiments one, two, three, four, or five) which are independently selected.

The term “halogen” or “halo” as used herein refers to chloro, bromo, iodo, or fluoro.

The term “heteroalkyl” refers to an alkyl, as defined herein, in which one or more carbon atoms are replaced by heteroatoms. As used herein, “heteroalkenyl” refers to an alkenyl, as defined herein, in which one, or more carbon atoms are replaced by heteroatoms. As used herein, “heteroalkynyl” refers to an alkynyl, as defined herein, in which one, or more carbon atoms are replaced by heteroatoms. Suitable heteroatoms include, but are not limited to, nitrogen (N), oxygen (O), and sulfur (S) atoms. Heteroalkyl, heteroalkenyl, and heteroalkynyl are optionally substituted. Examples of heteroalkyl moieties include, but are not limited to, aminoalkyl, sulfonylalkyl, and sulfinylalkyl. Examples of heteroalkyl moieties also include, but are not limited to, methylamino, methylsulfonyl, and methylsulfinyl. “Substituted heteroalkyl” refers to heteroalkyl substituted with one, two, or three groups independently selected from halogen (e.g., fluoro (F), chloro (Cl), bromo (Br), or iodo (I)), alkyl, haloalkyl, hydroxyl, amino, alkylamino, and alkoxy. In some embodiments, a heteroalkyl group may comprise one, two, three, or four heteroatoms. Those of skill in the art will recognize that a 4-membered heteroalkyl may generally comprise one, or two heteroatoms, a 5- or 6-membered heteroalkyl may generally comprise one, two, or three heteroatoms, and a 7- to 10-membered heteroalkyl may generally comprise one, two, three, or four heteroatoms.

The term “heterocyclic” or “heterocycle” refers to a monovalent, monocyclic, or multicyclic non-aromatic ring system, wherein one, or more of the ring atoms are heteroatoms independently selected from oxygen (O), sulfur (S), and nitrogen (N) (e.g., where the nitrogen, or sulfur atoms may be optionally oxidized, and the nitrogen atoms may be optionally quaternized) and the remaining ring atoms of the non-aromatic ring are carbon atoms. In certain embodiments, heterocycle is a monovalent, monocyclic, or multicyclic fully-saturated ring system. In certain embodiments, the heterocyclic group has from three to twenty, from three to fifteen, from three to ten, from three to eight, from four to seven, from four to eleven, or from five to six ring atoms. The heterocycle may be attached to a core structure at any heteroatom or carbon atom which results in the creation of a stable compound. In certain embodiments, the heterocycle is a monocyclic, bicyclic (biheterocyclic), tricyclic (triheterocyclic), or tetracyclic (tetraheterocyclic) ring system, which may include a fused or bridged ring system and in which the nitrogen or sulfur atoms may be optionally oxidized, and/or the nitrogen atoms may be optionally quaternized. In some embodiments, heterocyclic radicals include, but are not limited to, 2,5-diazabicyclo[2.2.2]octanyl, 8-azabicyclo[3.2.1]octanyl, decahydroisoquinolinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, the heterocycle may also be optionally substituted as described herein. In certain embodiments, the heterocycle is substituted with one, two, or three groups independently selected from halogen (e.g., fluoro (F), chloro (Cl), bromo (Br), or iodo (I)), alkyl, haloalkyl, hydroxyl, amino, alkylamino, and alkoxy. In some embodiments, a heterocycle group may comprise one, two, three, or four heteroatoms. Those of skill in the art will recognize that a 4-membered heterocyclic group may generally comprise one or two heteroatoms, a 5 or 6-membered heterocyclic group may generally comprise one, two, or three heteroatoms, and a 7- to 10-membered heterocyclic group may generally comprise one, two, three, or four heteroatoms.

“Biheterocycle” refers to a heterocylic ring system that forms two rings, wherein the rings have at least one atom in common. The biheterocycle group can be fused, bridged, or spirocyclic.

“Heterocycloalkyl” refers to an alkyl group, as used herein, substituted with one or two heterocyclic groups as used herein.

The term “heteroaryl” refers to a monovalent monocyclic aromatic group, and/or multicyclic aromatic group, wherein at least one aromatic ring contains one, or more heteroatoms independently selected from oxygen, sulfur, and nitrogen in the ring. Each ring of a heteroaryl group can contain one, or two oxygen atoms, one, or two sulfur atoms, and/or one to four nitrogen atoms, provided that the total number of heteroatoms in each ring is four, or less and each ring contains at least one carbon atom. In certain embodiments, the heteroaryl has from five to twenty, from five to fifteen, or from five to ten ring atoms. A heteroaryl may be attached to the rest of the molecule via a nitrogen or a carbon atom. In some embodiments, monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, triazolyl, thiadiazolyl, thiazolyl, thienyl, tetrazolyl, and triazinyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may also be optionally substituted as described herein. “Substituted heteroaryl” is a heteroaryl substituted as defined for aryl.

The term “heteroarylalkyl” as used herein refers to an “alkyl” group as described herein substituted with an “heteroaryl” group as described herein.

The term “hydroxyl” refers to —OH.

The term “hydroxyalkyl” as used herein refers to an alkyl group as described herein substituted with a hydroxyl group.

The term “cyano” refers to

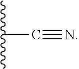 [SMILES: *C#N]

The term “nitro” refers to —NO₂.

The term “oxo” as used herein refers to a keto group (C═O). An oxo group that is a substituent of a nonaromatic carbon results in a conversion of a —CH₂— to a —C═O. An oxo group that is a substituent of an aromatic carbon results in a conversion of —CH— to —C═O. When a substituent is oxo, then two hydrogens of the atom are replaced. When an oxo group substitutes aromatic moieties, the corresponding partially unsaturated ring replaces the aromatic ring. For example, a pyridyl group substituted by an oxo group is a pyridine. A person of skill in the art will appreciate that in some embodiments, such a group (e.g., pyridine), can exist in its tautomeric form (e.g., hydroxypyridine).

The term “protecting group,” as used herein, and unless otherwise specified, refers to a group that is added to an oxygen, nitrogen, or phosphorus atom to prevent its further reaction, or for other purposes. A wide variety of oxygen, and nitrogen protecting groups are known to those skilled in the art of organic synthesis. (See, e.g., Greene, et al., Protective Groups in Organic Synthesis, John Wiley and Sons, Fourth Edition, 2006, which is incorporated herein by reference).

“Pharmaceutically acceptable salt” refers to any salt of a compound provided herein which retains its biological properties, and which is not toxic or otherwise undesirable for pharmaceutical use. Such salts may be derived from a variety of organic, and inorganic counter-ions well known in the art. Such salts include, but are not limited to (1) acid addition salts formed with organic, or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, glutamic, hydroxynaphthoic, salicylic, stearic, cyclohexylsulfamic, quinic, muconic acid, and the like; or (2) salts formed when an acidic proton present in the parent compound either (a) is replaced by a metal ion, for example, an alkali metal ion, an alkaline earth ion, or an aluminum ion, or alkali metal, or alkaline earth metal hydroxides, such as sodium, potassium, calcium, magnesium, aluminum, lithium, zinc, and barium hydroxide, or ammonia; or (b) coordinates with an organic base, such as aliphatic, alicyclic, or aromatic organic amines, including, without limitation, ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, procaine, N-benzylphenethylamine, N-methylglucamine piperazine, tris(hydroxymethyl)-aminomethane, tetramethylammonium hydroxide, and the like.

Pharmaceutically acceptable salts further include, by way of example and without limitation, sodium, potassium, calcium, magnesium, ammonium, and tetraalkylammonium salts, and the like, and when the compound contains a basic functionality, salts of non-toxic organic, or inorganic acids, such as hydrohalides, for example, hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, glutamate, hydroxynaphthoate, salicylate, stearate, cyclohexylsulfamate, quinate, muconate, and the like.

The term “substantially free of” or “substantially in the absence of” with respect to a composition refers to a composition that includes at least 85%, or 90% by weight, in certain embodiments 95%, 98%, 99%, or 100% by weight; or in certain embodiments, 95%, 98%, 99%, or 100% of the designated enantiomer or diastereomer of a compound. In certain embodiments, in the methods and compounds provided herein, the compounds are substantially free of one of two enantiomers. In certain embodiments, in the methods and compounds provided herein, the compounds are substantially free of one of two diastereomers. In certain embodiments, in the methods, and compounds provided herein, the compounds are substantially free of enantiomers (i.e., a racemic, or 50:50 mixture of compounds).

Similarly, the term “isolated” with respect to a composition refers to a composition that includes at least 85%, 90%, 95%, 98%, or 99% to 100% by weight, of the compound, the remainder comprising other chemical species, enantiomers or diastereomers.

“Solvate” refers to a compound provided herein, or a salt thereof, that further includes a stoichiometric, or non-stoichiometric amount of solvent bound by non-covalent intermolecular forces. Where the solvent is water, the solvate is a hydrate.

“Isotopic composition” refers to the amount of each isotope present for a given atom, and “natural isotopic composition” refers to the naturally occurring isotopic composition, or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as hydrogen (H), the position is understood to have hydrogen at its natural isotopic composition.

“Isotopic enrichment” refers to the percentage of incorporation of an amount of a specific isotope at a given atom in a molecule in the place of that atom's natural isotopic abundance. For example, deuterium (D) enrichment of 10% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

“Isotopically enriched” refers to an atom having an isotopic composition other than the natural isotopic composition of that atom. “Isotopically enriched” may also refer to a compound containing at least one atom having an isotopic composition other than the natural isotopic composition of that atom.

As used herein, “alkyl,” “alkylene,” “alkylamino,” “dialkylamino,” “cycloalkyl,” “aryl,” “arylene,” “alkoxy,” “amino,” “carboxyl,” “heterocycloalkyl,” “heteroaryl,” “carboxyl,” and “amino acid” groups optionally comprise deuterium (D) at one, or more positions where hydrogen (H) atoms are present, and wherein the deuterium composition of the atom or atoms is other than the natural isotopic composition.

Also as used herein, “alkyl,” “alkylene,” “alkylamino,” “dialkylamino,” “cycloalkyl,” “aryl,” “arylene,” “alkoxy,” “amino,” “carboxyl,” “heterocycloalkyl,” “heteroaryl,” “carboxyl,” and “amino acid” groups optionally comprise carbon-13 (¹³C) at an amount other than the natural isotopic composition.

As used herein, term “EC₅₀” refers to a dosage, concentration, or amount of a particular test compound that elicits a dose-dependent response at 50% of maximal expression of a particular response that is induced, provoked, or potentiated by the particular test compound.

As used herein, and unless otherwise specified, the term “IC₅₀” refers to an amount, concentration, or dosage of a particular test compound that achieves a 50% inhibition of a maximal response in an assay that measures such response.

As used herein, the terms “subject”, “patient,” and “host” are used interchangeably. The terms “subject”, and “subjects” refer to an animal, such as a mammal including a non-primate (e.g., a cow, pig, horse, cat, dog, rat, and mouse), and a primate (e.g., a monkey, such as a cynomolgous monkey, a chimpanzee, and a human), and in certain embodiments, a human. In certain embodiments, the subject is a farm animal (e.g., a horse, a cow, a pig, etc.), or a pet (e.g., a dog or a cat). In certain embodiments, the subject is a human.

As used herein, the terms “therapeutic agent”, and “therapeutic agents” refer to any agent(s) which can be used in the treatment, or prevention of a disorder, or one or more symptoms thereof. In certain embodiments, the term “therapeutic agent” includes a compound provided herein. In certain embodiments, a therapeutic agent is an agent which is known to be useful for, or has been, or is currently being used for the treatment, or prevention of a disorder, or one, or more symptoms thereof.

“Therapeutically effective amount” refers to an amount of a compound, or composition that, when administered to a subject for treating a condition, is sufficient to effect such treatment for the condition. A “therapeutically effective amount” can vary depending on, inter alia, the compound, the disease or disorder, and its severity, and the age, weight, etc., of the subject to be treated.

“Treating” or “treatment” of any disease, or disorder refers, in certain embodiments, to ameliorating a disease, or disorder that exists in a subject. In another embodiment, “treating”, or “treatment” includes ameliorating at least one physical parameter, which may be indiscernible by the subject. In yet another embodiment, “treating”, or “treatment” includes modulating the disease, or disorder, either physically (e.g., stabilization of a discernible symptom), or physiologically (e.g., stabilization of a physical parameter), or both. In yet another embodiment, “treating”, or “treatment” includes delaying, or preventing the onset of the disease, or disorder, or delaying, or preventing recurrence of the disease, or disorder. In yet another embodiment, “treating”, or “treatment” includes the reduction or elimination of either the disease, or disorder, or retarding the progression of the disease, or disorder, or of one, or more symptoms of the disease, or disorder, or reducing the severity of the disease, or disorder, or of one, or more symptoms of the disease, or disorder.

As used herein, the terms “prophylactic agent”, and “prophylactic agents” as used refer to any agent(s) which can be used in the prevention of a disorder, or one or more symptoms thereof. In certain embodiments, the term “prophylactic agent” includes a compound provided herein. In certain other embodiments, the term “prophylactic agent” does not refer a compound provided herein. For example, a prophylactic agent is an agent which is known to be useful for, or has been or is currently being used to prevent, or impede the onset, development, progression, and/or severity of a disorder.

As used herein, the phrase “prophylactically effective amount” refers to the amount of a therapy (e.g., prophylactic agent) which is sufficient to result in the prevention, or reduction of the development, recurrence, or onset of one or more symptoms associated with a disorder, or to enhance or improve the prophylactic effect(s) of another therapy (e.g., another prophylactic agent).

### Compounds of Formula (I), (Ia), (Ib), (Ic), (Id), (II), (IIa), (IIb), (II), (IId), and (III)

Provided herein are USP28 inhibitors useful for diseases wherein the inhibition of USP28 is therapeutically beneficial. The compounds can be prepared as described herein and used for therapy or diagnosis. In certain embodiments, the therapy is the treatment of cancer, including, but not limited to, non-small cell lung cancer, breast cancer, intestinal cancer, and bladder cancer. In certain embodiments, the therapy is the treatment of an autoimmune disease, including, but not limited to, intestine inflammatory disease, ulcerative colitis, Crohn's disease, and rheumatoid arthritis. In certain embodiments, the therapy is the treatment of inflammation. In certain embodiments, the therapy is the treatment of an infectious disease, including but not limited to, a viral infection and a bacterial infection. In certain embodiments, the therapy is the treatment of neurodegenerative diseases.

The embodiments described herein include the recited compounds as well as pharmaceutically acceptable salts, hydrates, solvates, stereoisomers, diastereomers, tautomers, and/or mixtures thereof.

In certain embodiments, L¹ is

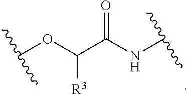

In certain embodiments, L¹ is

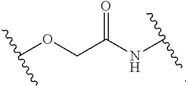 [SMILES: *NC(=O)CO*]

In certain embodiments, L¹ is

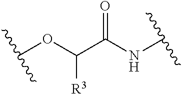

and R³ is C₁₋₆alkyl. In certain embodiments, L¹ is

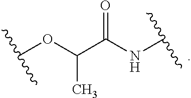 [SMILES: *NC(=O)C(C)O*]

In certain embodiments, L¹ is

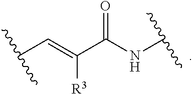

In certain embodiments, L¹ is

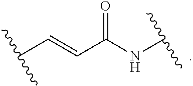 [SMILES: */C=C/C(=O)N*]

In certain embodiments, L¹ is

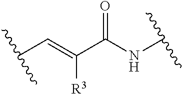

and R³ is C₁₋₆alkyl. In certain embodiments, L¹ is

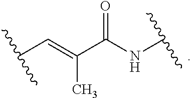 [SMILES: */C=C(\C)C(=O)N*]

In certain embodiments, L¹ is

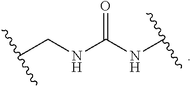 [SMILES: *CNC(=O)N*]

In certain embodiments, L¹ is

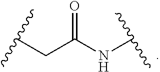 [SMILES: *CC(=O)N*]

In certain embodiments, L¹ is

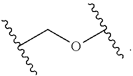 [SMILES: *CO*]

In certain embodiments, L¹ is

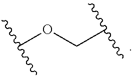 [SMILES: *CO*]

In certain embodiments, R¹ is aryl substituted with one R^{4a} group. In certain embodiments, R¹ is aryl substituted with two R^{4a} groups. In certain embodiments, R¹ is aryl substituted with —C(O)NR⁶R⁷. In certain embodiments, R¹ is aryl substituted with —C(O)N(C₁₋₆ alkyl)₂. In certain embodiments, R¹ is aryl substituted with —C(O)N(Me)₂. In certain embodiments, R¹ is aryl substituted with C₁₋₆alkyl. In certain embodiments, R¹ is aryl substituted with methyl. In certain embodiments, R¹ is aryl substituted with —CH₂NR⁶R⁷. In certain embodiments, R¹ is aryl substituted with —CH₂N(C₁₋₆alkyl)₂. In certain embodiments, R¹ is aryl substituted with —CH₂N(Me)₂. In certain embodiments, R¹ is aryl substituted with —C(O)NR⁶R⁷ wherein R⁶ is hydrogen and R⁷ is heterocycloC₁₋₆alkyl. In certain embodiments, R¹ is aryl substituted with —C(O)NR⁶R⁷ wherein R⁶ and R⁷ are joined together to form a heterocycle optionally substituted with R¹⁰. In certain embodiments, R¹ is aryl substituted with —C(O)NR⁶R⁷ wherein R⁶ and R⁷ are joined together to form a biheterocycle optionally substituted with R¹⁰. In certain embodiments, R¹ is aryl substituted with —NR⁶R⁷. In certain embodiments, R¹ is aryl substituted with —N(C₁₋₆alkyl)₂. In certain embodiments, R¹ is aryl substituted with —N(Me)₂. In certain embodiments, R¹ is aryl substituted with —NH₂. In certain embodiments, R¹ is aryl substituted with —NHMe. In certain embodiments, R¹ is aryl substituted with —(CH₂)ₐ—O—(CH₂)_{b}R⁸ wherein R⁸ is hydroxy. In certain embodiments, R¹ is aryl substituted with —(CH₂)ₐ—O—(CH₂)_{b}R⁸ wherein a is 1 and b is 2. In certain embodiments, R¹ is aryl substituted with —(CH₂)ₐ—O—(CH₂)_{b}R⁸ wherein a is 1; b is 2; and R⁸ is hydroxy. In certain embodiments, R¹ is aryl substituted with —(CH₂)ₐ—O—(CH₂)_{b}R⁸ and a second R^{4a}. In one embodiment, the R^{4a} group is —C(O)NR⁶R⁷.

In certain embodiments, R¹ is cycloalkyl substituted with one R^{4a} group. In certain embodiments, R¹ is cycloalkyl substituted with two R^{4a} groups. In certain embodiments, R¹ is aryl substituted with —C(O)NR⁶R⁷. In certain embodiments, R¹ is cycloalkyl substituted with —C(O)N(C₁₋₆alkyl)₂. In certain embodiments, R¹ is cycloalkyl substituted with —C(O)N(Me)₂. In certain embodiments, R¹ is cycloalkyl substituted with C₁₋₆alkyl. In certain embodiments, R¹ is cycloalkyl substituted with methyl. In certain embodiments, R¹ is cycloalkyl substituted with —CH₂NR⁶R⁷. In certain embodiments, R¹ is cycloalkyl substituted with —CH₂N(Me)₂. In certain embodiments, R¹ is cycloalkyl substituted with —NR⁶R⁷. In certain embodiments, R¹ is cycloalkyl substituted with —N(Me)₂. In certain embodiments, R¹ is cycloalkyl substituted with —NH₂. In certain embodiments, R¹ is cycloalkyl substituted with —NHMe.

In certain embodiments, R¹ is heteroaryl substituted with one R^{4a} group. In certain embodiments, R¹ is heteroaryl substituted with two R^{4a} groups. In certain embodiments, R¹ is heteroaryl substituted with —C(O)NR⁶R⁷. In certain embodiments, R¹ is heteroaryl substituted with —C(O)N(C₁₋₆alkyl)₂. In certain embodiments, R¹ is heteroaryl substituted with —C(O)N(Me)₂. In certain embodiments, R¹ is heteroaryl substituted with C₁₋₆alkyl. In certain embodiments, R¹ is heteroaryl substituted with methyl. In certain embodiments, R¹ is heteroaryl substituted with —CH₂NR⁶R⁷. In certain embodiments, R¹ is heteroaryl substituted with —CH₂N(C₁₋₆alkyl)₂. In certain embodiments, R¹ is heteroaryl substituted with —CH₂N(Me)₂. In certain embodiments, R¹ is heteroaryl substituted with —C(O)NR⁶R⁷ wherein R⁶ is hydrogen and R⁷ is heterocycloC₁₋₆alkyl. In certain embodiments, R¹ is heteroaryl substituted with —C(O)NR⁶R⁷ wherein R⁶ and R⁷ are joined together to form a heterocycle optionally substituted with R¹⁰. In certain embodiments, R¹ is heteroaryl substituted with —C(O)NR⁶R⁷ wherein R⁶ and R⁷ are joined together to form a biheterocycle optionally substituted with R¹⁰. In certain embodiments, R¹ is heteroaryl substituted with —NR⁶R⁷. In certain embodiments, R¹ is heteroaryl substituted with —N(C₁₋₆alkyl)₂. In certain embodiments, R¹ is heteroaryl substituted with —N(Me)₂. In certain embodiments, R¹ is heteroaryl substituted with —NH₂. In certain embodiments, R¹ is heteroaryl substituted with —NHMe.

In certain embodiments, R¹ is heteroaryl or heterocycle substituted with one or two R^{4a} groups wherein the heteroaryl or heterocycle contains one nitrogen. In certain embodiments, R¹ is heteroaryl or heterocycle substituted with one or two R^{4a} groups wherein the heteroaryl or heterocycle contains two nitrogen atoms. In certain embodiments, R¹ is heteroaryl or heterocycle substituted with one or two R^{4a} groups wherein the hetereoaryl or heterocycle contains three nitrogen atoms. In certain embodiments, R¹ is heteroaryl or heterocycle substituted with one or two R^{4a} groups wherein the hetereoaryl or heterocycle contains one nitrogen and one oxygen. In certain embodiments, R¹ is heteroaryl or heterocycle substituted with one or two R^{4a} groups wherein the hetereoaryl or heterocycle contains one nitrogen and one sulfur. In certain embodiments, R¹ is heteroaryl or heterocycle substituted with one or two R^{4a} groups wherein the hetereoaryl or heterocycle contains two nitrogen atoms and one sulfur. In certain embodiments, R¹ is heteroaryl or heterocycle substituted with one or two R^{4a} groups wherein the hetereoaryl or heterocycle contains two nitrogen atoms and one oxygen. In certain embodiments, R¹ is heteroaryl or heterocycle substituted with one or two R^{4a} groups wherein the hetereoaryl or heterocycle contains one oxygen and one sulfur. In certain embodiments, R¹ is heteroaryl or heterocycle substituted with one or two R^{4a} groups wherein the hetereoaryl or heterocycle contains one sulfur. In certain embodiments, R¹ is heteroaryl or heterocycle substituted with one or two R^{4a} groups wherein the hetereoaryl or heterocycle contains one oxygen.

In certain embodiments, R¹ is heterocycloalkyl substituted with one R^{4a} group and R^{4b} is hydrogen. In certain embodiments, R¹ is heterocycloalkyl substituted with two R^{4a} groups and R^{4b} is hydrogen. In certain embodiments, R¹ is heterocycloalkyl substituted with one R^{4a} group and R^{4b} is cyano. In certain embodiments, R¹ is heterocycloalkyl substituted with two R^{4a} groups and R^{4b} is cyano. In certain embodiments, R¹ is heterocycloalkyl substituted with C₁₋₆haloalkyl and R^{4b} is hydrogen. In certain embodiments, R¹ is heterocycloalkyl substituted with C₁₋₆haloalkyl and R^{4b} is cyano. In certain embodiments, R¹ is heterocycloalkyl substituted with C₁₋₆alkyl and R^{4b} is hydrogen. In certain embodiments, R¹ is heterocycloalkyl substituted with C₁₋₆alkyl and R^{4b} is cyano. In certain embodiments, R¹ is heterocycloalkyl substituted with methyl and R^{4b} is cyano.

In certain embodiments, R¹ is selected from:

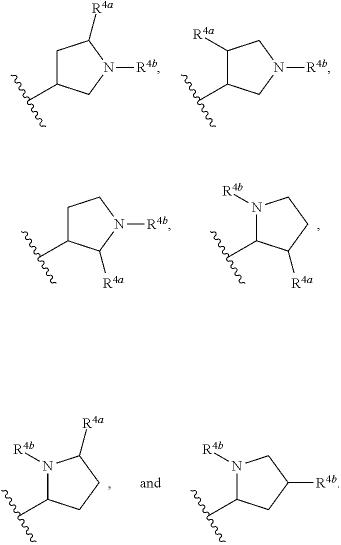

In certain embodiments, R¹ is selected from:

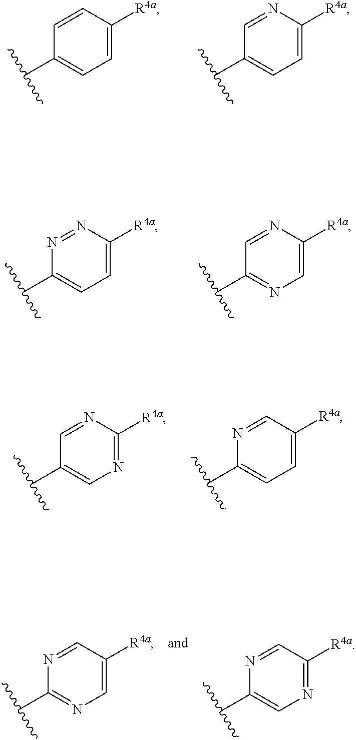

In certain embodiments, R¹ is selected from:

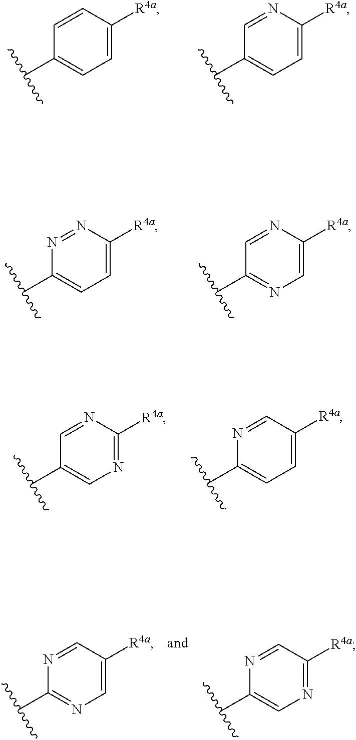

R^{4a} is selected from —C(O)NR⁶R⁷, C₁₋₆alkyl, and —CH₂NR⁶R⁷;
R⁶ and R⁷ are independently selected from hydrogen, C₁₋₆alkyl, and heteroarylC₁₋₆alkyl;
or R⁶ and R⁷ are joined together to form a heterocycle or biheterocycle optionally substituted with R¹⁰; and
R¹⁰ is selected from —COOH, —NH₂, —NHMe, and heteroarylC₁₋₆alkyl.

In certain embodiments, R¹ is selected from:

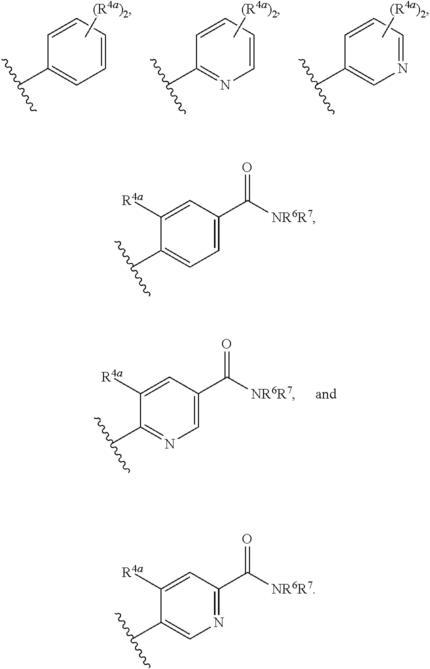

In certain embodiments, R¹ is selected from:

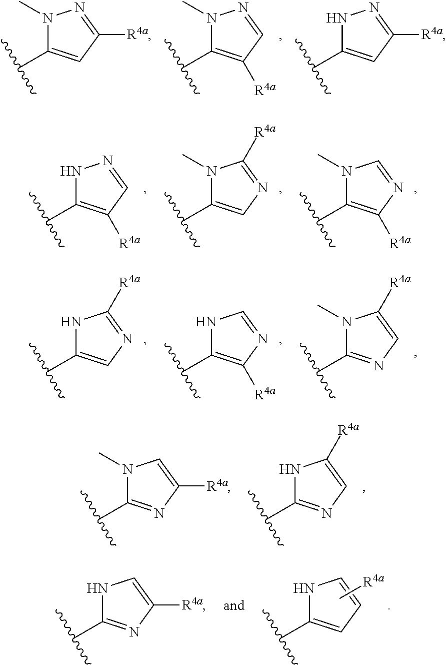

In certain embodiments, R¹ is

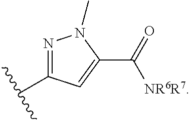

In certain embodiments, R¹ is

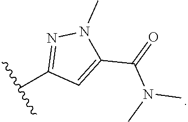 [SMILES: *c1cc(C(=O)N(C)C)n(C)n1]

In certain embodiments, R¹ is

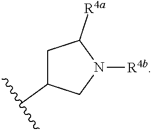

In certain embodiments, R^{4b} is cyano. In certain embodiments, R^{4b} is hydrogen. In certain embodiments, R^{4b} is cyano and R^{4a} is hydrogen. In certain embodiments, R^{4b} is cyano and R^{4a} is C₁₋₆alkyl. In certain, R^{4b} is cyano and R^{4a} is methyl. In certain embodiments, R^{4b} is cyano and R^{4a} is C₁₋₆haloalkyl. In certain embodiments, R^{4b} is cyano and R^{4a} is CH₂F. In certain embodiments, R^{4b} is hydrogen and R^{4a} is methyl.

In certain embodiments, R¹ is

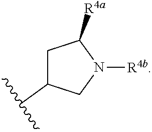

In certain embodiments, R¹ is

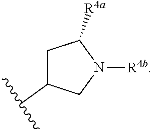

In certain embodiments, R¹ is

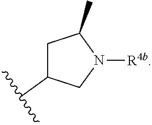

In certain embodiments, R¹ is

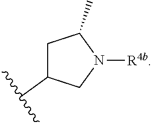

In certain embodiments, R¹ is selected from

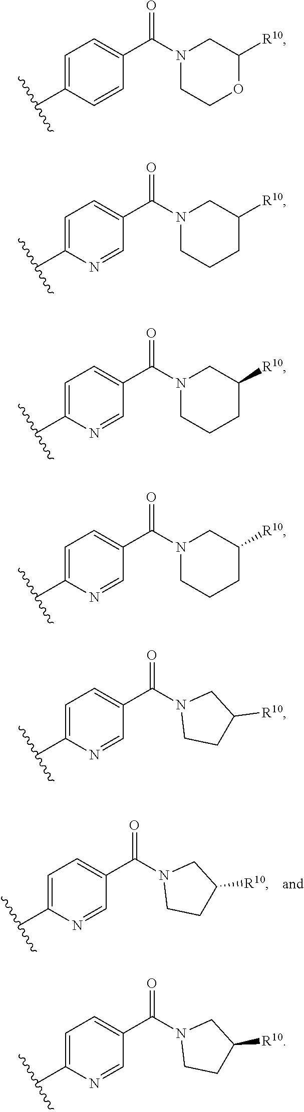

In certain embodiments, R¹ is selected from

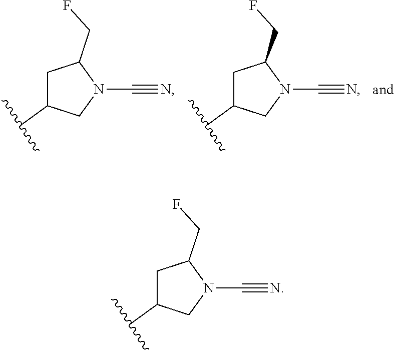 [SMILES: *C1CC(CF)N(C#N)C1 ; *C1C[C@@H](CF)N(C#N)C1 ; *C1CC(CF)N(C#N)C1]

In certain embodiments, R¹ is selected from

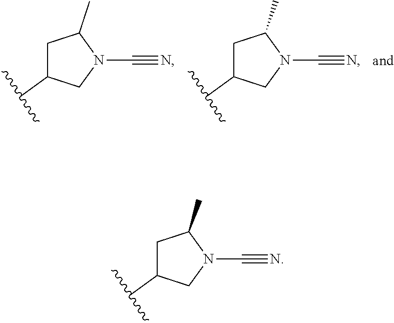

In certain embodiments, R¹ is selected from

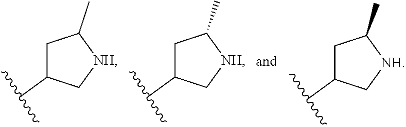

In certain embodiments, R is selected from

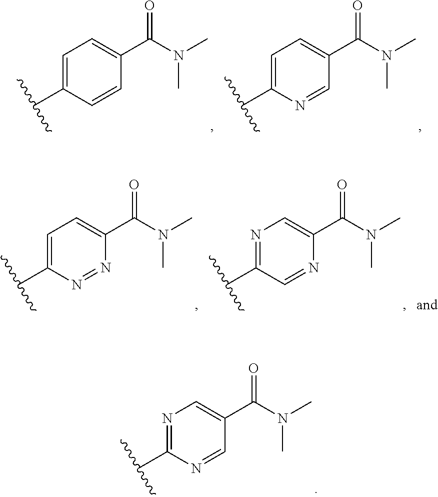 [SMILES: *c1ccc(C(=O)N(C)C)cc1 ; *c1ccc(C(=O)N(C)C)cn1 ; *c1ccc(C(=O)N(C)C)nn1 ; *c1cnc(C(=O)N(C)C)cn1 ; *c1ncc(C(=O)N(C)C)cn1]

In certain embodiments, R¹ is selected from

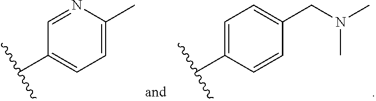

In certain embodiments, R¹ is selected from

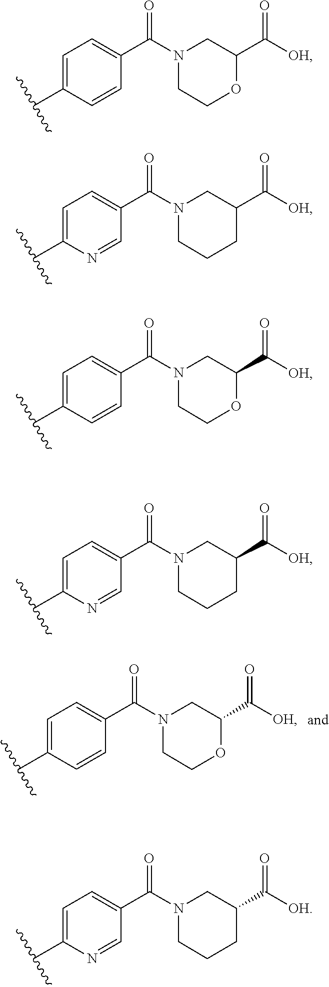 [SMILES: *c1ccc(C(=O)N2CCOC(C(=O)O)C2)cc1 ; *c1ccc(C(=O)N2CCCC(C(=O)O)C2)cn1 ; *c1ccc(C(=O)N2CCO[C@H](C(=O)O)C2)cc1 ; *c1ccc(C(=O)N2CCC[C@H](C(=O)O)C2)cn1 ; *c1ccc(C(=O)N2CCO[C@@H](C(=O)O)C2)cc1 ; *c1ccc(C(=O)N2CCC[C@@H](C(=O)O)C2)cn1]

In certain embodiments, R¹ is selected from

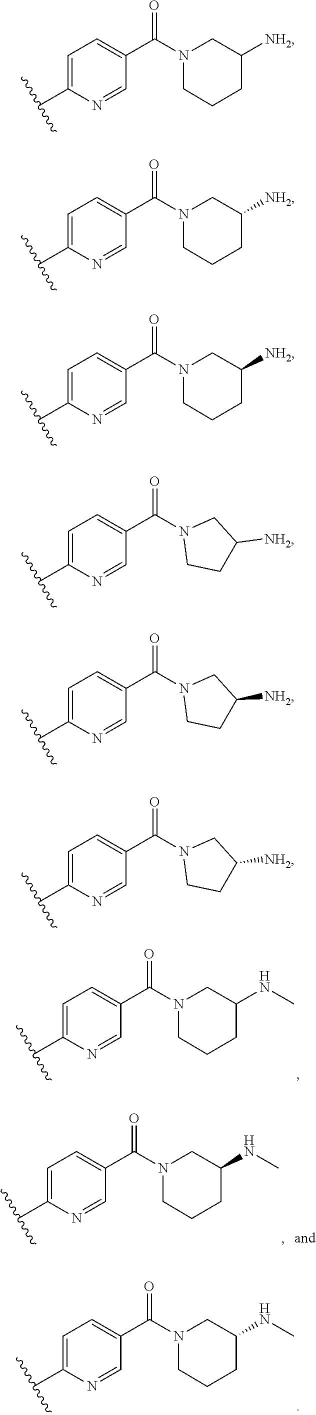

In certain embodiments, R¹ is selected from

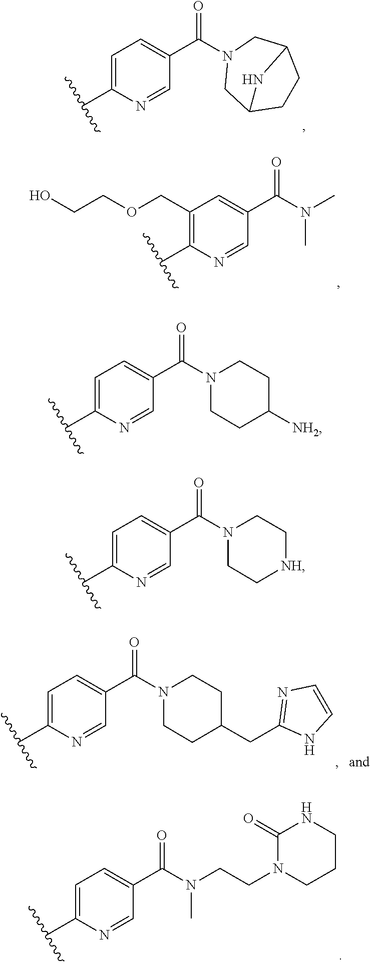 [SMILES: *c1ccc(C(=O)N2CC3CCC(C2)N3)cn1 ; *c1ncc(C(=O)N(C)C)cc1COCCO ; *c1ccc(C(=O)N2CCC(N)CC2)cn1 ; *c1ccc(C(=O)N2CCNCC2)cn1 ; *c1ccc(C(=O)N2CCC(Cc3ncc[nH]3)CC2)cn1 ; *c1ccc(C(=O)N(C)CCN2CCCNC2=O)cn1]

In certain embodiments, R² is heteroaryl or heterocycle substituted with one or two R⁵ groups wherein the heteroaryl or heterocycle contains one nitrogen. In certain embodiments, R² is heteroaryl or heterocycle substituted with one or two R⁵ groups wherein the hetereoaryl or heterocycle contains two nitrogen atoms. In certain embodiments, R² is heteroaryl or heterocycle substituted with one or two R⁵ groups wherein the hetereoaryl or heterocycle contains one nitrogen and one oxygen. In certain embodiments, R² is heteroaryl or heterocycle substituted with one or two R⁵ groups wherein the hetereoaryl or heterocycle contains one nitrogen and one sulfur. In certain embodiments, R² is heteroaryl or heterocycle substituted with one or two R⁵ groups wherein the hetereoaryl or heterocycle contains two nitrogen atoms and one sulfur. In certain embodiments, R² is heteroaryl or heterocycle substituted with one or two R⁵ groups wherein the hetereoaryl or heterocycle contains two nitrogen atoms and one oxygen. In certain embodiments, R² is heteroaryl or heterocycle substituted with one or two R⁵ groups wherein the hetereoaryl or heterocycle contains one oxygen and one sulfur. In certain embodiments, R² is heteroaryl or heterocycle substituted with one or two R⁵ groups wherein the hetereoaryl or heterocycle contains one sulfur. In certain embodiments, R² is heteroaryl or heterocycle substituted with one or two R⁵ groups wherein the hetereoaryl or heterocycle contains one oxygen.

In certain embodiments, R² is aryl substituted with one R⁵ group. In certain embodiments, R² is aryl substituted with two R⁵ groups. In certain embodiments, R² is aryl substituted with halogen. In certain embodiments, R² is aryl substituted with chlorine. In certain embodiments, R² is aryl substituted with C₁₋₆alkyl. In certain embodiments, R² is aryl substituted with methyl. In certain embodiments, R² is aryl substituted with C₁₋₆haloalkyl. In certain embodiments, R² is aryl substituted with CF₃. In certain embodiment, R² is aryl substituted with C₁₋₆alkoxy. In certain embodiments, R² is aryl substituted with methoxy. In certain embodiments, R² is aryl substituted with cyano.

In certain embodiments, R² is heteroaryl substituted with one R⁵ group. In certain embodiments, R² is heteroaryl substituted with two R⁵ groups. In certain embodiments, R² is heteroaryl substituted with halogen. In certain embodiments, R² is heteroaryl substituted with chlorine. In certain embodiments, R² is heteroaryl substituted with C₁₋₆alkyl. In certain embodiments, R² is heteroaryl substituted with methyl. In certain embodiments, R² is heteroaryl substituted with C₁₋₆haloalkyl. In certain embodiments, R² is heteroaryl substituted with CF₃. In certain embodiments, R² is heteroaryl substituted with C₁₋₆alkoxy. In certain embodiments, R² is heteroaryl substituted with methoxy. In certain embodiments, R² is heteroaryl substituted with cyano.

In certain embodiments, R² is

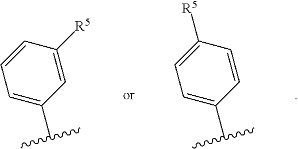

In certain embodiments, R² is selected from

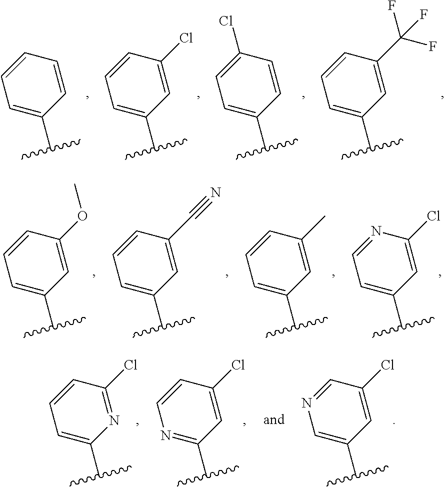

In certain embodiments, R² is

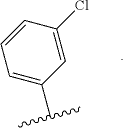 [SMILES: *c1cccc(Cl)c1]

In certain embodiments, R² is

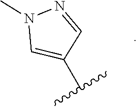 [SMILES: *c1cnn(C)c1]

In certain embodiments, R² is aryl optionally substituted with one R⁵ group and R¹ is heterocycle substituted with one R^{4a} group. In certain embodiments, R² is aryl optionally substituted with one R⁵ group and R¹ is aryl substituted with one R^{4a} group. In certain embodiments, R² is aryl optionally substituted with one R⁵ group and R¹ is heteroaryl substituted with one R^{4a} group. In certain embodiments, R² is heteroaryl optionally substituted with one R⁵ group and R¹ is heteraryl substituted with one R^{4a} group.

In certain embodiments, R² is

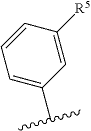

### and R¹ is

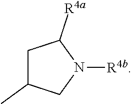

In certain embodiments, R² is

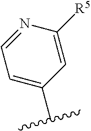

### and R¹ is

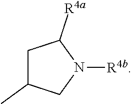

In certain embodiments, R² is

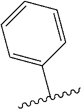 [SMILES: *c1ccccc1]

### and R¹ is

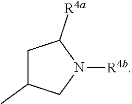

In certain embodiments, R² is

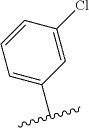 [SMILES: *c1cccc(Cl)c1]

### and R¹ is

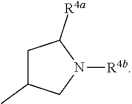

In certain embodiments, R² is

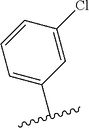 [SMILES: *c1cccc(Cl)c1]

### and R¹ is

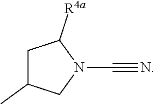

In certain embodiments, R² is

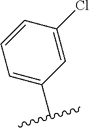 [SMILES: *c1cccc(Cl)c1]

### and R¹ is

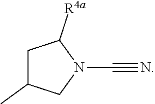

In certain embodiments, R² is

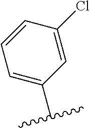 [SMILES: *c1cccc(Cl)c1]

### and R¹ is

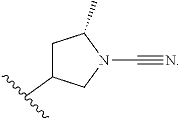 [SMILES: *C1C[C@H](C)N(C#N)C1]

In certain embodiments, R² is

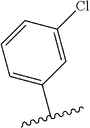 [SMILES: *c1cccc(Cl)c1]

### and R¹ is

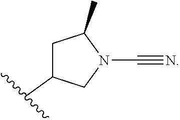 [SMILES: *C1C[C@@H](C)N(C#N)C1]

In certain embodiments, R² is

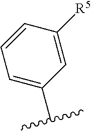

### and R¹ is

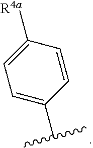

In certain embodiments, R² is

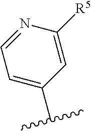

### and R¹ is

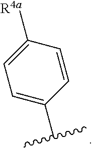

In certain embodiments, R² is

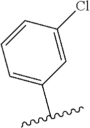 [SMILES: *c1cccc(Cl)c1]

### and R¹ is

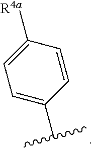

In certain embodiments, R² is

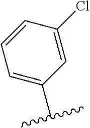 [SMILES: *c1cccc(Cl)c1]

### and R¹ is

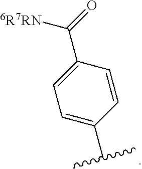

In certain embodiments, R² is

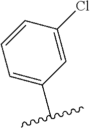 [SMILES: *c1cccc(Cl)c1]

### and R¹ is

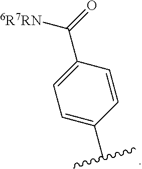

In certain embodiments, R^{4a} is —NR⁶R⁷, —CH₂NR⁶R⁷, or —C(O)NR⁶R⁷ and R⁶ and R⁷ are joined together to form a biheterocycle that contains at least one oxygen, nitrogen or sulfur including the nitrogen to which R⁶ and R⁷ are attached. In certain embodiments, the biheterocycle is a bridged heterocycle. In certain embodiments, the bridged heterocycle contains one nitrogen which is the nitrogen to which R⁶ and R⁷ are attached. In certain embodiments, the bridged heterocycle contains two nitrogen atoms, one of which is the nitrogen to which R⁶ and R⁷ are attached. In certain embodiments, the bridged heterocycle is selected from quinuclidine, adamantane, 8-azabicyclo[3.2.1]octane, and 1,4-diazabicyclo[2.2.2]octane. In certain embodiments, R^{4a} is —NR⁶R⁷, —CH₂NR⁶R⁷, or —C(O)NR⁶R⁷ and R⁶ and R⁷ are joined together to form a heterocycle that contains at least one oxygen, nitrogen or sulfur including the nitrogen to which R⁶ and R⁷ are attached. In certain embodiments, the heterocycle contains one nitrogen and one oxygen. In certain embodiments, the heterocycle contains one nitrogen. In certain embodiments, the heterocycle contains two nitrogen atoms. In certain embodiments, the heterocycle contains three nitrogen atoms. In certain embodiments, R⁶ and R⁷ are joined together to form a morpholine optionally substituted with R¹⁰. In certain embodiments, R⁶ and R⁷ are joined together to form a piperidine optionally substituted with R¹⁰. In certain embodiments, R⁶ and R⁷ are joined together to form a pyrrolidine optionally substituted with R¹⁰. In certain embodiments, R⁶ and R⁷ are joined together to form a piperazine optionally substituted with R¹⁰. In certain embodiments, R¹⁰ is COOH. In certain embodiments, R¹⁰ is amino. In certain embodiments, R¹⁰ is —NHMe. In certain embodiments, R¹⁰ is heterocycloC₁₋₆alkyl. In certain embodiments, the heterocycloC₁₋₆alkyl is selected from a pyrazole, an imidazole, an imidazoline, a pyrazoline, an imidazolidine, a pyrazolidine, a pyrrole, a pyrroline, and a pyrrolidine.

In certain embodiments, R^{4a} is —NR⁶R⁷, —CH₂NR⁶R⁷, or —C(O)NR⁶R⁷ and R⁶ and R⁷ are independently selected from hydrogen and C₁₋₆alkyl. In certain embodiments, R^{4a} is —C(O)NR⁶R⁷ and R⁶ and R⁷ are independently selected from hydrogen and C₁₋₆alkyl. In certain embodiments, R^{4a} is —C(O)NR⁶R⁷ and R⁶ and R⁷ are both C₁₋₆alkyl. In certain embodiments, R^{4a} is —C(O)NR⁶R⁷ and R⁶ and R⁷ are both methyl. In certain embodiments, R^{4a} is —C(O)NR⁶R⁷ and R⁶ is C₁₋₆alkyl and R⁷ is heterocycloC₁₋₆alkyl. In certain embodiments, R^{4a} is —C(O)NR⁶R⁷ and R⁶ is C₁₋₆alkyl and R⁷ is heterocycloC₂alkyl. In certain embodiments, R^{4a} is —C(O)NR⁶R⁷ and R⁶ is methyl and R⁷ is heterocycloC₂alkyl. In certain embodiments, R^{4a} is —C(O)NR⁶R⁷ and R⁶ is C₁₋₆alkyl and R⁷ is heterocycloC₂alkyl substituted with 2 R¹⁰ groups wherein the 2R¹⁰ groups are taken together to form an oxo group.

In certain embodiments, R^{4a} is —(CH₂)ₐ—O—(CH₂)_{b}R⁸ wherein a is 1 and b is 1. In certain embodiments, R^{4a} is —(CH₂)ₐ—O—(CH₂)_{b}R⁸ wherein a is 1 and b is 2. In certain embodiments, R^{4a} is —(CH₂)ₐ—O—(CH₂)_{b}R⁸ wherein a is 1 and b is 3 or 4. In certain embodiments, R^{4a} is —(CH₂)ₐ—O—(CH₂)_{b}R⁸ wherein a is 2 and b is 1. In certain embodiments, R^{4a} is —(CH₂)ₐ—O—(CH₂)_{b}R⁸ wherein a is 2 and b is 2. In certain embodiments, R^{4a} is —(CH₂)ₐ—O—(CH₂)_{b}R⁸ wherein a is 2 and b is 3 or 4. In certain embodiments, R⁸ is OH. In certain embodiments, R⁸ is COOH. In certain embodiments, R⁸ is amino.

In certain embodiments, R⁶ and R⁷ are joined together to form a heterocycle that contains at least one oxygen, nitrogen or sulfur including the nitrogen to which R⁶ and R⁷ are attached. In certain embodiments, R⁶ and R⁷ are joined together to form a heterocycle that contains one nitrogen which is the nitrogen to which R⁶ and R⁷ are attached. In certain embodiments, R⁶ and R⁷ are joined together to form a heterocycle that contains at two nitrogen atoms and one of which is the nitrogen to which R⁶ and R⁷ are attached. In certain embodiments, R⁶ and R⁷ are joined together to form a heterocycle that contains at one nitrogen and one oxygen wherein the nitrogen is the nitrogen to which R⁶ and R⁷ are attached. In certain embodiments, R⁶ and R⁷ are joined together to form a biheterocycle that contains at least one oxygen, nitrogen or sulfur including the nitrogen to which R⁶ and R⁷ are attached. In certain embodiments, R⁶ and R⁷ are joined together to form a biheterocycle that contains one nitrogen which is the nitrogen to which R⁶ and R⁷ are attached. In certain embodiments, R⁶ and R⁷ are joined together to form a biheterocycle that contains at least two nitrogen atoms, one of which is the nitrogen to which R⁶ and R⁷ are attached. In certain embodiments, R⁶ and R⁷ are joined together to form a biheterocycle that contains one nitrogen and one oxygen wherein the nitrogen is the nitrogen to which R⁶ and R⁷ are attached.

In certain embodiments, X¹ is N. In certain embodiments, X¹ is CH.

In certain embodiments of Formula (III), L² is

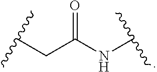 [SMILES: *CC(=O)N*]

In certain embodiments of Formula (III), L² is

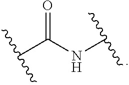 [SMILES: *NC(*)=O]

In certain embodiments of Formula (III), L² is

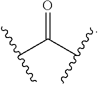 [SMILES: *C(*)=O]

In certain embodiments of Formula (III), Y is NR¹⁵. In certain embodiments of Formula (III), Y is NR¹⁵ and R¹⁵ is —C(O)R⁹. In certain embodiments of Formula (III), Y is NR¹⁵, R¹⁵ is —C(O)R⁹, and R⁹ is C₁₋₆alkyl. In certain embodiments of Formula (III), Y is NR¹⁵, R¹⁵ is —C(O)R⁹, and R⁹ is methyl. In certain embodiments of Formula (III), Y is NR¹⁵ and R¹⁵ is hydrogen. In certain embodiments of Formula (III), Y is NR¹⁵ and R¹⁵ is C₁₋₆alkyl. In certain embodiments of Formula (III), Y is NR¹⁵ and R¹⁵ is methyl. In certain embodiments of Formula (III), Y is O. In certain embodiments of Formula (III), Y is CR¹⁶R¹⁷. In certain embodiments of Formula (III), Y is CH₂.

Non-limiting examples of Formula (I) include:

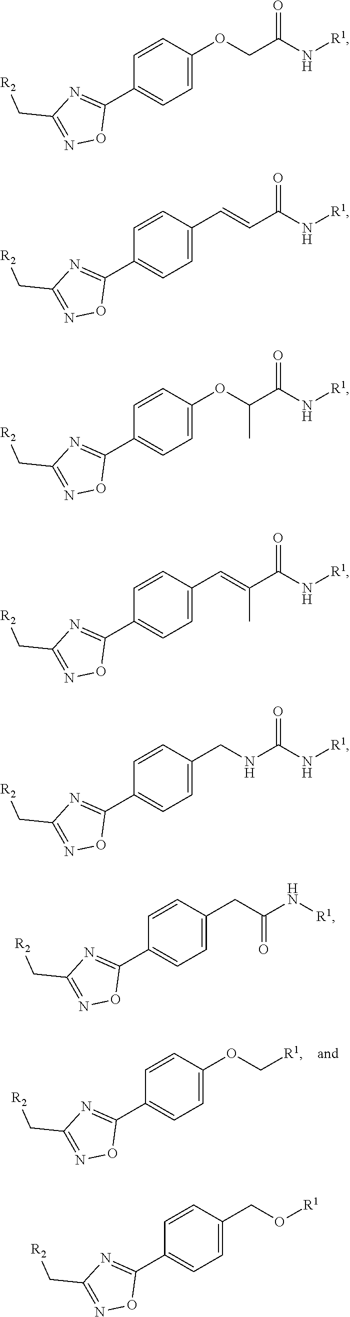

or a pharmaceutically acceptable salt, diastereomer, or stereoisomer thereof.

Non-limiting examples of Formula (II) include:

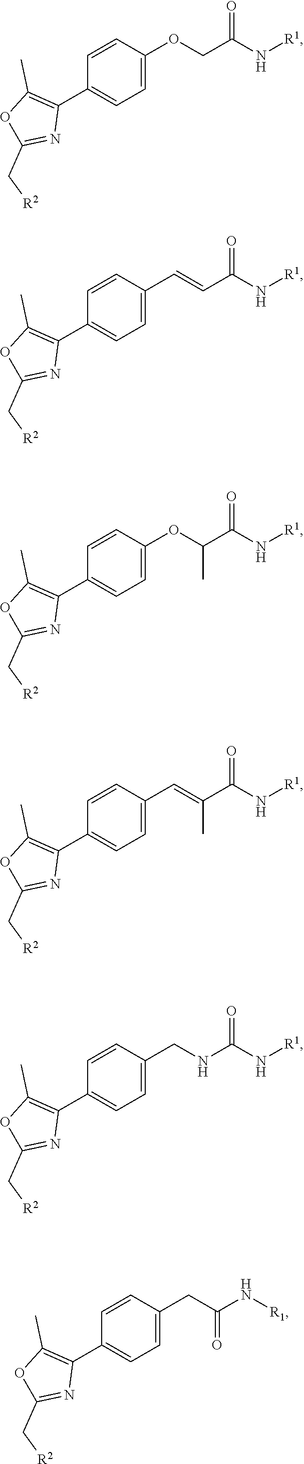

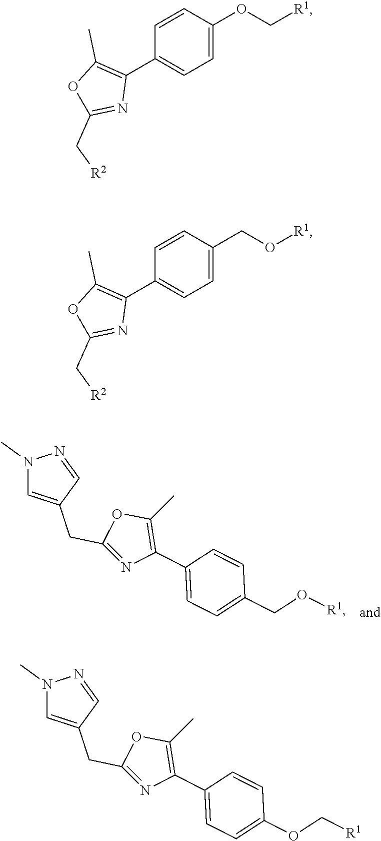

or a pharmaceutically acceptable salt, diastereomer, or stereoisomer thereof.

Non-limiting examples of Formula (Ia) include:

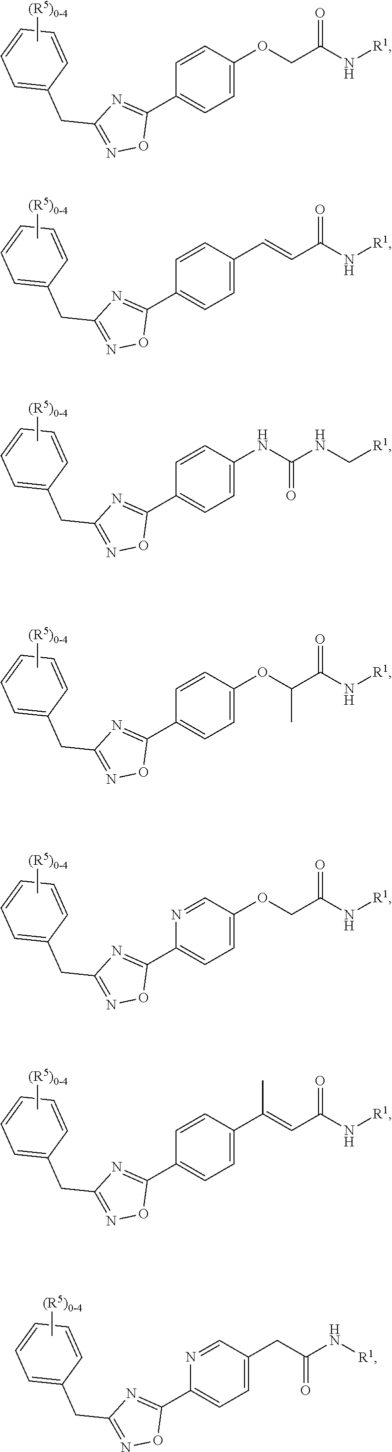

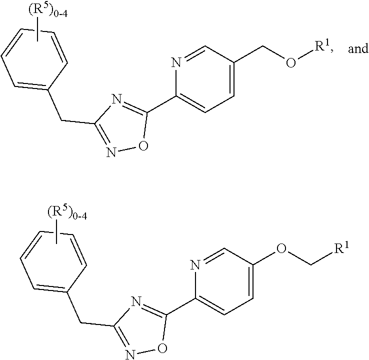

or a pharmaceutically acceptable salt, diastereomer, or stereoisomer thereof.

Non-limiting examples of Formula (Ib) include:

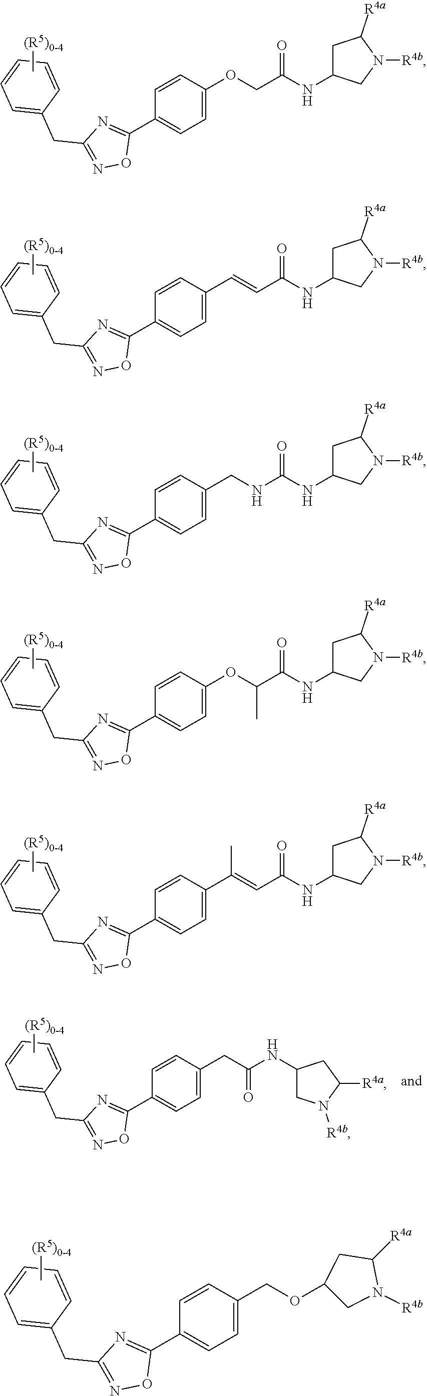

or a pharmaceutically acceptable salt, diastereomer, or stereoisomer thereof.

In certain embodiments of Formula (Ib), R^{4b} is cyano. In certain embodiments of Formula (Ib), R^{4b} is hydrogen. In certain embodiments of Formula (Ib), R^{4a} is methyl. In certain embodiments of Formula (Ib), R^{4b} is cyano and R^{4a} is methyl. In certain embodiments of Formula (Ib), R⁵ is chloro.

Non-limiting examples of Formula (Ic) or Formula (Id) include:

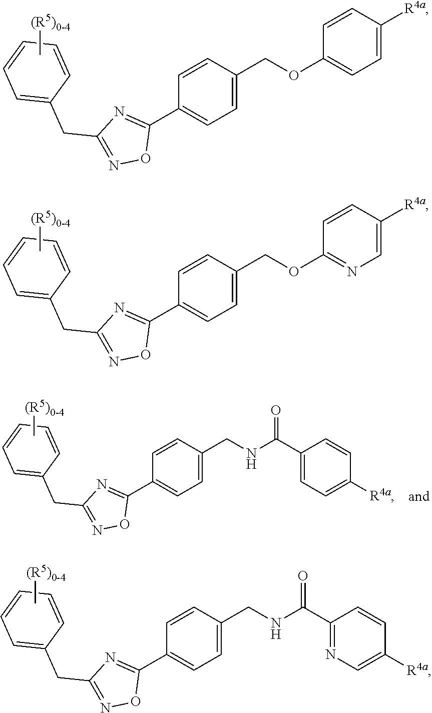

or a pharmaceutically acceptable salt, diastereomer, or stereoisomer thereof.

In certain embodiments of Formula (Ic) or Formula (Id), R¹⁰ is —C(O)NR⁶R⁷. In certain embodiments of Formula (Ic) or Formula (Id), R¹⁰ is —CH₂NR⁶R⁷. In certain embodiments of Formula (Ic) or Formula (Id), R¹⁰ is C₁₋₆alkyl. In certain embodiments of Formula (Ic) or Formula (Id), R¹⁰ is methyl.

Non-limiting examples of Formula (IIa) and (IIb) include:

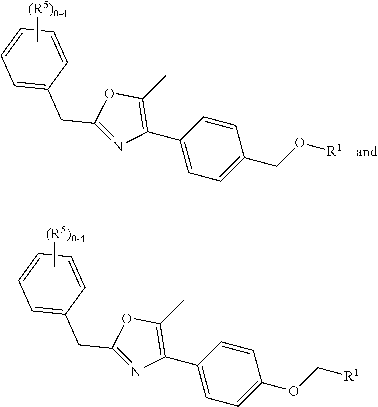

or a pharmaceutically acceptable salt, diastereomer, or stereoisomer thereof.

Non-limiting examples of Formula (IIb) include:

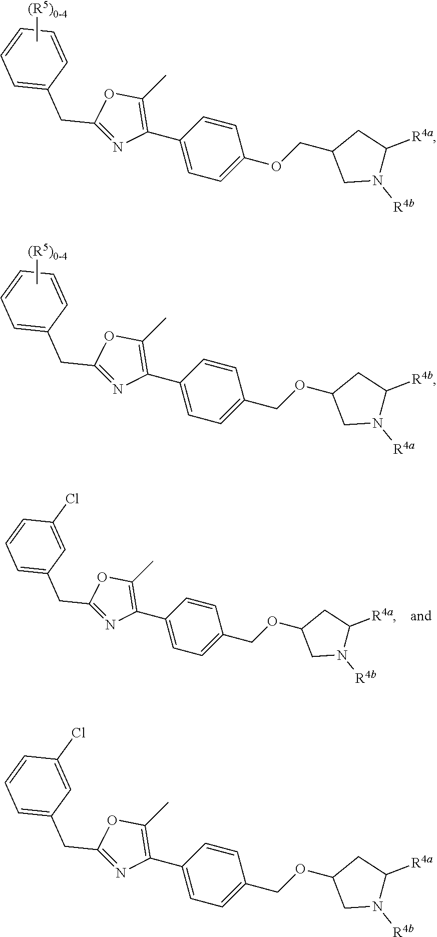

or a pharmaceutically acceptable salt, diastereomer, or stereoisomer thereof.

Non-limiting examples of Formula (IIc) or Formula (IId) include:

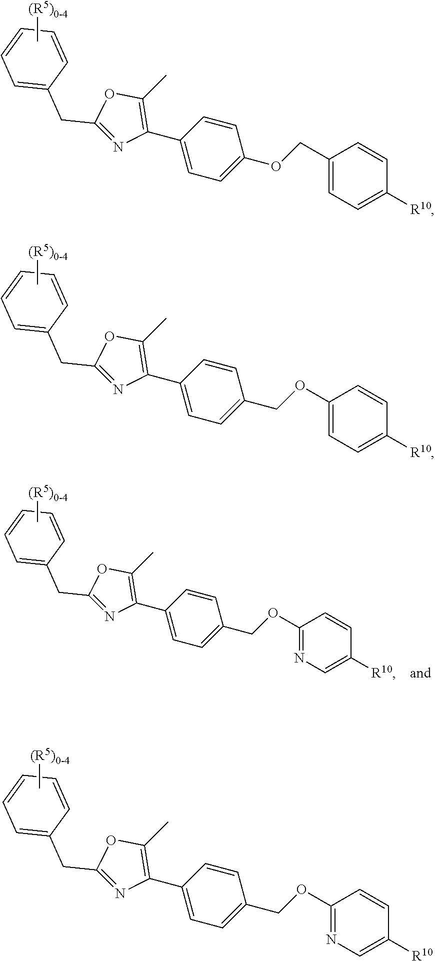

or a pharmaceutically acceptable salt, diastereomer, or stereoisomer thereof.

In certain embodiments of Formula (IIc) or Formula (IId), R¹⁰ is —C(O)NR⁶R⁷. In certain embodiments of Formula (IIc) or Formula (IId), R¹⁰ is —CH₂NR⁶R⁷. In certain embodiments of Formula (IIc) or Formula (IId), R¹⁰ is C₁₋₆alkyl. In certain embodiments of Formula (IIc) or Formula (IId), R¹⁰ is methyl.

Non-limiting examples of Formula (III) include:

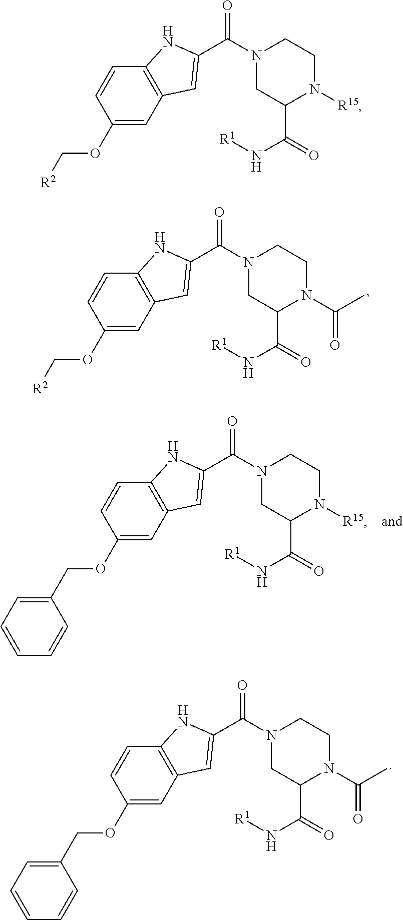

In certain embodiments, provided is a compound of Table 1 below, or a pharmaceutically salt, diastereomer, stereoisomer, and/or mixture thereof.

Structure and Name

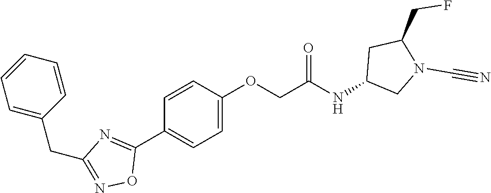 [SMILES: N#CN1C[C@H](NC(=O)COc2ccc(-c3nc(Cc4ccccc4)no3)cc2)C[C@H]1CF]

1
2-[4-(3-Benzyl-1,2,4-oxadiazol-5-yl)phenoxy]-N-[(3R,5S)-1-cyano-5-
(fluoromethyl)pyrrolidin-3-yl]acetamide

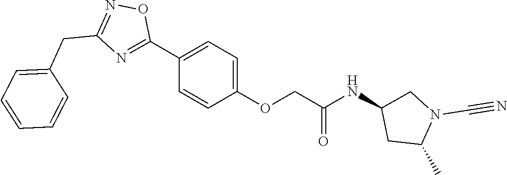 [SMILES: C[C@@H]1C[C@@H](NC(=O)COc2ccc(-c3nc(Cc4ccccc4)no3)cc2)CN1C#N]

2
2-[4-(3-Benzyl-1,2,4-oxadiazol-5-yl)phenoxy]-N-[(3R,5R)-1-cyano-5-methylpyrrolidin-3-
yl]acetamide

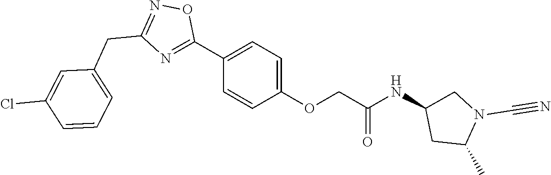 [SMILES: C[C@@H]1C[C@@H](NC(=O)COc2ccc(-c3nc(Cc4cccc(Cl)c4)no3)cc2)CN1C#N]

3
2-(4-{3-[(3-Chlorophenyl)methyl]-1,2,4-oxadiazol-5-yl}phenoxy)-N-[(3R,5R)-1-cyano-5-
methylpyrrolidin-3-yl]acetamide

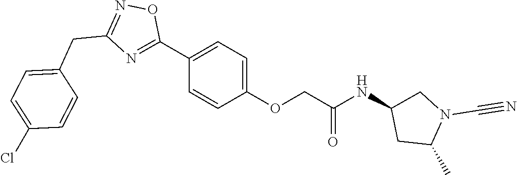 [SMILES: C[C@@H]1C[C@@H](NC(=O)COc2ccc(-c3nc(Cc4ccc(Cl)cc4)no3)cc2)CN1C#N]

4
2-(4-{3-[(4-Chlorophenyl)methyl]-1,2,4-oxadiazol-5-yl}phenoxy)-N-[(3R,5R)-1-cyano-5-
methylpyrrolidin-3-yl]acetamide

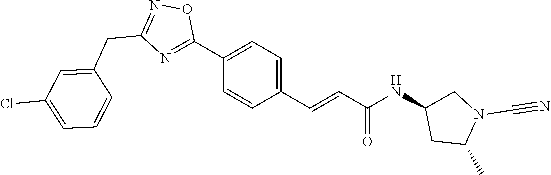 [SMILES: C[C@@H]1C[C@@H](NC(=O)/C=C/c2ccc(-c3nc(Cc4cccc(Cl)c4)no3)cc2)CN1C#N]

5
(2E)-3-(4-{3-[(3-Chlorophenyl)methyl]-1,2,4-oxadiazol-5-yl}phenyl)-N-[(3R,5R)-1-
cyano-5-methylpyrrolidin-3-yl]prop-2-enamide

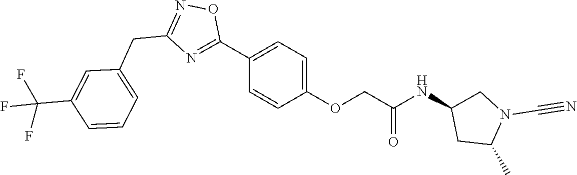 [SMILES: C[C@@H]1C[C@@H](NC(=O)COc2ccc(-c3nc(Cc4cccc(C(F)(F)F)c4)no3)cc2)CN1C#N]

6
N-[(3R,5R)-1-Cyano-5-methylpyrrolidin-3-y1]-2-[4-(3-{[3-
(trifluoromethyl)phenyl]methyl}-1,2,4-oxadiazol-5-y1)phenoxy]acetamide

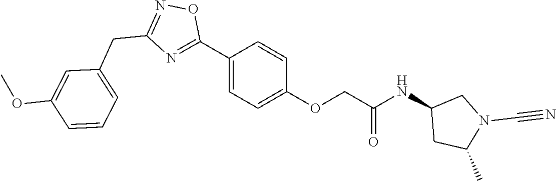 [SMILES: COc1cccc(Cc2noc(-c3ccc(OCC(=O)N[C@@H]4C[C@@H](C)N(C#N)C4)cc3)n2)c1]

7
N-[(3R,5R)-1-Cyano-5-methylpyrrolidin-3-yl]-2-(4-{3-[(3-methoxyphenyl)methyl]-1,2,4-
oxadiazol-5-yl}phenoxy)acetamide

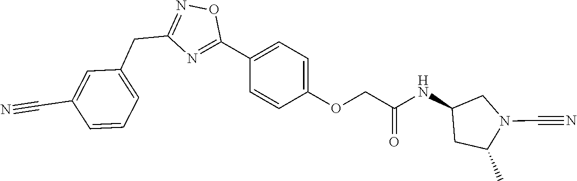 [SMILES: C[C@@H]1C[C@@H](NC(=O)COc2ccc(-c3nc(Cc4cccc(C#N)c4)no3)cc2)CN1C#N]

8
N-[(3R,5R)-1-Cyano-5-methylpyrrolidin-3-yl]-2-(4-{3-[(3-cyanophenyl)methyl]-1,2,4-
oxadiazol-5-yl}phenoxy)acetamide

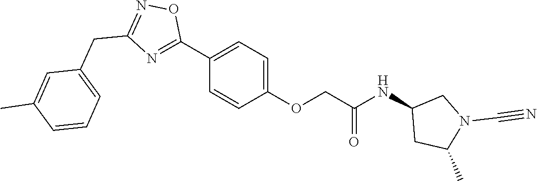 [SMILES: Cc1cccc(Cc2noc(-c3ccc(OCC(=O)N[C@@H]4C[C@@H](C)N(C#N)C4)cc3)n2)c1]

9
N-[(3R,5R)-1-Cyano-5-methylpyrrolidin-3-yl]-2-(4-{3-[(3-methylphenyl)methyl]-1,2,4-
oxadiazol-5-yl}phenoxy)acetamide

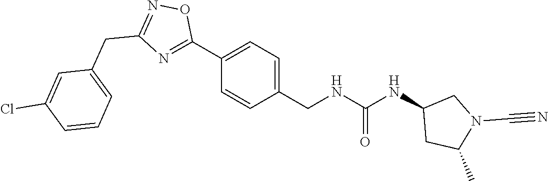 [SMILES: C[C@@H]1C[C@@H](NC(=O)NCc2ccc(-c3nc(Cc4cccc(Cl)c4)no3)cc2)CN1C#N]

10
1-[(4-{3-[(3-Chlorophenyl)methyl]-1,2,4-oxadiazol-5-yl}phenyl)methyl]-3-[(3R,5R)-1-
cyano-5-methylpyrrolidin-3-yl]urea

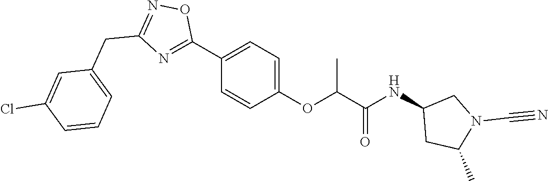 [SMILES: CC(Oc1ccc(-c2nc(Cc3cccc(Cl)c3)no2)cc1)C(=O)N[C@@H]1C[C@@H](C)N(C#N)C1]

11
2-(4-{3-[(3-Chlorophenyl)methyl]-1,2,4-oxadiazol-5-yl}phenoxy)-N-[(3R,5R)-1-cyano-5-
methylpyrrolidin-3-yl]propanamide

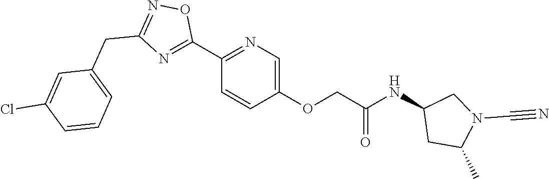 [SMILES: C[C@@H]1C[C@@H](NC(=O)COc2ccc(-c3nc(Cc4cccc(Cl)c4)no3)nc2)CN1C#N]

12
2-[(6-{3-[(3-Chlorophenyl)methyl]-1,2,4-oxadiazol-5-yl}pyridin-3-yl)oxy]-N-[(3R,5R)-1-
cyano-5-methylpyrrolidin-3-yl]acetamide

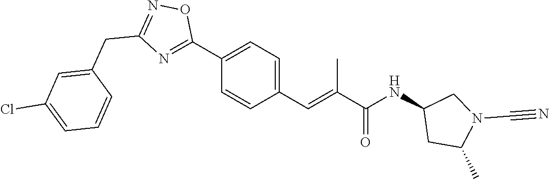 [SMILES: C/C(=C\c1ccc(-c2nc(Cc3cccc(Cl)c3)no2)cc1)C(=O)N[C@@H]1C[C@@H](C)N(C#N)C1]

13
(2E)-3-(4-{3-[(3-Chlorophenyl)methyl]-1,2,4-oxadiazol-5-yl}phenyl)-N-[(3R,5R)-1-
cyano-5-methylpyrrolidin-3-yl]-2-methylprop-2-enamide

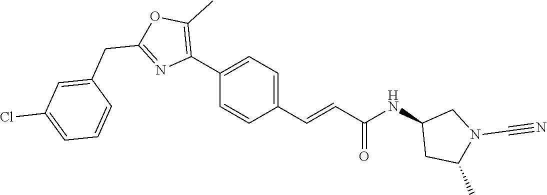 [SMILES: Cc1oc(Cc2cccc(Cl)c2)nc1-c1ccc(/C=C/C(=O)N[C@@H]2C[C@@H](C)N(C#N)C2)cc1]

14
(2E)-3-(4-{2-[(3-Chlorophenyl)methyl]-5-methyl-1,3-oxazol-4-yl}phenyl)-N-[(3R,5R)-1-
cyano-5-methylpyrrolidin-3-yl]prop-2-enamide

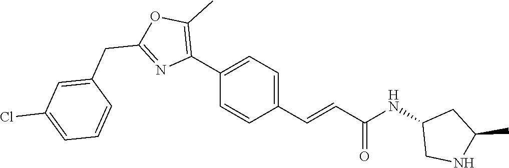 [SMILES: Cc1oc(Cc2cccc(Cl)c2)nc1-c1ccc(/C=C/C(=O)N[C@H]2CN[C@H](C)C2)cc1]

15
(2E)-3-(4-{2-[(3-Chlorophenyl)methyl]-5-methyl-1,3-oxazol-4-yl}phenyl)-N-[(3R,5R)-5-
methylpyrrolidin-3-yl]prop-2-enamide

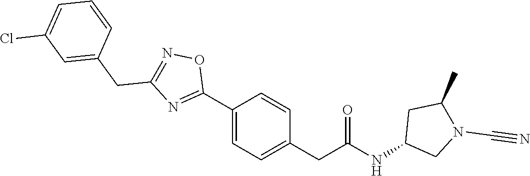 [SMILES: C[C@@H]1C[C@@H](NC(=O)Cc2ccc(-c3nc(Cc4cccc(Cl)c4)no3)cc2)CN1C#N]

16
2-(4-{3-[(3-Chlorophenyl)methyl]-1,2,4-oxadiazol-5-yl}phenyl)-N-[(3R,5R)-1-cyano-5-
methylpyrrolidin-3-yl]acetamide

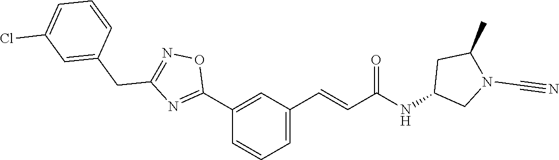 [SMILES: C[C@@H]1C[C@@H](NC(=O)/C=C/c2cccc(-c3nc(Cc4cccc(Cl)c4)no3)c2)CN1C#N]

17
(2E)-3-(3-{3-[(3-Chlorophenyl)methyl]-1,2,4-oxadiazol-5-yl}phenyl)-N-[(3R,5R)-1-
cyano-5-methylpyrrolidin-3-yl]prop-2-enamide

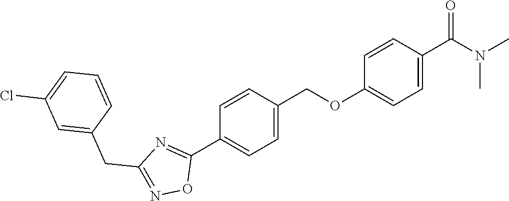 [SMILES: CN(C)C(=O)c1ccc(OCc2ccc(-c3nc(Cc4cccc(Cl)c4)no3)cc2)cc1]

18
4-[(4-{3-[(3-Chlorophenyl)methyl]-1,2,4-oxadiazol-5-yl}phenyl)methoxy]-N,N-
dimethylbenzamide

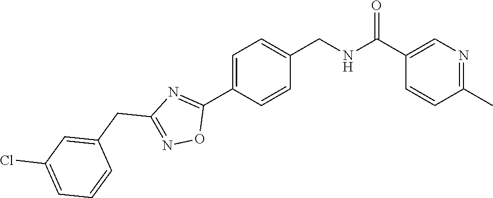 [SMILES: Cc1ccc(C(=O)NCc2ccc(-c3nc(Cc4cccc(Cl)c4)no3)cc2)cn1]

19
N-[(4-{3-[(3-Chlorophenyl)methyl]-1,2,4-oxadiazol-5-yl}phenyl)methyl]-6-
methylpyridine-3-carboxamide

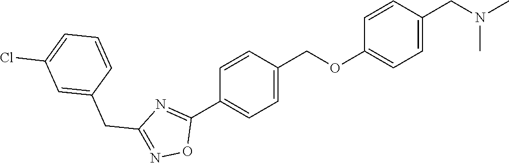 [SMILES: CN(C)Cc1ccc(OCc2ccc(-c3nc(Cc4cccc(Cl)c4)no3)cc2)cc1]

20
({4-[(4-{3-[(3-Chlorophenyl)methyl]-1,2,4-oxadiazol-5-
yl}phenyl)methoxy]phenyl}methyl)dimethylamine

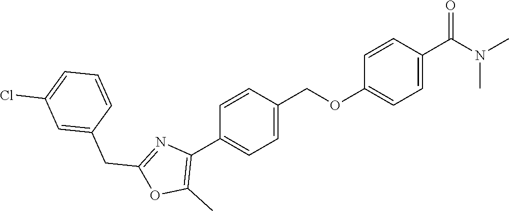 [SMILES: Cc1oc(Cc2cccc(Cl)c2)nc1-c1ccc(COc2ccc(C(=O)N(C)C)cc2)cc1]

21
4-[(4-{2-[(3-Chlorophenyl)methyl]-5-methyl-1,3-oxazol-4-yl}phenyl)methoxy]-N,N-
dimethylbenzamide

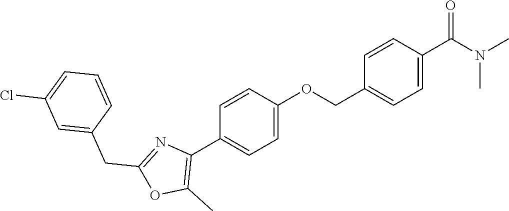 [SMILES: Cc1oc(Cc2cccc(Cl)c2)nc1-c1ccc(OCc2ccc(C(=O)N(C)C)cc2)cc1]

22
4-[(4-{2-[(3-Chlorophenyl)methyl]-5-methyl-1,3-oxazol-4-yl}phenoxy)methyl]-N,N-
dimethylbenzamide

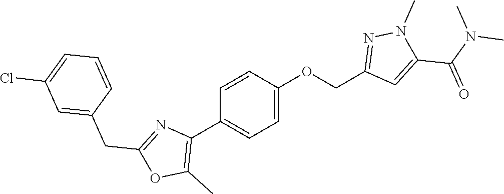 [SMILES: Cc1oc(Cc2cccc(Cl)c2)nc1-c1ccc(OCc2cc(C(=O)N(C)C)n(C)n2)cc1]

23
3-[(4-{2-[(3-Chlorophenyl)methyl]-5-methyl-1,3-oxazol-4-yl}phenoxy)methyl]-N,N,1-
trimethyl-1H-pyrazole-5-carboxamide

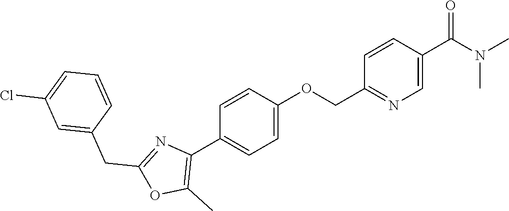 [SMILES: Cc1oc(Cc2cccc(Cl)c2)nc1-c1ccc(OCc2ccc(C(=O)N(C)C)cn2)cc1]

24
6-[(4-{2-[(3-Chlorophenyl)methyl]-5-methyl-1,3-oxazol-4-yl}phenoxy)methyl]-N,N-
dimethylpyridine-3-carboxamide

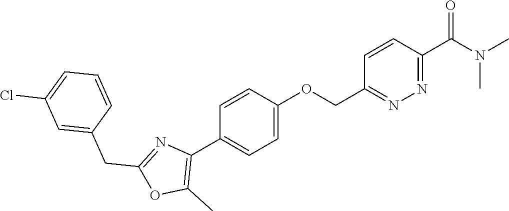 [SMILES: Cc1oc(Cc2cccc(Cl)c2)nc1-c1ccc(OCc2ccc(C(=O)N(C)C)nn2)cc1]

25
6-[(4-{2-[(3-Chlorophenyl)methyl]-5-methyl-1,3-oxazol-4-yl}phenoxy)methyl]-N,N-
dimethylpyridazine-3-carboxamide

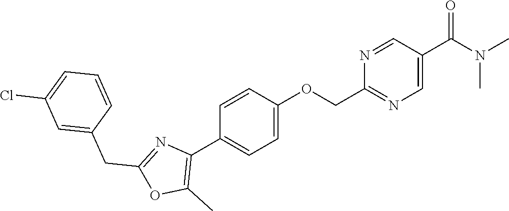 [SMILES: Cc1oc(Cc2cccc(Cl)c2)nc1-c1ccc(OCc2ncc(C(=O)N(C)C)cn2)cc1]

26
2-[(4-{2-[(3-Chlorophenyl)methyl]-5-methyl-1,3-oxazol-4-yl}phenoxy)methyl]-N,N-
dimethylpyrimidine-5-carboxamide

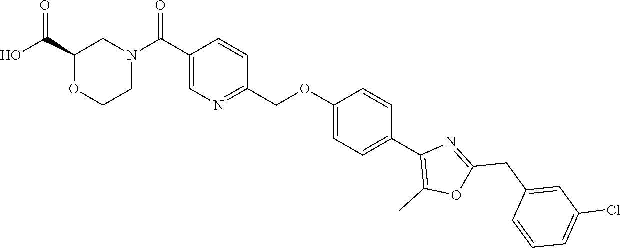 [SMILES: Cc1oc(Cc2cccc(Cl)c2)nc1-c1ccc(OCc2ccc(C(=O)N3CCO[C@@H](C(=O)O)C3)cn2)cc1]

27
(2R)-4-{6-[(4-{2-[(3-Chlorophenyl)methyl]-5-methyl-1,3-oxazol-4-
yl}phenoxy)methyl]pyridine-3-carbonyl}morpholine-2-carboxylic acid

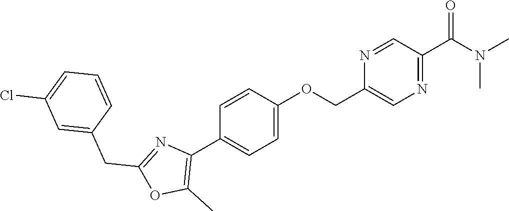 [SMILES: Cc1oc(Cc2cccc(Cl)c2)nc1-c1ccc(OCc2cnc(C(=O)N(C)C)cn2)cc1]

28
5-[(4-{2-[(3-Chlorophenyl)methyl]-5-methyl-1,3-oxazol-4-yl}phenoxy)methyl]-N,N-
dimethylpyrazine-2-carboxamide

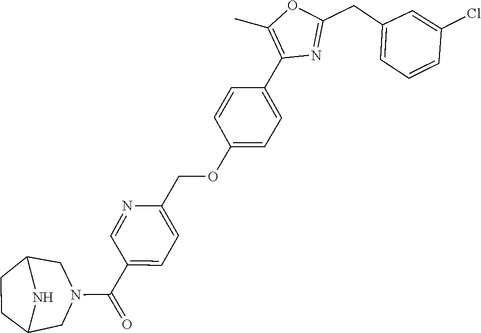 [SMILES: Cc1oc(Cc2cccc(Cl)c2)nc1-c1ccc(OCc2ccc(C(=O)N3CC4CCC(C3)N4)cn2)cc1]

29
3-{6-[(4-{2-[(3-Chlorophenyl)methyl]-5-methyl-1,3-oxazol-4-
yl}phenoxy)methyl]pyridine-3-carbonyl}-3,8-diazabicyclo[3.2.1]octane

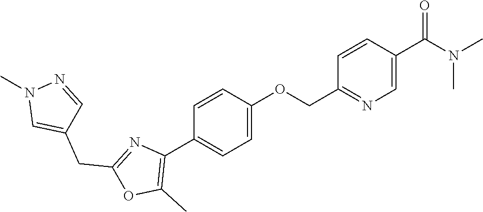

30
N,N-Dimethyl-6-[(4-{5-methyl-2-[(1-methyl-1H-pyrazol-4-yl)methyl]-1,3-oxazol-4-
yl}phenoxy)methyl]pyridine-3-carboxamide

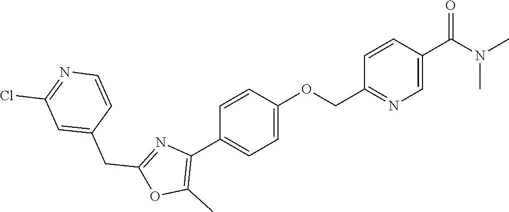 [SMILES: Cc1oc(Cc2ccnc(Cl)c2)nc1-c1ccc(OCc2ccc(C(=O)N(C)C)cn2)cc1]

31
6-[(4-{2-[(2-Chloropyridin-4-yl)methyl]-5-methyl-1,3-oxazol-4-yl}phenoxy)methyl]-N,N-
dimethylpyridine-3-carboxamide

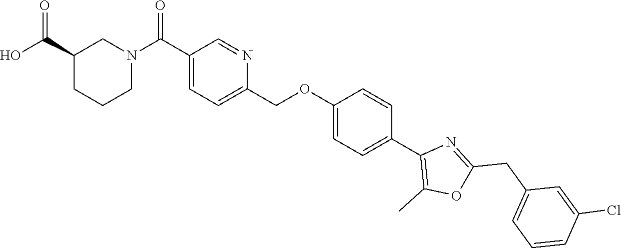 [SMILES: Cc1oc(Cc2cccc(Cl)c2)nc1-c1ccc(OCc2ccc(C(=O)N3CCC[C@@H](C(=O)O)C3)cn2)cc1]

32
(3R)-1-{6-[(4-{2-[(3-Chlorophenyl)methyl]-5-methyl-1,3-oxazol-4-
yl}phenoxy)methyl]pyridine-3-carbonyl}piperidine-3-carboxylic acid

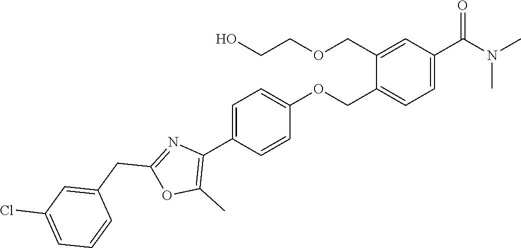 [SMILES: Cc1oc(Cc2cccc(Cl)c2)nc1-c1ccc(OCc2ccc(C(=O)N(C)C)cc2COCCO)cc1]

33
4-[(4-{2-[(3-Chlorophenyl)methyl]-5-methyl-1,3-oxazol-4-yl}phenoxy)methyl]-3-[(2-
hydroxyethoxy)methyl]-N,N-dimethylbenzamide

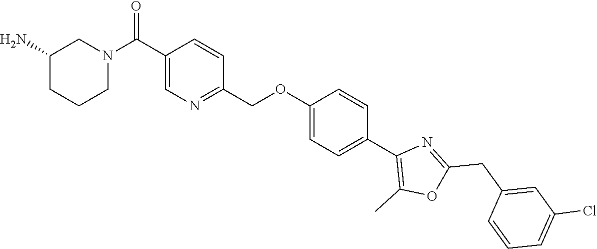 [SMILES: Cc1oc(Cc2cccc(Cl)c2)nc1-c1ccc(OCc2ccc(C(=O)N3CCC[C@H](N)C3)cn2)cc1]

34
(3S)-1-{6-[(4-{2-[(3-Chlorophenyl)methyl]-5-methyl-1,3-oxazol-4-
yl}phenoxy)methyl]pyridine-3-carbonyl}piperidin-3-amine

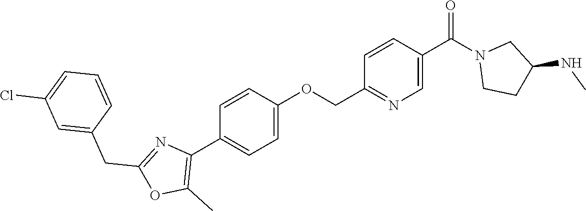 [SMILES: CN[C@H]1CCN(C(=O)c2ccc(COc3ccc(-c4nc(Cc5cccc(Cl)c5)oc4C)cc3)nc2)C1]

35
(3S)-1-{6-[(4-{2-[(3-Chlorophenyl)methyl]-5-methyl-1,3-oxazol-4-
yl}phenoxy)methyl]pyridine-3-carbonyl}-N-methylpyrrolidin-3-amine

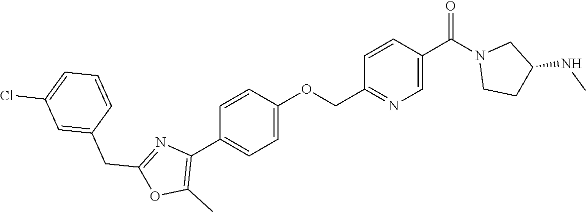 [SMILES: CN[C@@H]1CCN(C(=O)c2ccc(COc3ccc(-c4nc(Cc5cccc(Cl)c5)oc4C)cc3)nc2)C1]

36
(3R)-1-{6-[(4-{2-[(3-Chlorophenyl)methyl]-5-methyl-1,3-oxazol-4-
yl}phenoxy)methyl]pyridine-3-carbonyl}-N-methylpyrrolidin-3-amine

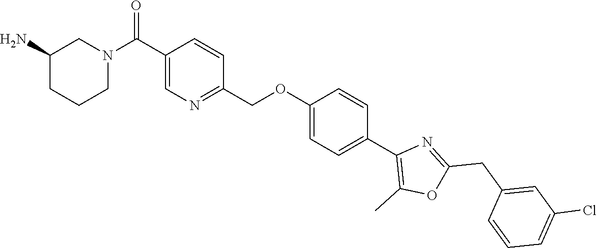 [SMILES: Cc1oc(Cc2cccc(Cl)c2)nc1-c1ccc(OCc2ccc(C(=O)N3CCC[C@@H](N)C3)cn2)cc1]

37
(3R)-1-{6-[(4-{2-[(3-Chlorophenyl)methyl]-5-methyl-1,3-oxazol-4-
yl}phenoxy)methyl]pyridine-3-carbonyl}piperidin-3-amine

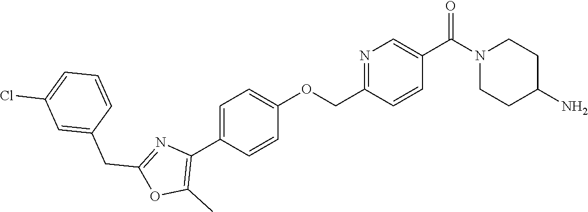 [SMILES: Cc1oc(Cc2cccc(Cl)c2)nc1-c1ccc(OCc2ccc(C(=O)N3CCC(N)CC3)cn2)cc1]

38
1-{6-[(4-{2-[(3-Chlorophenyl)methyl]-5-methyl-1,3-oxazol-4-
yl}phenoxy)methyl]pyridine-3-carbonyl}piperidin-4-amine

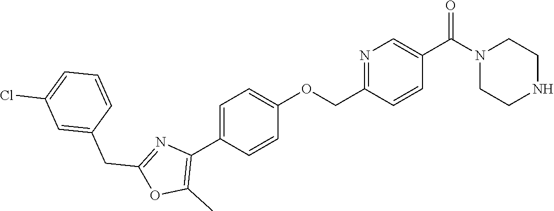 [SMILES: Cc1oc(Cc2cccc(Cl)c2)nc1-c1ccc(OCc2ccc(C(=O)N3CCNCC3)cn2)cc1]

39
1-{6-[(4-{2-[(3-Chlorophenyl)methyl]-5-methyl-1,3-oxazol-4-
yl}phenoxy)methyl]pyridine-3-carbonyl}piperazine

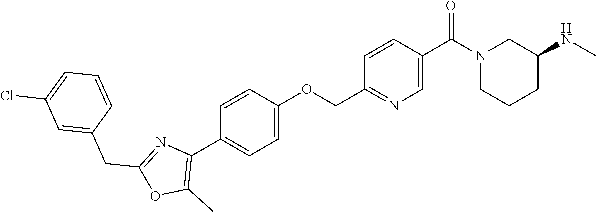 [SMILES: CN[C@H]1CCCN(C(=O)c2ccc(COc3ccc(-c4nc(Cc5cccc(Cl)c5)oc4C)cc3)nc2)C1]

40
(3S)-1-{6-[(4-{2-[(3-Chlorophenyl)methyl]-5-methyl-1,3-oxazol-4-
yl}phenoxy)methyl]pyridine-3-carbonyl}-N-methylpiperidin-3-amine

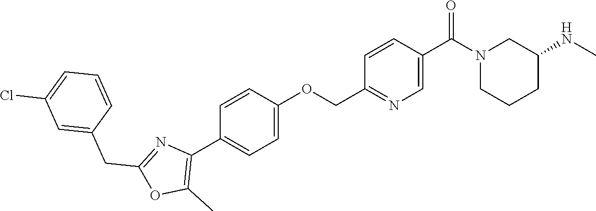 [SMILES: CN[C@@H]1CCCN(C(=O)c2ccc(COc3ccc(-c4nc(Cc5cccc(Cl)c5)oc4C)cc3)nc2)C1]

41
(3R)-1-{6-[(4-{2-[(3-Chlorophenyl)methyl]-5-methyl-1,3-oxazol-4-
yl}phenoxy)methyl]pyridine-3-carbonyl}-N-methylpiperidin-3-amine

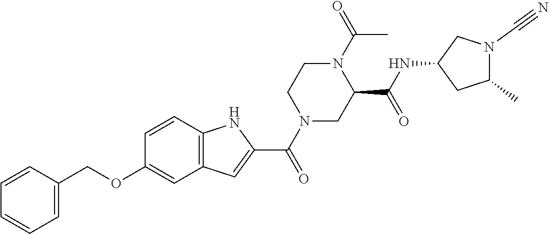 [SMILES: CC(=O)N1CCN(C(=O)c2cc3cc(OCc4ccccc4)ccc3[nH]2)C[C@@H]1C(=O)N[C@H]1C[C@@H](C)N(C#N)C1]

42
(2R)-1-Acetyl-4-[5-(benzyloxy)-1H-indole-2-carbonyl]-N-[(3S,5R)-1-cyano-5-
methylpyrrolidin-3-yl]piperazine-2-carboxamide

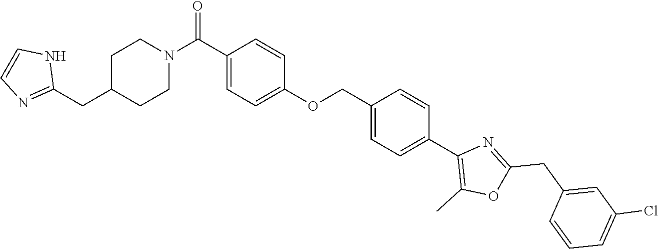 [SMILES: Cc1oc(Cc2cccc(Cl)c2)nc1-c1ccc(COc2ccc(C(=O)N3CCC(Cc4ncc[nH]4)CC3)cc2)cc1]

43
1-{4-[(4-{2-[(3-Chlorophenyl)methyl]-5-methyl-1,3-oxazol-4-
yl}phenyl)methoxy]benzoyl}-4-[(1H-imidazol-2-yl)methyl]piperidine

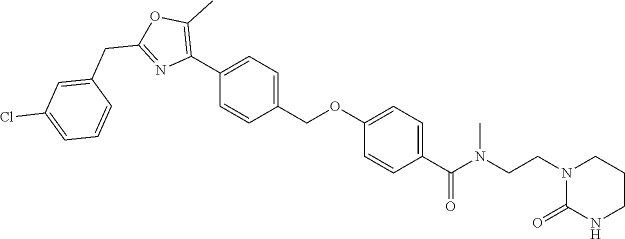 [SMILES: Cc1oc(Cc2cccc(Cl)c2)nc1-c1ccc(COc2ccc(C(=O)N(C)CCN3CCCNC3=O)cc2)cc1]

44
4-[(4-{2-[(3-Chlorophenyl)methyl]-5-methyl-1,3-oxazol-4-yl}phenyl)methoxy]-N-methyl-
N-[2-(2-oxo-1,3-diazinan-1-yl)ethyl]benzamide

### Optically Active Compounds

In certain embodiments, compounds provided herein may have several chiral centers and may exist in and be isolated in optically active and racemic forms. In certain embodiments, some compounds may exhibit polymorphism. A person of skill in the art will appreciate that compounds provided herein can exist in any racemic, optically-active, diastereomeric, polymorphic, or stereoisomeric form, and/or mixtures thereof. A person of skill in the art will also appreciate that such compounds described herein that possess the useful properties also described herein are within the scope of this disclosure. A person of skill in the art will further appreciate how to prepare optically active forms of the compounds described herein, for example, by resolution of racemic forms via recrystallization techniques, by synthesis from optically-active starting materials, by chiral synthesis, or by chromatographic separation using a chiral stationary phase. In addition, most amino acids are chiral (i.e., designated as L- or D-, wherein the L-enantiomer is the naturally occurring configuration) and can exist as separate enantiomers.

Examples of methods to obtain optically active materials are known in the art, and include at least the following:
i) physical separation of crystals—a technique whereby macroscopic crystals of the individual enantiomers are manually separated. This technique can be used if crystals of the separate enantiomers exist (i.e., the material is a conglomerate, and the crystals are visually distinct);
ii) simultaneous crystallization—a technique whereby the individual enantiomers are separately crystallized from a solution of the racemate, only if the latter is a conglomerate in the solid state;
iii) enzymatic resolutions—a technique wherein partial, or complete separation of a racemate is accomplished by virtue of different rates of reaction of the enantiomers in the presence of an enzyme;
iv) enzymatic asymmetric synthesis—a synthetic technique wherein at least one step of the synthesis uses an enzymatic reaction to obtain an enantiomerically pure, or enriched synthetic precursor of the desired enantiomer;
v) chemical asymmetric synthesis—a synthetic technique wherein the desired enantiomer is synthesized from an achiral precursor using chiral catalysts, or chiral auxiliaries to produce asymmetry (i.e., chirality) in the product;
vi) diastereomer separations—a technique wherein a racemic compound is treated with an enantiomerically pure reagent (a chiral auxiliary) that converts the individual enantiomers to diastereomers. The resulting diastereomers are then separated by chromatography, or crystallization by virtue of their now more distinct diastereomeric differences, and then the chiral auxiliary is removed to obtain each enantiomer;
vii) first- and second-order asymmetric transformations—a technique wherein diastereomers of the racemate equilibrate in solution to yield a preponderance of a diastereomer of the desired enantiomer, or where kinetic, or thermodynamic crystallization of the diastereomer of the desired enantiomer perturbs the equilibrium such that eventually in principle all the material is converted to the crystalline diastereomer of the desired enantiomer. The desired enantiomer is then derived from the diastereomer;
viii) kinetic resolutions—this technique refers to the achievement of partial or complete resolution of a racemate (or of a further resolution of a partially resolved compound) by virtue of unequal reaction rates of the enantiomers with a chiral, or non-racemic reagent, or catalyst under kinetic conditions;
ix) enantiospecific synthesis from non-racemic precursors—a synthetic technique wherein the desired enantiomer is obtained from chiral starting materials, and where the stereochemical integrity is not or is only minimally compromised over the course of the synthesis;
x) chiral liquid chromatography—a technique wherein the enantiomers of a racemate are separated in a liquid mobile phase by virtue of their different interactions with a stationary phase. The stationary phase can be made of chiral material, or the mobile phase can contain an additional chiral material to provoke the different interactions;
xi) chiral gas chromatography—a technique wherein the racemate is volatilized and enantiomers are separated by virtue of their different interactions in the gaseous mobile phase with a column containing a fixed non-racemic adsorbent phase;
xii) extraction with chiral solvents—a technique wherein the enantiomers are separated by virtue of kinetic or thermodynamic dissolution of one enantiomer into a particular chiral solvent;
xiii) transport across chiral membranes—a technique wherein a racemate is placed in contact with a thin membrane barrier. The barrier typically separates two miscible fluids, one containing the racemate, and a driving force such as a concentration, or pressure differential causes preferential transport across the membrane barrier. Separation occurs as a result of the non-racemic nature of the membrane which allows only one enantiomer of the racemate to pass through.

In some embodiments, provided herein are compositions of compounds of any of Formulas (I)—(III), that are substantially free of a designated stereoisomer of that compound. In certain embodiments, in the methods and compounds of this disclosure, the compounds are substantially free of other stereoisomers, including the other diastereomer(s). In some embodiments, the composition includes a compound that is at least 85%, 90%, 95%, 98%, or 99% to 100% by weight of the compound, the remainder comprising other chemical species, or enantiomers. In some embodiments, provided herein are compositions of compounds of any of Formulas (I)—(III), that are substantially free of a designated enantiomer of that compound. In certain embodiments, in the methods and compounds of this disclosure, the compounds are substantially free of other enantiomers. In some embodiments, the composition includes a compound that is at least 85%, 90%, 95%, 98%, or 99% to 100% by weight of the compound, the remainder comprising other chemical species or enantiomers.

### Isotopically Enriched Compounds

Also provided herein are isotopically enriched compounds including, but not limited to, isotopically enriched compounds of any of Formulas (I)—(III).

Isotopic enrichment (for example, deuteration) of pharmaceuticals to improve pharmacokinetics (“PK”), pharmacodynamics (“PD”), and/or toxicity profiles, has been previously demonstrated within some classes of drugs. See, for example, Lijinsky et al., Food Cosmet. Toxicol., 20: 393 (1982); Lijinsky et al., J. Nat. Cancer Inst., 69: 1127 (1982); Mangold et al., Mutation Res. 308: 33 (1994); Gordon et al., Drug Metab. Dispos., 15: 589 (1987); Zello et al., Metabolism, 43: 487 (1994); Gately et al., J. Nucl. Med., 27: 388 (1986); Wade D, Chem. Biol. Interact. 117: 191 (1999).

Isotopic enrichment of a drug can be used, for example, to (1) reduce or eliminate unwanted metabolites; (2) increase the half-life of the parent drug; (3) decrease the number of doses needed to achieve a desired effect; (4) decrease the amount of a dose necessary to achieve a desired effect; (5) increase the formation of active metabolites if any are formed; and/or (6) decrease the production of deleterious metabolites in specific tissues. Isotopic enrichment of a drug can also be used to create a more effective and/or safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e., the step with the highest transition state energy), substitution of a (heavier) isotope for that reactive hydrogen will cause a decrease in the reaction rate. The Deuterium Kinetic Isotope Effect (“DKIE”) is the most common form of KIE. (See, e.g., Foster et al., Adv. Drug Res., vol. 14, pp. 1-36 (1985); Kushner et al., Can. J. Physiol. Pharmacol., vol. 77, pp. 79-88 (1999)).

The magnitude of the DKIE can be expressed as the ratio between the rates of a given reaction in which a C—H bond is broken, and the same reaction where deuterium is substituted for hydrogen and the C-D bond is broken. The DKIE can range from about one (no isotope effect) to very large numbers, such as 50, or more, meaning that the reaction can be fifty, or more, times slower when deuterium has been substituted for hydrogen.

Substitution of tritium (“T”) for hydrogen results in yet a stronger bond than deuterium and gives numerically larger isotope effects. Similarly, substitution of isotopes for other elements including, but not limited to, ¹³C, or ¹⁴C for carbon; ³³S, ³⁴S, or ³⁶S for sulfur; ¹⁵N for nitrogen; and ¹⁷O, or ¹⁸O for oxygen may lead to a similar kinetic isotope effect.

The animal body expresses a variety of enzymes for the purpose of eliminating foreign substances, such as therapeutic agents, from its circulation system. Examples of such enzymes include the cytochrome P450 enzymes (“CYPs”), esterases, proteases, reductases, dehydrogenases, and monoamine oxidases to react with and convert these foreign substances to more polar intermediates, or metabolites for renal excretion. Some of the most common metabolic reactions of pharmaceutical compounds involve the oxidation of a carbon-hydrogen (C—H) bond to either a carbon-oxygen (C—O), or carbon-carbon (C═C) pi-bond. The resultant metabolites may be stable, or unstable under physiological conditions, and can have substantially different PK/PD, and acute, and long-term toxicity profiles relative to the parent compounds. For many drugs, such oxidations are rapid. Therefore, these drugs often require the administration of multiple or high daily doses.

Therefore, isotopic enrichment at certain positions of a compound provided herein will produce a detectable KIE that will affect the pharmacologic, PK, PD, and/or toxicological profiles of a compound provided herein in comparison with a similar compound having a natural isotopic composition.

### Compositions and Uses

### Pharmaceutical Compositions and Methods of Administration

The compounds provided herein can be formulated into pharmaceutical compositions using methods available in the art and those disclosed herein. Any of the compounds provided herein can be provided in the appropriate pharmaceutical composition and be administered by a suitable route of administration.

The methods provided herein encompass administering pharmaceutical compositions comprising at least one compound provided herein and one or more compatible and pharmaceutically acceptable carriers. In this context, the term “pharmaceutically acceptable” means approved by a regulatory agency of the Federal, or state government, or listed in the U.S. Pharmacopeia, or other generally recognized pharmacopeia for use in animals, and in certain embodiments in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils including petroleum, animal, vegetable, or oils of synthetic origin, such as peanut oil, soybean oil, mineral oil, sesame oil, and the like. Water can be used as a carrier when the pharmaceutical composition is administered intravenously. Saline solutions, and aqueous dextrose, and glycerol solutions can also be employed as liquid carriers, particularly for injectable solutions. Examples of suitable pharmaceutical carriers are described in Martin, E. W., Remington's Pharmaceutical Sciences.

In clinical practice the pharmaceutical compositions, or compounds provided herein may be administered by any route known in the art. Exemplary routes of administration include, but are not limited to, inhalation, intraarterial, intradermal, intramuscular, intraperitoneal, intravenous, nasal, parenteral, pulmonary, oral, and subcutaneous routes. In some embodiments, a pharmaceutical composition or compound provided herein is administered parenterally. In some embodiments, a pharmaceutical composition or compound provided herein is administered orally.

The compositions for parenteral administration can be emulsions or sterile solutions. Parenteral compositions may include, for example, propylene glycol, polyethylene glycol, vegetable oils, and injectable organic esters (e.g., ethyl oleate). These compositions can also contain wetting, isotonizing, emulsifying, dispersing, and stabilizing agents. Sterilization can be carried out in several ways, for example, using a bacteriological filter, via radiation, or via heating. Parenteral compositions can also be prepared in the form of sterile solid compositions which can be dissolved at the time of use in sterile water, or any other injectable sterile medium.

In some embodiments, a composition provided herein is a pharmaceutical composition, or a single unit dosage form. Pharmaceutical compositions, and single unit dosage forms provided herein comprise a prophylactically, or therapeutically effective amount of one, or more prophylactic, or therapeutic compounds.

The pharmaceutical composition may comprise one, or more pharmaceutical excipients. Any suitable pharmaceutical excipient may be used, wherein a person of ordinary skill in the art is capable of selecting suitable pharmaceutical excipients. Non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. Whether a particular excipient is suitable for incorporation into a pharmaceutical composition, or dosage form depends on a variety of factors well known in the art including, but not limited to, the way in which the dosage form will be administered to a subject and the specific compound in the dosage form. The composition, or single unit dosage form, if desired, can also contain minor amounts of wetting, or emulsifying agents, or pH buffering agents. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference herein in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long-chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a co-solvent. Illustrative examples of co-solvents include, ethanol, poly(ethylene) glycol, butylene glycol, dimethylacetamide, glycerin, and propylene glycol.

In some embodiments, the pharmaceutical composition comprises a buffer. Illustrative examples of buffers include, acetate, borate, carbonate, lactate, malate, phosphate, citrate, hydroxide, diethanolamine, monoethanolamine, glycine, methionine, guar gum, and monosodium glutamate.

In some embodiments, the pharmaceutical composition comprises a carrier, or filler. Illustrative examples of carriers, or fillers include, lactose, maltodextrin, mannitol, sorbitol, chitosan, stearic acid, xanthan gum, and guar gum.

In some embodiments, the pharmaceutical composition comprises a surfactant. Illustrative examples of surfactants, include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene(glycol) succinate.

In some embodiments, the pharmaceutical composition comprises an anti-caking agent. Illustrative examples of anti-caking agents include, calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press, incorporated by reference herein in its entirety.

In some embodiments, the pharmaceutical composition comprises a solvent. In some aspects, the solvent is saline solution, such as a sterile isotonic saline solution, or dextrose solution. In some aspects, the solvent is water for injection.

In some embodiments, the pharmaceutical compositions are in a particulate form, such as a microparticle or a nanoparticle. Microparticles, and nanoparticles may be formed from any suitable material, such as a polymer, or a lipid. In some aspects, the microparticles, or nanoparticles are micelles, liposomes, or polymersomes.

Further provided herein are anhydrous pharmaceutical compositions, and dosage forms comprising a compound, since, in some embodiments, water can facilitate the degradation of some compounds.

Anhydrous pharmaceutical compositions, and dosage forms provided herein can be prepared using anhydrous, or low moisture containing ingredients, and low moisture, or low humidity conditions. Pharmaceutical compositions, and dosage forms that comprise lactose, and at least one active ingredient that comprises a primary, or secondary amine can be anhydrous if substantial contact with moisture, and/or humidity during manufacturing, packaging, and/or storage is expected.

An anhydrous pharmaceutical composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Lactose-free compositions provided herein can comprise excipients that are well known in the art and are listed, for example, in the U.S. Pharmocopeia (USP) SP (XXI)/NF (XVI). In general, lactose-free compositions comprise an active ingredient, a binder/filler, and a lubricant in pharmaceutically compatible, and pharmaceutically acceptable amounts. Exemplary lactose-free dosage forms comprise an active ingredient, microcrystalline cellulose, pre gelatinized starch, and magnesium stearate.

Also provided are pharmaceutical compositions, and dosage forms that comprise one, or more excipients that reduce the rate by which a compound will decompose. Such excipients, which are referred to herein as “stabilizers,” include, but are not limited to, antioxidants such as ascorbic acid, pH buffers, or salt buffers.

### Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically sterile, or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to Water for Injection USP; aqueous vehicles such as, but not limited to, Sodium Chloride Injection, Ringer's Injection, Dextrose Injection, Dextrose, and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one, or more of the antibodies disclosed herein can also be incorporated into the parenteral dosage forms.

### Oral Dosage Forms

In certain embodiments, provided are oral dosage forms. The compounds described herein can be formulated using any desired techniques including formulating the compound as a neat chemical (for example a powder, morphic form, amorphous form, or oil), or mixing the compound with a pharmaceutically acceptable excipient. The resulting pharmaceutically acceptable composition for oral delivery contains an effective amount of the compound or a pharmaceutically acceptable salt thereof and one or more pharmaceutically acceptable excipients.

Typical dosage forms for oral administration include a pill, a tablet, a capsule, a gel cap, a solution, a suspension, or an emulsion. The dosage form may also feature compartmentalization. For example, when the dosage form is a pill, tablet, or capsule, it may have different layers of material which have different excipients or different concentrations of excipients. For example, an enteric coated oral tablet may be used to enhance bioavailability of the compounds for an oral route of administration. The entric coating will be a layer of excipient that allows the tablet to survive stomach acid. In certain embodiments the oral dosage form contains one or more additional active agents as described herein. In certain embodiments the second active agent is administered separately from the compound of the present invention.

In another embodiment one dosage form may be converted to another to favorably improve the properties. For example, when making a solid pharmaceutically acceptable composition a suitable liquid formulation can be lyophilization. The solid can be reconstituted with an appropriate carrier or diluent prior to administration. Oral pharmaceutical compositions can contain any amount of active compound that achieves the desired result, for example between 0.1 and 99 weight % (wt. %) of the compound and usually at least about 5 wt. % of the compound.

Some embodiments contain at least about 10%, 15%, 20%, 25 wt. % to about 50 wt. % or from about 5 wt. % to about 75 wt. % of the compound. The oral dosage form can be administered, for example, once a day (q.d.), twice a day (b.i.d.), three times a day (t.i.d.), four times a day (q.i.d.), once every other day (Q2d), once every third day (Q3d), as needed, or any dosage schedule that provides treatment of a disorder described herein.

### Dosage and Unit Dosage Forms

In human therapeutics, the doctor will determine the posology which he considers most appropriate according to a preventive, or curative treatment, and according to the age, weight, condition, and other factors specific to the subject to be treated.

In certain embodiments, a composition provided herein is a pharmaceutical composition, or a single unit dosage form. Pharmaceutical compositions, and single unit dosage forms provided herein comprise a prophylactically, or therapeutically effective amount of one, or more prophylactic, or therapeutic antibodies, or antigen binding fragments thereof.

The amount of the compound or composition which will be effective in the prevention, or treatment of a disorder, or one, or more symptoms thereof will vary with the nature, and severity of the disease, or condition, and the route by which the compound is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro, or animal model test systems.

In certain embodiments, exemplary doses of a composition include milligram, or microgram amounts of the compound per kilogram of subject, or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). In certain embodiments, the dosage of the compound provided herein, based on weight of the compound, administered to prevent, treat, manage, or ameliorate a disorder, or one, or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the composition, or a composition provided herein administered to prevent, treat, manage, or ameliorate a disorder, or one, or more symptoms thereof in a subject is 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.25 mg to 2.5 mg, 0.5 mg to 20 mg, 0.5 to 15 mg, 0.5 to 12 mg, 0.5 to 10 mg, 0.5 mg to 7.5 mg, 0.5 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

The dose can be administered according to a suitable schedule, for example, once, two times, three times, or four times weekly. It may be necessary to use dosages of the compound outside the ranges disclosed herein in some cases, as will be apparent to those of ordinary skill in the art. Furthermore, it is noted that the clinician, or treating physician will know how, and when to interrupt, adjust, or terminate therapy in conjunction with subject response.

Different therapeutically effective amounts may be applicable for different diseases, and conditions, as will be readily known by those of ordinary skill in the art. Similarly, amounts sufficient to prevent, manage, treat, or ameliorate such disorders, but insufficient to cause, or sufficient to reduce, adverse effects associated with the compounds provided herein are also encompassed by the described dosage amounts, and dose frequency schedules herein. Further, when a subject is administered multiple dosages of a composition provided herein, not all of the dosages need be the same. For example, the dosage administered to the subject may be increased to improve the prophylactic, or therapeutic effect of the composition, or it may be decreased to reduce one, or more side effects that a particular subject is experiencing.

In certain embodiments, treatment, or prevention can be initiated with one, or more loading doses of a compound, or composition provided herein followed by one, or more maintenance doses.

In certain embodiments, a dose of a compound, or composition provided herein can be administered to achieve a steady-state concentration of the compound in blood, or serum of the subject. The steady-state concentration can be determined by measurement according to techniques available to those of skill or can be based on the physical characteristics of the subject such as height, weight, and age.

In certain embodiments, administration of the same composition may be repeated and the administrations may be separated by at least one day, two days, three days, five days, ten days, fifteen days, thirty days, forty-five days, two months, seventy-five days, three months, or six months.

### Therapeutic Applications

For therapeutic applications, the compounds are administered to a mammal, in certain embodiments, a human, in a pharmaceutically acceptable dosage suitable for administration form such as those known in the art, and those discussed herein, intravenously as a bolus or by continuous infusion over a period of time, by intramuscular, intraperitoneal, intra-cerebrospinal, subcutaneous, intra-articular, intrasynovial, intrathecal, or intratumoral routes. The compounds also are suitably administered by peritumoral, intralesional, or perilesional routes, to exert local as well as systemic therapeutic effects. In certain embodiments, the compounds are administered to a mammal, in certain embodiments, a human, in a pharmaceutically acceptable dosage suitable for oral administration form such as those known in the art, and those discussed herein. For example, the compounds of this disclosure may be administered orally to a human as a liquid, or solid form. Solid dosage forms include, capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

The compounds provided herein may be useful for the treatment of any disease or condition described herein (e.g., any disease or condition wherein inhibition of USP28 is beneficial). In certain embodiments, the disease or condition is any disease or condition that benefits from the inhibition of USP28. In one embodiment, the disease or condition is a disorder of abnormal cellular proliferation mediated by USP28. Exemplary proliferative disorders include, but are not limited to, benign growths, neoplasms, tumors, cancer, autoimmune disorders, inflammatory disorders graft-versus-host rejection, and fibrotic disorders. Abnormal cellular proliferation, notably hyperproliferation, can occur as a result of a wide variety of factors, including genetic mutation, infection, exposure to toxins, autoimmune disorders, and benign or malignant tumor induction.

In one embodiment, a compound described herein is administered in an effective amount to a host, including a human, to treat a tumor, cancer (solid, non-solid, diffuse, hematological, etc.), abnormal cellular proliferation, immune disorder, inflammatory disorder, blood disorder, a myelo- or lymphoproliferative disorder such as B—or T-cell lymphomas, multiple myeloma, breast cancer, prostate cancer, AML, ALL, ACL, lung cancer, pancreatic cancer, colon cancer, skin cancer, melanoma, Waldenstrom's macroglobulinemia, Wiskott-Aldrich syndrome, or a post-transplant lymphoproliferative disorder; an autoimmune disorder, for example, Lupus, Crohn's Disease, Addison disease, Celiac disease, dermatomyositis, Graves disease, thyroiditis, multiple sclerosis, pernicious anemia, or reactive arthritis. In one embodiment, a compound described herein is administered in an effective amount to a host, including a human, to treat an infectious disease, including a viral and/or bacterial infection, an inflammatory condition, including asthma, chronic peptic ulcers, tuberculosis, rheumatoid arthritis, periodontitis, ulcerative colitis, or hepatitis.

In certain embodiments, the disease or condition is cancer. Non-limiting examples of cancer include breast cancer (e.g., invasive ductal breast cancer, noninvasive ductal breast cancer, inflammatory breast cancer), prostate cancer (e.g., hormone-dependent prostate cancer, hormone-independent prostate cancer), pancreatic cancer (e.g., ductal pancreatic cancer), gastric cancer (e.g., papillary adenocarcinoma, mucous adenocarcinoma, adenosquamous carcinoma), lung cancer (e.g., non-small cell lung cancer, small-cell lung cancer, malignant mesothelioma), colon cancer (e.g., gastrointestinal stromal tumor), rectal cancer (e.g., gastrointestinal stromal tumor), colorectal cancer (e.g., familial colorectal cancer, hereditary non-polyposis colorectal cancer, gastrointestinal stromal tumor), small intestinal cancer (e.g., non-Hodgkin's lymphoma, gastrointestinal stromal tumor), esophageal cancer, duodenal cancer, tongue cancer, pharyngeal cancer (e.g., nasopharyngeal cancer, oropharynx cancer, hypopharyngeal cancer), salivary gland cancer, brain tumor (e.g., pineal astrocytoma, pilocytic astrocytoma, diffuse astrocytoma, anaplastic astrocytoma), neurilemmoma, liver cancer (e.g., primary liver cancer, extrahepatic bile duct cancer), renal cancer (e.g., renal cell cancer, transitional cell cancer of the renal pelvis and ureter), bile duct cancer, endometrial cancer, uterine cervical cancer, ovarian cancer (e.g., epithelial ovarian cancer, extragonadal germ cell tumor, ovarian germ cell tumor, ovarian tumor of low malignant potential), bladder cancer, urethral cancer, skin cancer (e.g., intraocular (ocular) melanoma, Merkel cell carcinoma), hemangioma, malignant lymphoma, malignant melanoma, thyroid cancer (e.g., medullary thyroid cancer), parathyroid cancer, nasal cavity cancer, sinus cancer, bone tumor (e.g., osteosarcoma, Ewing tumor, uterine sarcoma, soft tissue sarcoma), angiofibroma, sarcoma of the retina, penis cancer, testicular tumor, pediatric solid tumor (e.g., Wilms' tumor, childhood kidney tumor), Kaposi's sarcoma, Kaposi's sarcoma caused by AIDS, tumor of maxillary sinus, fibrous histiocytoma, leiomyosarcoma, rhabdomyosarcoma, and leukemia (e.g., acute myeloid leukemia, acute lymphoblastic leukemia).

Additional non-limiting examples of cancer include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL)—also known as acute lymphoblastic leukemia or acute lymphoid leukemia (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In some embodiments, the cancer is a cancer that is sensitive to USP28 inhibition.

The cancer can be any cancer in any organ, for example, a cancer selected from the group consisting of glioma, thyroid carcinoma, breast carcinoma, small-cell lung carcinoma, non-small-cell carcinoma, gastric carcinoma, colon carcinoma, gastrointestinal stromal carcinoma, pancreatic carcinoma, bile duct carcinoma, CNS carcinoma, ovarian carcinoma, endometrial carcinoma, prostate carcinoma, renal carcinoma, anaplastic large-cell lymphoma, leukemia, multiple myeloma, mesothelioma, and melanoma, and combinations thereof.

In certain embodiments, the cancer is a solid tumor. A solid tumor, as used herein, refers to an abnormal mass of tissue that usually does not contain cysts or liquid areas. Different types of solid tumors are named for the type of cells that form them. Examples of classes of solid tumors include, but are not limited to, sarcomas, carcinomas, and lymphomas, as described above herein. Additional examples of solid tumors include, but are not limited to, squamous cell carcinoma, colon cancer, breast cancer, prostate cancer, lung cancer, liver cancer, pancreatic cancer, and melanoma.

In some embodiments, the cancer is characterized by gene amplification and/or elevated tumor expression of USP28, USP25, MYC, LSD1, NICD1, c-JUN, Notch-1, Claspin, CHK2, 53BP1, MDC1 and/or HIF-1α and/or reduced expression of FBXW7 relative to tissue-matched expression.

In some embodiments, the disease, or condition is an autoimmune disorder. Non-limiting examples of autoimmune disorders include, multiple sclerosis, experimental autoimmune encephalomyelitis, autoimmune disorder associated with immune rejection, graft versus host disease, uveitis, optic neuropathies, optic neuritis, transverse myelitis, inflammatory bowel disease, rheumatoid arthritis, ankylosing spondylitis, systemic lupus erythematosus, myasthenia gravis, and Graves disease.

Additional autoimmune disorders include intestine inflammatory disease, ulcerative colitis, Crohn's disease, and polyarthritis, local and systemic scleroderma, discoid lupus erythematosus, cutaneous lupus, cutaneous lupus erythematosus including chilblain lupus erythematosus, lupus nephritis, discoid lupus, subacute cutaneous lupus erythematosus, dermatomyositis, polymyositis, idiopathic myxedema, Hashimoto's disease, Guillain-Barre' syndrome, Grave's disease, Sjogren's syndrome, nodular panarteritis, autoimmune enteropathy, autoimmune oophoritis, chronic immune thrombocytopenic purpura, colitis, diabetes, pemphigus vulgaris, proliferative glomerulonephritis, Wiskott-Aldrich syndrome, autoimmune lymphoproliferative syndrome, chronic arthritis, inflammatory chronic rhinosinusitis, colitis, celiac disease, Barrett's esophagus, inflammatory gastritis, autoimmune nephritis, autoimmune vasculitis, autoimmune hepatitis, autoimmune carditis, autoimmune encephalitis, and autoimmune mediated hematological disease.

In one embodiment, the autoimmune disorder is psoriasis, a benign disease of human skin generally characterized by plaques covered by thickened scales. The disease is caused by increased proliferation of epidermal cells of unknown cause. Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma. Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue cause, for example, restenosis, retinopathies and atherosclerosis. Both cell migration and cell proliferation play a role in the formation of atherosclerotic lesions.

In some embodiments, the disease, or condition is an inflammatory disorder. Non-limiting examples of inflammatory disorders include, chronic rheumatoid arthritis, spondylitis deformans, arthritis deformans, lumbago, gout, post-operational or post-traumatic inflammation, bloating, neuralgia, laryngopharyngitis, cystitis, pneumonia, pancreatitis, enteritis, inflammatory bowel disease (including inflammatory large bowel disease), inflammation in metabolically important tissues including liver, fat, pancreas, kidney, and gut, and a proinflammatory state (e.g., elevated levels of proinflammatory cytokines or, markers of inflammation-like C-reactive protein in the blood).

In some embodiments, the disease or condition is a neurological disorder (e.g., neurodegenerative disorder), or a psychiatric disorder. Non-limiting examples of neurological disorders include, brain insulin resistance, mild cognitive impairment (MCI), Alzheimer's disease (AD), Parkinson's disease (PD), anxiety, dementia (e.g., senile dementia), traumatic brain injury, Huntington's chores, tardive dyskinesia, hyperkinesia, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis (ALS), glaucoma, and apoptosis-mediated degenerative diseases of the central nervous system (e.g., Creutzfeld-Jakob Disease, bovine spongiform encephalopathy (mad cow disease), chronic wasting syndrome).

In certain embodiments, the disease or condition is a viral infection, including, but not limited to, a virus selected from AIDS/HIV (Acquired Immune Deficiency Syndrome), amebiasis, avian Influenza (Bird flu), babesiosis, bird flu (Avian influenza), botulism, brucellosis, campylobacter infection, chancroid, chickenpox (varicella), chlamydia infections, ciguatera fish poisoning, coccidioidomycosis, colorado Tick Fever, (2019 Novel Coronavirus) (COVID-19), cryptosporidiosis, cysticercosis, dengue Fever, diphtheria, domoic acid poisoning (amnesic shellfish poisoning), E. coli Infections, Ebola Virus (viral hemorrhagic fever, ehrlichiosis, flu (influenza), gastroenteritis, viral German measles (rubella), giardia Infection, glanders, gonococcal Infection (gonorrhea), Haemophilus influenzae serotype B disease (Hib), hand-foot-and-mouth disease, hantavirus infections, hepatitis A, hepatitis B, hepatitis C, human immunodeficiency virus (HIV/AIDS), influenza (flu), Lassa Fever, legionellosis (Legionnaire's disease), leprosy (Hansen's disease), leptospirosis, listeriosis, lymphogranuloma venereum (LGV), malaria, Marburg virus hemorrhagic fever, melioidosis, measles, meningitis, meningococcal disease, Middle East Respiratory Syndrome Coronavirus (MERS-CoV), mumps, norovirus infection (Norwalk and Norwalk-like virus infection), non-gonococcal urethritis, paralytic shellfish poisoning, pertussis (whooping cough), pneumococcal disease, polio, psittacosis, rabies, relapsing Fever, Rocky Mountain Spotted Fever, respiratory syncytial virus (RSV), salmonellosis, scombroid fish poisoning, shigellosis, smallpox, syphilis, tetanus, toxoplasmosis, trichinosis (trichinellosis), tuberculosis (TB), tularemia, typhoid fever, typhus, varicella (chickenpox), viral gastroenteritis and norovirus, West Nile Virus, yellow Fever, yersiniosis (Yersinia enterocolitica), and Zika Virus.

In certain embodiments, provided herein are methods for the treatment that include, the administration of an effective amount of compounds provided herein, or a pharmaceutically acceptable salt thereof. In certain embodiments, the methods encompass the step of administering to the subject in need thereof an amount of a compound described herein effective for the treatment of disease, or condition in combination with a second agent effective for the treatment, or prevention of the disease, or condition. In certain embodiments, the compound is in the form of a pharmaceutical composition, or dosage form, as described elsewhere herein.

In certain embodiments, the subject is a treatment naïve subject. In further embodiments, the subject has previously received therapy. For instance, in certain embodiments, the subject has not responded to a single agent treatment regimen.

In certain embodiments, the subject is a subject that discontinued some other therapy because of one or more adverse events associated with the other therapy. In certain embodiments, the subject has received some other therapy and discontinued that therapy prior to administration of a method provided herein. In further embodiments, the subject has received therapy and continues to receive that therapy along with administration of a compound provided herein. The compounds described herein can be co-administered with other therapy for treatment of the disease or condition according to the judgment of one of skill in the art. In certain embodiments, the methods or compositions provided herein can be co-administered with a reduced dose of the other therapy for the treatment of the disease or condition.

### Diagnostic Applications

In some embodiments, the compounds provided herein are used in diagnostic applications. These applications may be useful, for example, in making a diagnosis, and/or prognosis for a disease, or condition, such as a metabolic disease, or condition.

In some diagnostic and prognostic applications or embodiments, the compound may be labeled with a detectable moiety. Suitable detectable moieties include, but are not limited to, radioisotopes, fluorescent labels, and enzyme-substrate labels. In another embodiment, the compound need not be labeled, and the presence of the compound can be detected using a labeled antibody, or antigen binding fragment thereof which specifically binds to the compound.

### Kits

In some embodiments, a compound provided herein is provided in the form of a kit (i.e., a packaged combination of reagents in predetermined amounts with instructions for performing a procedure). In some embodiments, the procedure is a diagnostic assay. In certain embodiments, the procedure is a therapeutic procedure.

In some embodiments, the kit further comprises a solvent for the reconstitution of the compound. In some embodiments, the compound is provided in the form of a pharmaceutical composition.

In some embodiments, the kits can include a compound, or composition provided herein, an optional second agent, or composition, and instructions providing information to a health care provider regarding usage for treating the disorder. Instructions may be provided in printed form, or in the form of an electronic medium such as a floppy disc, CD, or DVD, or in the form of a website address where such instructions may be obtained. A unit dose of a compound, or a composition provided herein, or a second agent, or composition, can include a dosage such that when administered to a subject, a therapeutically, or prophylactically effective plasma level of the compound, or composition can be maintained in the subject for at least one day. In some embodiments, a compound, or composition can be included as a sterile aqueous pharmaceutical composition, or dry powder (e.g., lyophilized) composition.

In some embodiments, suitable packaging is provided. As used herein, “packaging” includes a solid matrix, or material customarily used in a system, and capable of holding within fixed limits a compound provided herein, and/or a second agent suitable for administration to a subject. Such materials include, glass, and plastic (e.g., polyethylene, polypropylene, and polycarbonate) bottles, vials, paper, plastic, plastic-foil laminated envelopes, and the like. If e-beam sterilization techniques are employed, the packaging should have sufficiently low density to permit sterilization of the contents.

### Preparation and Synthetic Procedures

In some embodiments, the compounds described herein are prepared as outlined in Schemes 1-5. The synthesis of the compounds in this application is not limited to these general reaction schemes illustrated here. For detailed synthesis of each individual compound, please check the Examples section.

### Examples

### Preparation of the Compounds

The compounds used in the reactions described herein are made according to organic synthesis techniques known to those skilled in this art, starting from commercially available chemicals, and/or from compounds described in the chemical literature. “Commercially available chemicals” are obtained from standard commercial sources such as Acros Organics (Pittsburgh, PA), Advanced ChemBlocks, Inc (Burlingame, CA), Aldrich Chemical (Milwaukee, WI, including Sigma Chemical and Fluka), AK Scientific (Union City, CA), AstaTech, Inc. (Bristol, PA), Aurum Pharmatech LLC (Franklin Park, NJ), Combi-Blocks, Inc. (San Diego, CA), Enamine (Monmouth Jct., NJ), Fisher Scientific Co. (Pittsburgh, PA), Frontier Scientific (Logan, UT), TCI America (Portland, OR), and VWR (Radnor, PA). Specific and analogous reactants are optionally identified through the indices of known chemicals prepared by the Chemical Abstract Service of the American Chemical Society, which are available in most public and university libraries, as well as through on-line databases.

Suitable reference books that detail the synthesis of reactants useful in the preparation of compounds described herein, or provide references to articles that describe their preparation, include for example, “Synthetic Organic Chemistry”, John Wiley & Sons, Inc., New York; S. R. Sandler et al., “Organic Functional Group Preparations,” 2nd Ed., Academic Press, New York, 1983; “T. L. Gilchrist, “Heterocyclic Chemistry”, 2nd Ed., John Wiley & Sons, New York, 1992; and J. March, “Advanced Organic Chemistry: Reactions, Mechanisms and Structure”, 4th Ed., Wiley-Interscience, New York, 1992; R. C. Larock “Comprehensive Organic Transformations: A Guide to Functional Group Preparations” 2nd Edition (1999) Wiley-VCH, ISBN: 0-471-19031-4; “Organic Reactions” (1942-2000) John Wiley & Sons, in over 55 volumes; and “Chemistry of Functional Groups” John Wiley & Sons, in 73 volumes. Some compounds require the application of protecting groups. The need for such protection is within the skill in the art. For a general description of protecting groups and their use, see T. W. Greene, P. G. M. Nuts, and Protective Groups in Organic Synthesis, John Wiley & Sons, New York, 1999.

### Analytical Methods and Instrumentation

Proton nuclear magnetic resonance (NMR) spectra were obtained on either Bruker, or Varian spectrometers at 400, or 600 MHz. NMR spectra are reported relative to residual solvent signals as follows: chemical shift δ (ppm), multiplicity, coupling constant J(Hz), and integration. Tetramethylsilane (TMS) was used as an internal standard in some of the cases. Mass spectral data were measured using one of the two following systems: System A: Waters Acquity i-class ultra-performance liquid chromatography (UPLC) system with Acquity Photo Diode Array Detector, Acquity Evaporative Light Scattering Detector (ELSD) and Waters ZQ Mass Spectrometer. Data was acquired using Waters MassLynx 4.1 software and purity characterized by UV wavelength 220 nm, evaporative light scattering detection (ELSD) and electrospray positive ion (ESI) (column: Acquity UPLC BEH C18 1.7μι2.1×50 mm). System B: Agilent LC/MS consisting of a 1200 series LC and 6140 Quadrupole MS detector (column: Agilent USGYL01131, HPH-C18 2.7 μM, 2.1×50 mm). Solvents used: acetonitrile/water, containing 0.1 formic acid; flow rate 0.7 mL/min. Preparatory HPLC purifications were conducted with a flow rate of 15 mL/min and detection by UV wavelength 214 nm and 254 nm (Column: Jupiter© 10 M Proteo 90 Å, 250×21.2 mm A, solvent: acetonitrile/water, containing modifier such as 0.1% trifluoroacetic acid, formic acid or acetic acid). Compound purity was checked on an analytical HPLC (Waters Acquity UPLC H-Class instrument), with a flow rate of 0.5 mL/min (Acquity BEH C18, 50×2.1 mm column).

### Abbreviations

| Abbreviation | Name |
|---|---|
| CH₃CN | acetonitrile |
| aq. | aqueous |
| atm | atmospheres |
| BINAP | (1,1′-binaphthalene-2,2′-diyl)bis(diphenylphosphine) |
| Boc | t-butoxycarbonyl |
| CCl₄ | carbon tetrachloride |
| CDCl₃ | deuterated chloroform |
| CO | carbon monoxide gas |
| CO₂ | carbon dioxide |
| Cs₂CO₃ | cesium carbonate |
| CuBr | copper(I) bromide |
| Cu(OAc)₂ | copper(II) acetate |
| DCC | dicyclohexylcarbodiimide |
| DCM | dichloromethane |
| DEAD | diethyl azodicarboxylate |
| DIPEA | diisopropylethylamine |
| DMF | N,N-dimethylformamide |
| DMSO | dimethylsulfoxide |
| ESI | electrospray ionization |
| Et₃N | triethylamine |
| EtOAc | ethyl acetate |
| EtOH | ethanol |
| h | hours |
| H₂O | water |
| HATU | 1-[bis(dimethylamino)methylene]-1H-1,2,3-triazolo[4,5- |
| | b]pyridinium 3-oxid hexafluorophosphate |
| HCl | hydrochloric acid |
| HOAc | acetic acid |
| K₂CO₃ | potassium carbonate |
| KI | potassium iodide |
| KOH | potassium hydroxide |
| KOtBu | potassium tert-butoxide |
| LiAlH₄ | lithium aluminum hydride |
| LiOH | lithium hydroxide |
| MeOH | methanol |
| min | minutes |
| N₂ | nitrogen |
| Na₂CO₃ | sodium carbonate |
| Na₂SO₄ | sodium sulfate |
| NaH | sodium hydride |
| NaHCO₃ | sodium bicarbonate |
| NaN₃ | sodium azide |
| NaOH | sodium hydroxide |
| NBS | N-bromosuccinimide |
| NH₄Cl | ammonium chloride |
| NH₄HCO₃ | ammonium bicarbonate |
| NMR | nuclear magnetic resonance |
| Pd₂(dba)₃ | tris(dibenzylideneacetone)dipalladium(0) |
| Pd(dppf)Cl₂ | [1,1′-bis(diphenylphosphino)ferrocene]dichloropalladium(II) |
| Pd/C | palladium on carbon |
| Pd(OAc)₂ | palladium(II) acetate |
| Prep-TLC | preparatory thin layer chromatography |
| RuPhos-Pd- | chloro(2-dicyclohexylphosphino-2′,6′-diisopropoxy-1,1′- |
| G2 | biphenyl)[2-(2′-amino-1,1′-biphenyl)]palladium(II) |
| sat. | saturated |
| t-BuOH | tert-butanol |
| T₃P | propylphosphonic anhydride |
| TBAI | tetra-n-butylammonium iodide |
| TBD | 1,5,7-triazabicyclo[4.4.0]dec-5-ene |
| TFA | trifluoracetic acid |
| THF | tetrahydrofuran |

### General Schemes

Unless otherwise noted, reagents, and solvents were used as received from commercial suppliers. Anhydrous solvents and oven-dried glassware were used for synthetic transformations sensitive to moisture, and/or oxygen. Reaction times, and yields were not optimized. Example numbers and compound numbers are the same.

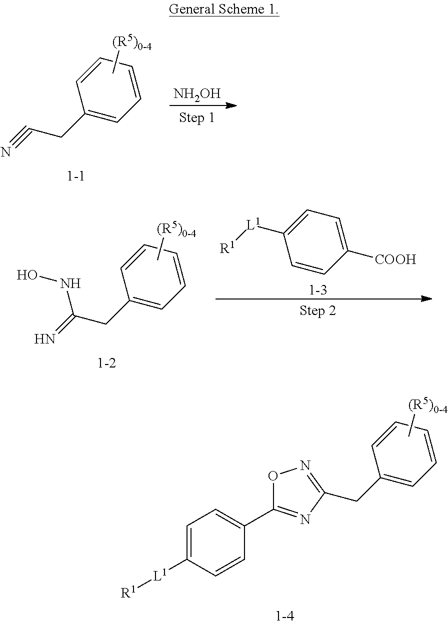

In Step 1, 2-phenylacetonitrile intermediate 1-1 substituted with 0-4 R⁵ groups is converted to its corresponding N-hydroxy-2-phenylacetimidamide intermediate 1-2 using hydroxylamine. In Step 2, the N-hydroxy-2-phenylacetimidamide intermediate 1-2 reacts with carboxylic acid-containing compound 1-3 to afford the 1,2,4-oxadiazole compound 1-4 of Formula (I).

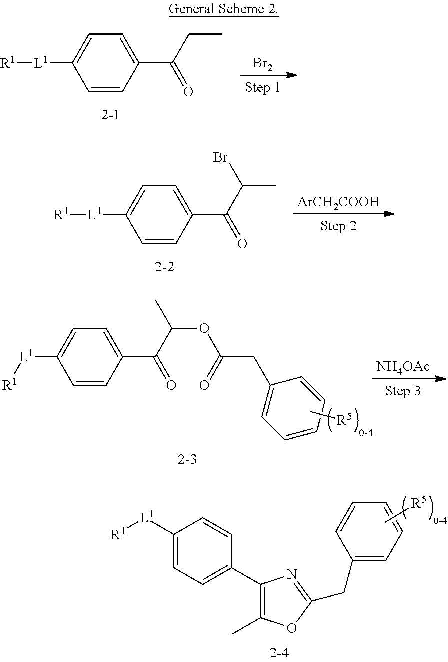

In Step 1, ketone-containing intermediate 2-1 is treated with Br₂ in the presence of acid to form intermediate 2-2, which is reacted with 2-phenylacetic acid to form intermediate 2-3. In step 3, intermediate 2-3 is then reacted with ammonium acetate to form oxazole compound 2-4 of Formula (II).

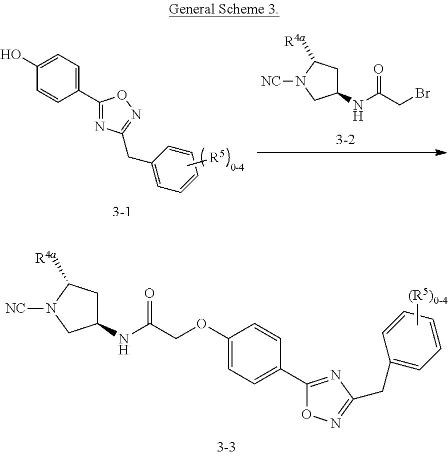

1,2,4-Oxadiazole intermediate 3-1 is reacted with bromoacetamide intermediate 3-2 under S_{N}2 reaction conditions to form 1,2,4-oxadiazole compound 3-3 of Formula (I).

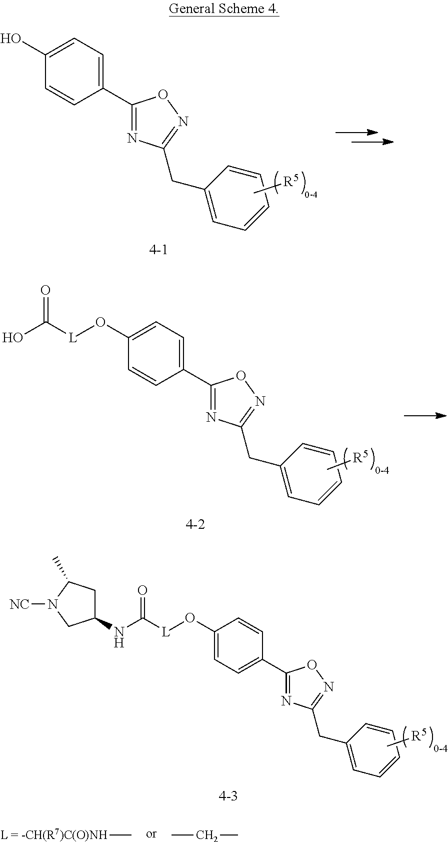

First, 1,2,4-oxadiazole intermediate 4-1 is subjected to appropriate conditions to install a linker that terminates in a carboxylic acid. In the final step, intermediate 4-2 undergoes a condensation reaction with (2R,4R)-4-amino-2-methylpyrrolidine-1-carbonitrile to afford compound 4-3 of Formula (I).

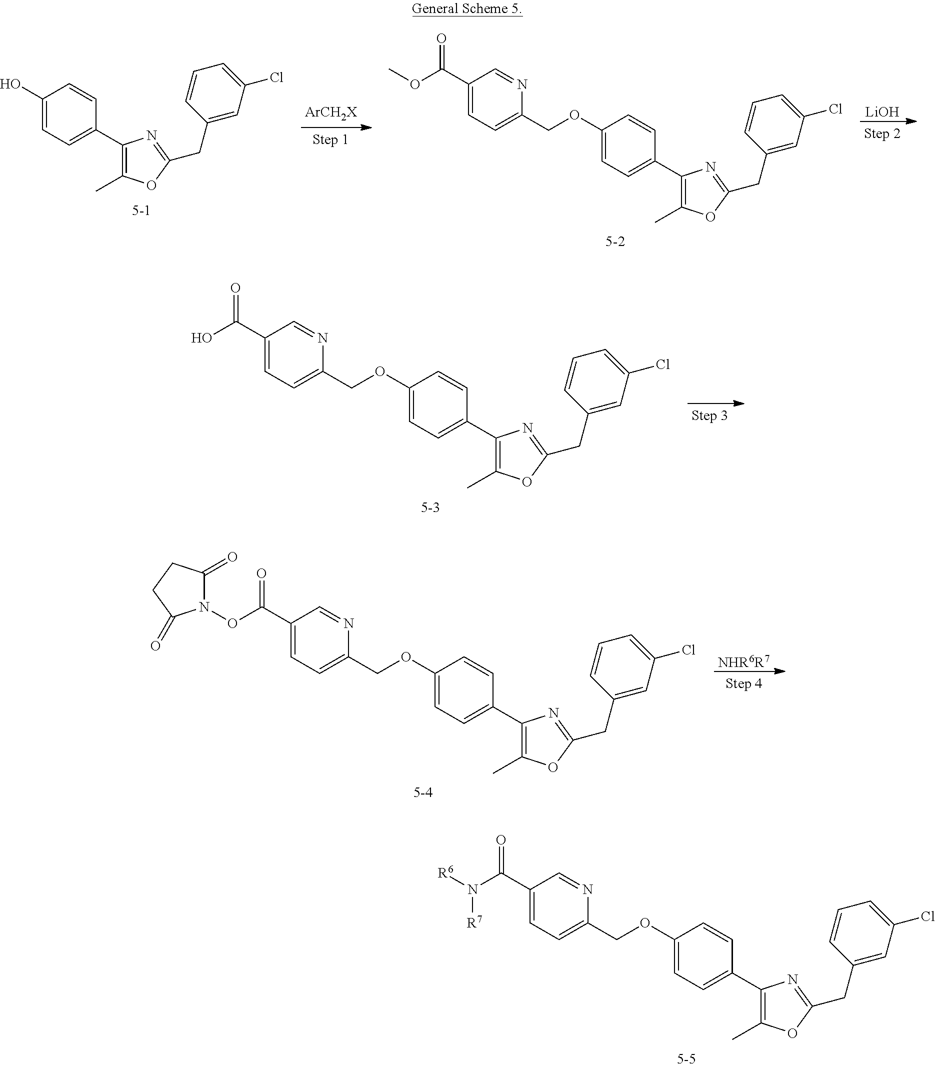

In step 1, intermediate 5-1 is reacted with a halogen substituted benzyl compound to afford intermediate 5-2, which is then subjected to hydrolysis conditions to afford intermediate 5-3. In step 3, intermediate 5-3 is reacted with succinimide to afford intermediate 5-4, which is reacted with an appropriate amine to afford compound 5-5 of Formula (II).

### Preparation of Representative Compounds

### Example 1. 2-(4-(3-(3-Chlorobenzyl)-1,2,4-oxadiazol-5-yl)phenoxy)-N-((3R,5R)-1-cyano-5-methylpyrrolidin-3-yl)acetamide (Compound 3)

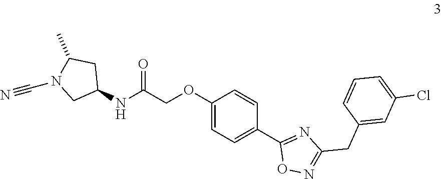 [SMILES: C[C@@H]1C[C@@H](NC(=O)COc2ccc(-c3nc(Cc4cccc(Cl)c4)no3)cc2)CN1C#N]

### Step A. 2-(3-Chlorophenyl)-N-hydroxyacetimidamide (3a)

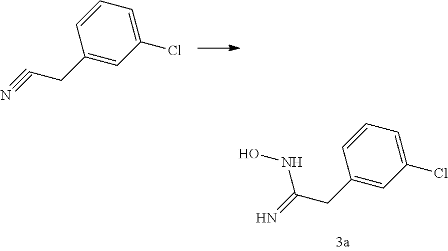 [SMILES: N#CCc1cccc(Cl)c1 ; N=C(Cc1cccc(Cl)c1)NO]

To a solution of 2-(3-chlorophenyl) acetonitrile (0.5 g, 3.3 mmol) in EtOH (20 mL) was added K₂CO₃ (685 mg, 4.9 mmol) and NH₂OH·HCl (343 mg, 4.9 mmol). The reaction was stirred at 80° C. for 18 h. After completion, the reaction was filtered and concentrated to afford the crude title product (3a) (0.4 g) as a white solid, which was used in the next step without further purification. m/z (ESI, +ve ion)=185.1 [M+H]⁺.

### Step B. 4-(3-(3-Chlorobenzyl)-1,2,4-oxadiazol-5-yl)phenol (3b)

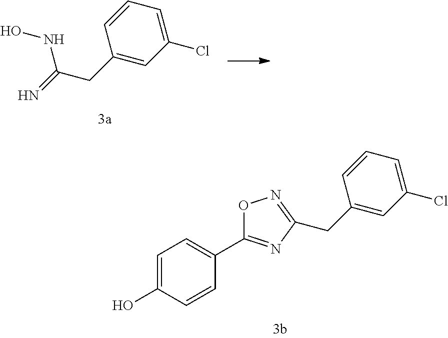 [SMILES: N=C(Cc1cccc(Cl)c1)NO ; Oc1ccc(-c2nc(Cc3cccc(Cl)c3)no2)cc1]

To a solution of 4-hydroxybenzoic acid (0.4 g, 2.9 mmol) in NMP (20 mL) was added CDI (563 mg, 3.4 mmol). After stirring at 50° C. for 1 h, 3a (515 mg, 2.8 mmol) was added and the reaction was stirred at 120° C. for 18 h. After completion, the mixture was quenched with water (20 mL) and extracted with EtOAc (50 mL×2). The combined organic layer was dried over Na₂SO₄, filtered, and concentrated. The crude residue was purified by silica gel column chromatography (petroleum ether:EtOAc=1:1) to afford the title product (3b) (106.1 mg, 13%) as a white solid. m/z (ESI, +ve ion)=287.1 [M+H]⁺.

### Step C. 2-Bromo-N-((3R,5R)-1-cyano-5-methylpyrrolidin-3-yl)acetamide (3c)

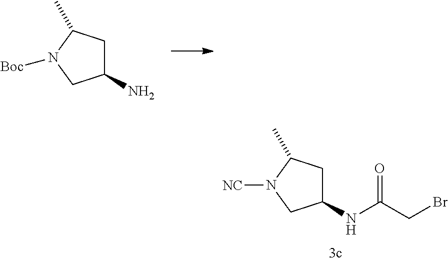

To a solution of tert-butyl (2R,4R)-4-amino-2-methylpyrrolidine-1-carboxylate (40 mg, 0.20 mmol) in DCM (2 mL) at 0° C. was added 2,2,6,6-tetramethylpiperidine (33 μL, 0.20 mmol) and bromoacetyl bromide (19 μL, 0.22 mmol). After stirring for 30 min at 0° C., 5 mL water was added. The aqueous layer was separated from the reaction. To the organic layer was added TFA (1 mL), and the layer was stirred for 30 min and concentrated. The residue was redissolved in DCM (2 mL). DIPEA (0.1 mL, 0.57 mmol) was added, followed by addition of cyanogen bromide (25 mg, 0.24 mmol). The mixture was stirred 0° C. for 1 h and concentrated. The crude residue was purified by silica gel column chromatography (gradient elution, 50-80% EtOAc in hexanes) to provide the title product (3c) (33 mg, 67%) as a colorless oil. m/z (ESI, +ve ion)=248.1 [M+2+H]⁺.

### Step D. 2-(4-(3-(3-Chlorobenzyl)-1,2,4-oxadiazol-5-yl)phenoxy)-N-((3R,5R)-1-cyano-5-methylpyrrolidin-3-yl)acetamide (3)

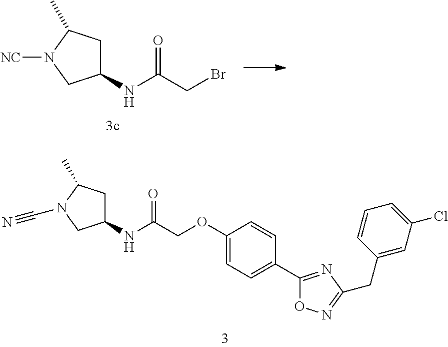 [SMILES: C[C@@H]1C[C@@H](NC(=O)CBr)CN1C#N ; C[C@@H]1C[C@@H](NC(=O)COc2ccc(-c3nc(Cc4cccc(Cl)c4)no3)cc2)CN1C#N]

To a solution of 3b (3.4 mg, 0.012 mmol) in DMF (0.3 mL) was added K₂CO₃ (3.3 mg, 0.024 mmol), followed by a 0.1 M solution of 3c in DMF (0.12 mL, 0.012 mmol). The reaction was stirred at room temperature for 1.5 h and purified directly by reverse phase HPLC (CH₃CN in water, with 0.1% HOAc as a modifier) to provide the title product (3) (4.2 mg, 77%) as a white solid. m/z (ESI, +ve ion)=452.2 [M+H]⁺.

Compounds 1, 2, 4, 6, 7, 9, and 12 were synthesized in similar procedures as described for the synthesis of Compound 3 in Example 1.

### Example 2. (E)-3-(4-(3-(3-Chlorobenzyl)-1,2,4-oxadiazol-5-yl)phenyl)-N-((3R,5R)-1-cyano-5-methylpyrrolidin-3-yl)acrylamide (Compound 5)

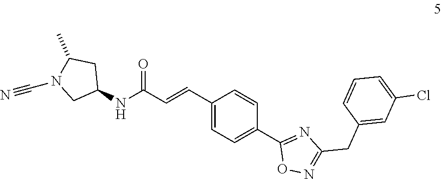 [SMILES: C[C@@H]1C[C@@H](NC(=O)/C=C/c2ccc(-c3nc(Cc4cccc(Cl)c4)no3)cc2)CN1C#N]

### Step A. 3-(3-Chlorobenzyl)-5-(4-iodophenyl)-1,2,4-oxadiazole (5a)

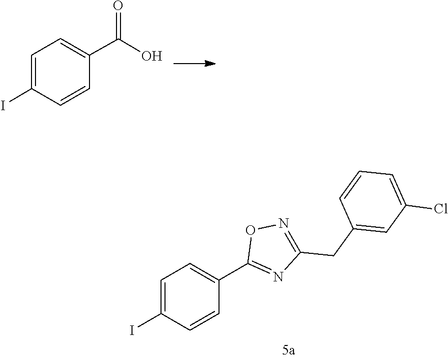 [SMILES: O=C(O)c1ccc(I)cc1 ; Clc1cccc(Cc2noc(-c3ccc(I)cc3)n2)c1]

To a solution of 4-iodobenzoic acid (50 mg, 0.2 mmol) in NMP (2 mL) was added CDI (33 mg, 0.24 mmol). After stirring at 50° C. for 30 min, 3a (37 mg, 0.20 mmol) was added, and the reaction was heated to 120° C. for 18 h. After completion, water was added (10 mL), and the reaction was extracted with EtOAc (20 mL). The organic layer was washed with H₂O (10 mL), dried over Na₂SO₄, filtered, concentrated and purified by silica gel column chromatography (petroleum:EtOAc=1:2) to provide the title product (5a) (20 mg, 18.6%) as a yellow solid. m/z (ESI, +ve ion)=397.1 [M+H]⁺.

### Step B. Ethyl (E)-3-(4-(3-(3-chlorobenzyl)-1,2,4-oxadiazol-5-yl)phenyl)acrylate (5b)

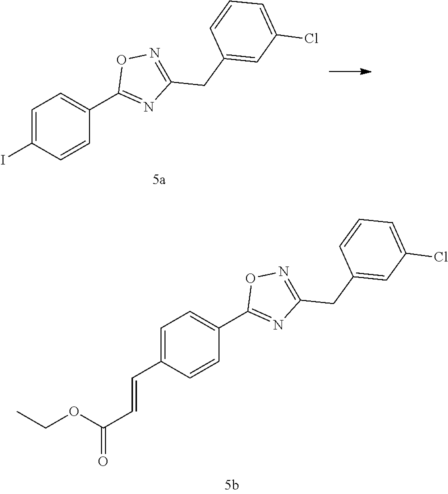 [SMILES: Clc1cccc(Cc2noc(-c3ccc(I)cc3)n2)c1 ; CCOC(=O)/C=C/c1ccc(-c2nc(Cc3cccc(Cl)c3)no2)cc1]

To the stirred solution of 5a (250 mg, 0.63 mmol) in DMF (10 mL) was added ethyl acrylate (63 mg, 0.63 mmol), Pd(OAc)₂ (25 mg, 0.063 mmol), PPh₃ (16.5 mg, 0.063 mmol), and Et₃N (190 mg, 1.89 mmol) under N₂. The reaction was heated to 100° C. and stirred for 14 h. After completion, the resulting mixture was concentrated and purified by silica gel column chromatography (EtOAc:petroleum ether=10:90) to provide the title product (5b) (230 mg, 95%) as a yellow oil. m/z (ESI, +ve ion)=369.0 [M+H]⁺.

### Step C. (E)-3-(4-(3-(3-Chlorobenzyl)-1,2,4-oxadiazol-5-yl)phenyl)acrylic acid (5c)

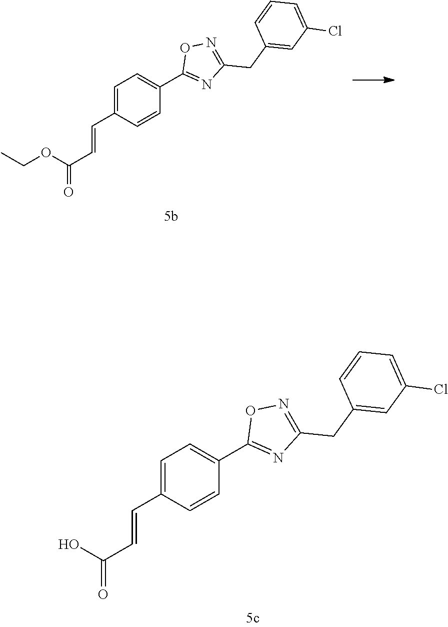 [SMILES: CCOC(=O)/C=C/c1ccc(-c2nc(Cc3cccc(Cl)c3)no2)cc1 ; O=C(O)/C=C/c1ccc(-c2nc(Cc3cccc(Cl)c3)no2)cc1]

To a solution of 5b (200 mg, 0.54 mmol) in THF (4 mL) and water (4 mL) was added NaOH (55 mg, 2.75 mmol). The reaction mixture was stirred at 25° C. for 2 h, then diluted with EtOAc (20 mL) and ice-water (20 mL). The organic layer was washed with water (10 mL×3), brine (10 mL), dried over anhydrous sodium sulfate, filtered, concentrated and purified by prep-TLC (DCM:MeOH=20:1) to afford the title product (5c) (111.4 mg, 60%) as a white solid. m/z (ESI, −ve ion)=339.0 [M−H]⁻.

### Step D. (2R,4R)-4-Amino-2-methylpyrrolidine-1-carbonitrile (5d)

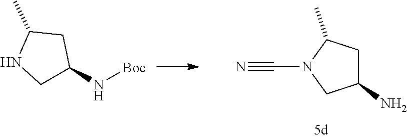

To a stirred solution of tert-butyl ((3R,5R)-5-methylpyrrolidin-3-yl)carbamate (100 mg, 0.050 mmol), DIPEA (0.13 mL, 0.75 mmol) and dichloromethane (1.0 mL) was added cyanogen bromide (58 mg, 0.055 mmol) in dichloromethane (0.5 mL) at 0° C. After stirring for 25 min at room temperature, the mixture was quenched with water (2.0 mL). The organic layer was separated and the aqueous layer was extracted with dichloromethane (2 mL). The combined organic layer was dried over MgSO₄, concentrated and redissolved in HCl (3.0 mL, 4 N in 1,4-dioxane). The reaction was stirred at room temperature for 1 h. Solid precipitated out. The supernatant was decanted. To the remaining solid was added 1,4-dioxane (3 mL) and the reaction vial was sonicated and then concentrated. The same procedure was repeated with CH₃CN (2 mL)). The resulting solid was lyophilized to provide the crude title product (5d) (HCl salt), which was used in the next step without further purification. m/z (ESI, +ve ion)=126.3 [M+H]⁺.

### Step E. (E)-3-(4-(3-(3-Chlorobenzyl)-1,2,4-oxadiazol-5-yl)phenyl)-N-((3R,5R)-1-cyano-5-methylpyrrolidin-3-yl)acrylamide (5)

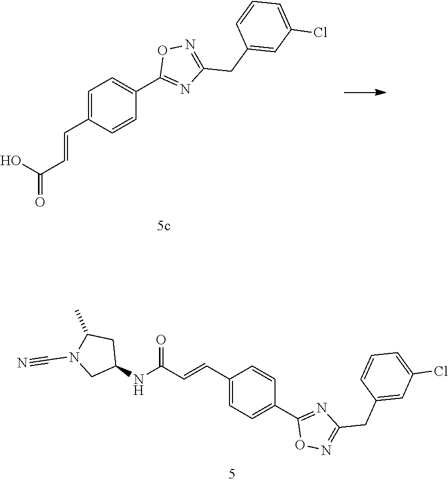 [SMILES: O=C(O)/C=C/c1ccc(-c2nc(Cc3cccc(Cl)c3)no2)cc1 ; C[C@@H]1C[C@@H](NC(=O)/C=C/c2ccc(-c3nc(Cc4cccc(Cl)c4)no3)cc2)CN1C#N]

A solution of 5c (4.1 mg, 0.012 mmol), 5d (3 mg, crude), HATU (6.8 mg, 0.018 mmol), and DIPEA (0.0063 mL, 0.036 mmol) in DMF (0.3 mL) was stirred at room temperature for 3 h. The mixture was directly purified by reverse phase HPLC (CH₃CN in water, with 0.1% HOAc as a modifier) to provide the title product (5) (4.7 mg, 87%) as a white solid. m/z (ESI, +ve ion)=448.2 [M+H]⁺. ¹H NMR (400 MHz, DMSO-d₆) δ ppm 8.61 (d, J=6.60 Hz, 1H), 8.12 (d, J=8.31 Hz, 2H), 7.80 (d, J=8.56 Hz, 2H), 7.28-7.59 (m, 5H), 6.77 (d, J=15.89 Hz, 1H), 4.38 (qt, J=5.97, 2.90 Hz, 1H), 4.23 (s, 2H), 3.66-3.92 (m, 2H), 3.26 (dd, J=9.90, 2.57 Hz, 1H), 2.01 (ddd, J=12.90, 6.17, 2.93 Hz, 1H), 1.74 (ddd, J=12.96, 8.80, 6.11 Hz, 1H), 1.26 (d, J=6.11 Hz, 3H).

### Example 3. N-((3R,5R)-1-Cyano-5-methylpyrrolidin-3-yl)-2-(4-(3-(3-cyanobenzyl)-1,2,4-oxadiazol-5-yl)phenoxy)acetamide (Compound 8)

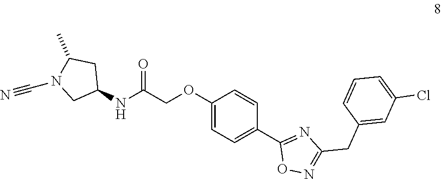 [SMILES: C[C@@H]1C[C@@H](NC(=O)COc2ccc(-c3nc(Cc4cccc(Cl)c4)no3)cc2)CN1C#N]

### Step A. 3-((5-(4-Hydroxyphenyl)-1,2,4-oxadiazol-3-yl)methyl)benzonitrile (8b)

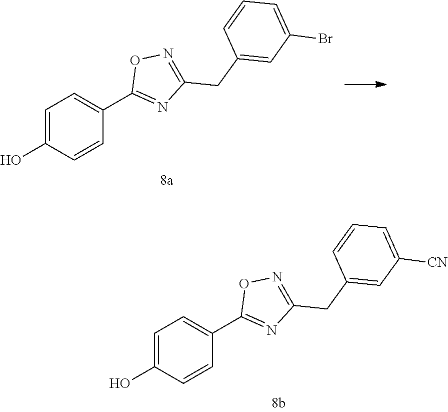 [SMILES: Oc1ccc(-c2nc(Cc3cccc(Br)c3)no2)cc1 ; N#Cc1cccc(Cc2noc(-c3ccc(O)cc3)n2)c1]

To a solution of 8a (prepared following similar procedures as 3b, 800 mg, 2.42 mmol) in NMP (15 mL) was added copper cyanide (649 mg, 7.25 mmol). The reaction was heated at 180° C. for 0.5 h. After cooling down, the reaction mixture was filtered through a pad of celite. The filtrate was diluted with EtOAc (50 mL) and washed with brine (50 mL×3). The organic layer was dried, filtered, concentrated and purified by silica gel column chromatography (petroleum ether:EtOAc=2:1) to afford the title product (8b) (75 mg, 10.7%) as a yellow solid. m/z (ESI, +ve ion)=278.0 [M+H]⁺.

### Step B. N-((3R,5R)-1-Cyano-5-methylpyrrolidin-3-yl)-2-(4-(3-(3-cyanobenzyl)-1,2,4-oxadiazol-5-yl)phenoxy)acetamide (8)

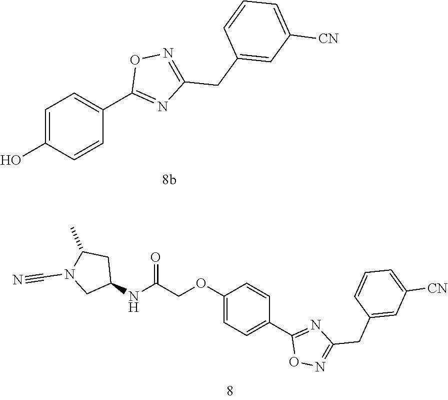 [SMILES: N#Cc1cccc(Cc2noc(-c3ccc(O)cc3)n2)c1 ; C[C@@H]1C[C@@H](NC(=O)COc2ccc(-c3nc(Cc4cccc(C#N)c4)no3)cc2)CN1C#N]

The title product (Compound 8) was synthesized in a similar procedure as described for the synthesis of Compound 3 in Example 1, Step D. m/z (ESI, +ve ion)=443.3 [M+H]⁺.

### Example 4. 1-(4-(3-(3-Chlorobenzyl)-1,2,4-oxadiazol-5-yl)benzyl)-3-((3R,5R)-1-cyano-5-methylpyrrolidin-3-yl)urea (Compound 10)

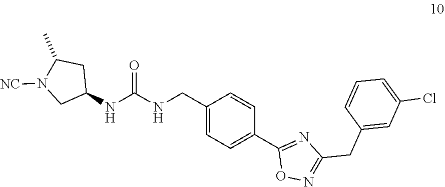 [SMILES: C[C@@H]1C[C@@H](NC(=O)NCc2ccc(-c3nc(Cc4cccc(Cl)c4)no3)cc2)CN1C#N]

### Step A. 4-(((tert-Butoxycarbonyl)amino)methyl)benzoic acid (10a)

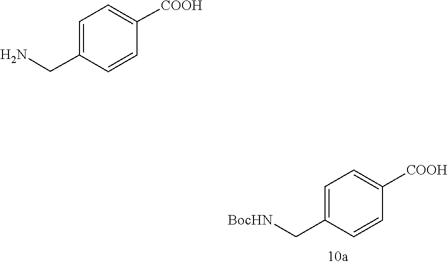 [SMILES: NCc1ccc(C(=O)O)cc1 ; CC(C)(C)OC(=O)NCc1ccc(C(=O)O)cc1]

To a solution of 4-(aminomethyl) benzoic acid (0.5 g, 3.31 mmol) in 1,4-dioxane and water (1:1, 6 mL) was added (BOC)₂O (0.938 g, 4.30 mmol) and NaOH (0.265 g, 6.62 mmol) at 0° C. The reaction mixture was stirred at 15° C. for 12 h. Then the solvent was evaporated in vacuo to provide the crude title product (10a), which was used in the next step without further purification. m/z (ESI, +ve ion)=196.1 [M+H]⁺.

### Step B. tert-Butyl (4-(3-(3-chlorobenzyl)-1,2,4-oxadiazol-5-yl)benzyl)carbamate (10b)

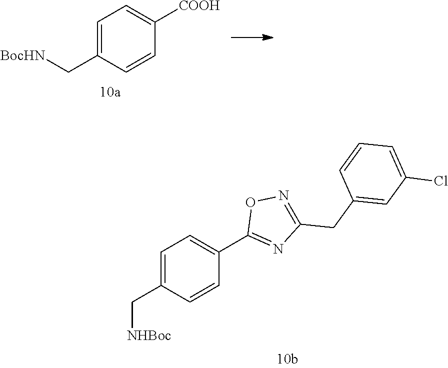 [SMILES: CC(C)(C)OC(=O)NCc1ccc(C(=O)O)cc1 ; CC(C)(C)OC(=O)NCc1ccc(-c2nc(Cc3cccc(Cl)c3)no2)cc1]

A flask was charged with 10a (300 mg, 1.19 mmol) and CDI (202.2 mg, 1.43 mmol). NMP (5 mL) was added, and the reaction was stirred at 50° C. for 0.5 h. Then 3a (329.55 mg, 1.785 mmol) was added and the reaction mixture was heated at 120° C. for 16 h. After cooling down, the reaction was quenched with water (10 mL) and extracted with DCM (30 mL). The organic layer was washed with water (30 mL), dried, filtered and concentrated to afford the crude title product (10b) (300 mg), which was used in the next step without further purification. m/z (ESI, +ve ion)=400.0 [M+H]⁺.

### Step C. (4-(3-(3-Chlorobenzyl)-1,2,4-oxadiazol-5-yl)phenyl)methanamine (10c)

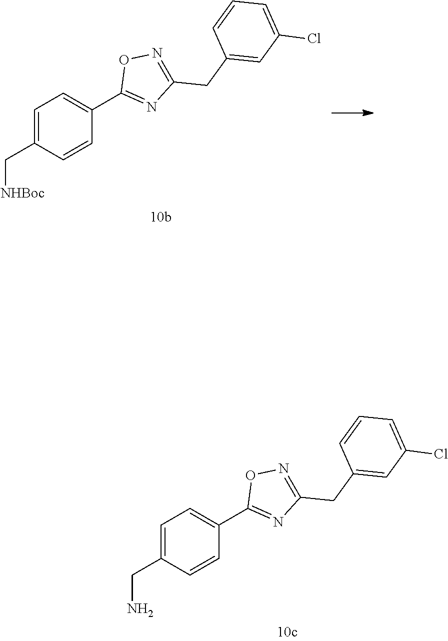 [SMILES: CC(C)(C)OC(=O)NCc1ccc(-c2nc(Cc3cccc(Cl)c3)no2)cc1 ; NCc1ccc(-c2nc(Cc3cccc(Cl)c3)no2)cc1]

TFA (2 mL) was added to a solution of 10b (300 mg, 0.75 mmol) in DCM. The reaction mixture was stirred at 15° C. for 2 h. Then the solvent was evaporated in vacuo and the crude residue was purified by silica gel column chromatography (DCM/MeOH=10:1) to afford the title product (10c) (85.5 mg, 37%) as a white solid. m/z (ESI, +ve ion)=300.1 [M+H]⁺.

### Step D. tert-Butyl (2R,4R)-4-(3-(4-(3-(3-chlorobenzyl)-1,2,4-oxadiazol-5-yl)benzyl)ureido)-2-methylpyrrolidine-1-carboxylate (10d)

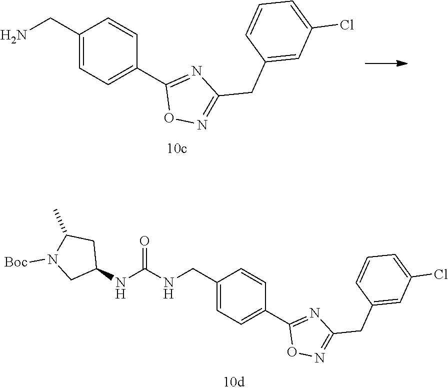 [SMILES: NCc1ccc(-c2nc(Cc3cccc(Cl)c3)no2)cc1 ; C[C@@H]1C[C@@H](NC(=O)NCc2ccc(-c3nc(Cc4cccc(Cl)c4)no3)cc2)CN1C(=O)OC(C)(C)C]

To a solution of tert-butyl (2,4R)-4-amino-2-methylpyrrolidine-1-carboxylate (6.0 mg, 0.030 mmol) in DMF (0.3 mL) was added DIPEA (0.01 mL, 0.060 mmol) and CDI (4.9 mg, 0.030 mmol). The reaction was stirred at room temperature for 20 min (solution A). To solution A was added 10c (6.0 mg, 0.020 mmol) and the resulting mixture was stirred overnight. However, the reaction showed incomplete conversion. Another solution A was prepared and added to the reaction. After stirring for 3 h, the reaction was purified by reverse phase HPLC (CH₃CN in water, with 0.1% HOAc as a modifier) to provide the title product (10d) (10.8 mg, quantitative) as a white solid. m/z (ESI, +ve ion)=426.2 [M-Boc+H]⁺.

### Step E. 1-(4-(3-(3-Chlorobenzyl)-1,2,4-oxadiazol-5-yl)benzyl)-3-((3R,5R)-1-cyano-5-methylpyrrolidin-3-yl)urea (10)

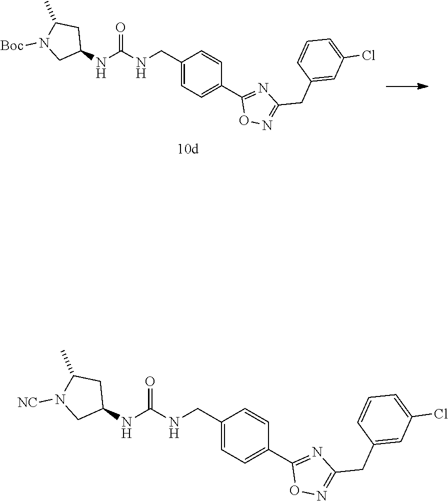 [SMILES: C[C@@H]1C[C@@H](NC(=O)NCc2ccc(-c3nc(Cc4cccc(Cl)c4)no3)cc2)CN1C(=O)OC(C)(C)C ; C[C@@H]1C[C@@H](NC(=O)NCc2ccc(-c3nc(Cc4cccc(Cl)c4)no3)cc2)CN1C#N]

To a solution of 10d (10.8 mg, 0.02 mmol) in DCM (0.4 mL) was added TFA (0.4 mL) and the reaction was stirred at room temperature for 40 min. The reaction was concentrated under reduced pressure and redissolved in DCM (0.3 mL). DIPEA (0.01 mL, 0.060 mmol) was added, followed by cyanogen bromide (2.5 mg, 0.024 mmol). The resulting mixture was stirred for 15 min and then purified by reverse phase HPLC (CH₃CN in water, with 0.10% HOAc as a modifier) to provide the title product (10) (6.3 mg, 70%) as a white solid. m/z (ESI, +ve ion)=451.2 [M+H]⁺. ¹H NMR (400 MHz, DMSO-d₆) δ ppm 8.00-8.06 (m, 2H), 7.43-7.50 (m, 3H), 7.30-7.41 (m, 3H), 6.52 (d, J=6.60 Hz, 1H), 6.45 (t, J=6.11 Hz, 1H), 4.30 (d, J=6.11 Hz, 2H), 4.21 (s, 2H), 4.09-4.17 (m, 1H), 3.78 (dt, J=8.07, 6.36 Hz, 1H), 3.63 (dd, J=9.78, 5.62 Hz, 1H), 3.15 (dd, J=9.66, 3.30 Hz, 1H), 1.92 (ddd, J=12.90, 6.42, 3.67 Hz, 1H), 1.65 (ddd, J=12.84, 8.19, 6.11 Hz, 1H), 1.22 (d, J=6.11 Hz, 3H).

### Example 5. 2-(4-(3-(3-Chlorobenzyl)-1,2,4-oxadiazol-5-yl)phenoxy)-N-((3R,5R)-1-cyano-5-methylpyrrolidin-3-yl)propenamide (Compound 11)

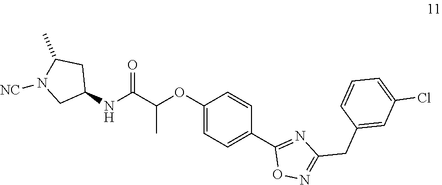 [SMILES: CC(Oc1ccc(-c2nc(Cc3cccc(Cl)c3)no2)cc1)C(=O)N[C@@H]1C[C@@H](C)N(C#N)C1]

### Step A. tert-Butyl 2-(4-(3-(3-chlorobenzyl)-1,2,4-oxadiazol-5-yl)phenoxy)propanoate (11a)

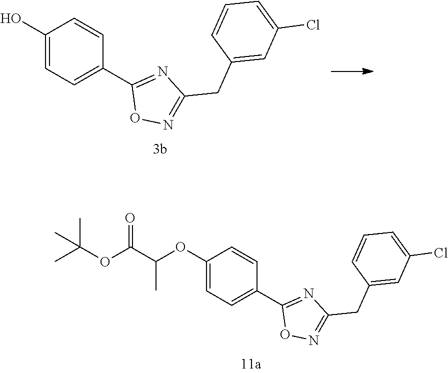 [SMILES: Oc1ccc(-c2nc(Cc3cccc(Cl)c3)no2)cc1 ; CC(Oc1ccc(-c2nc(Cc3cccc(Cl)c3)no2)cc1)C(=O)OC(C)(C)C]

A solution of 3b (7.2 mg, 0.025 mmol) in DMF (0.3 mL) was added K₂CO₃ (6.9 mg, 0.050 mmol) and tert-butyl 2-bromopropanoate (6.3 mg, 0.030 mmol). The reaction was stirred for 3 h at room temperature and then purified by reverse phase HPLC (CH₃CN in water, with 0.1% HOAc as a modifier) to provide the title product (11a) (10 mg, 96%) as a white solid.

### Step B. 2-(4-(3-(3-Chlorobenzyl)-1,2,4-oxadiazol-5-yl)phenoxy)-N-((3R,5R)-1-cyano-5-methylpyrrolidin-3-yl)propenamide (11)

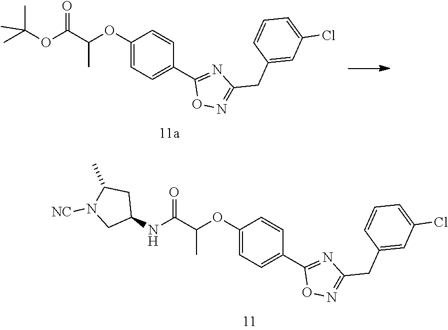 [SMILES: CC(Oc1ccc(-c2nc(Cc3cccc(Cl)c3)no2)cc1)C(=O)OC(C)(C)C ; CC(Oc1ccc(-c2nc(Cc3cccc(Cl)c3)no2)cc1)C(=O)N[C@@H]1C[C@@H](C)N(C#N)C1]

A solution of 11a (5 mg, 0.012 mmol) was dissolved in DCM (0.15 mL) and added TFA (0.15 mL). After stirring at room temperature for 2 h, the reaction was concentrated and redissolved in DMF (0.3 mL). DIPEA (0.0063 mL, 0.036 mmol), HATU (6.8 mg, 0.018 mmol) and 5d (3.3 mg, crude) were added and the reaction was stirred for 10 min at room temperature. The crude material was directly purified by reverse phase HPLC (CH₃CN in water, with 0.1% HOAc as a modifier) to provide the title product (11) (4.0 mg, 68%) as a white solid. m/z (ESI, +ve ion)=466.2 [M+H]⁺.

### Example 6. (E)-3-(4-(3-(3-chlorobenzyl)-1,2,4-oxadiazol-5-yl)phenyl)-N-((3R,5R)-1-cyano-5-methylpyrrolidin-3-yl)-2-methylacrylamide (Compound 13)

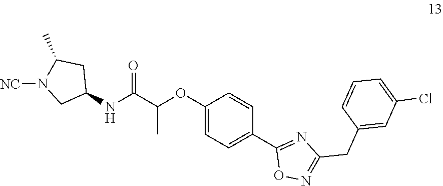 [SMILES: CC(Oc1ccc(-c2nc(Cc3cccc(Cl)c3)no2)cc1)C(=O)N[C@@H]1C[C@@H](C)N(C#N)C1]

### Step A. (E)-4-(3-Ethoxy-2-methyl-3-oxoprop-1-en-1-yl)benzoic acid (13a)

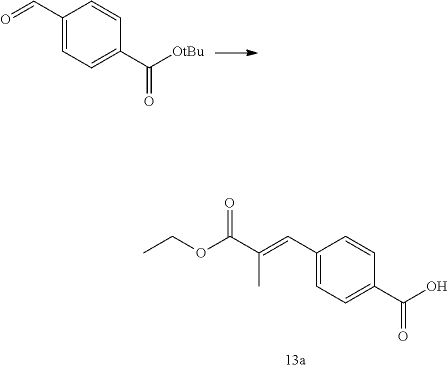 [SMILES: CC(C)(C)OC(=O)c1ccc(C=O)cc1 ; CCOC(=O)/C(C)=C/c1ccc(C(=O)O)cc1]

To a solution of ethyl 2-(diethoxyphosphoryl)propanoate (1.4 g, 5.88 mmol) in dry THF (20 mL) was added NaH (235 mg, 5.88 mmol, 60% dispersion in mineral oil) at 0° C. in portions. After stirring at 0° C. for 0.5 h, tert-butyl 4-formylbenzoate (1.0 g, 4.85 mmol) was added and the reaction was stirred at 20° C. for 4 h. The mixture was quenched with sat. NH₄Cl (50 mL), and extracted with EtOAc (100 mL). The organic layer was washed with brine (50 mL×2), dried, filtered, and concentrated and the crude residue was purified by silica gel column chromatography (petroleum ether:EtOAc=1:1) to afford the title product (13a) (450 mg, 39.8%) as a white solid. m/z (ESI, +ve ion)=235.1 [M+H]⁺.

### Step B. Ethyl (E)-3-(4-(3-(3-chlorobenzyl)-1,2,4-oxadiazol-5-yl)phenyl)-2-methylacrylate (13b)

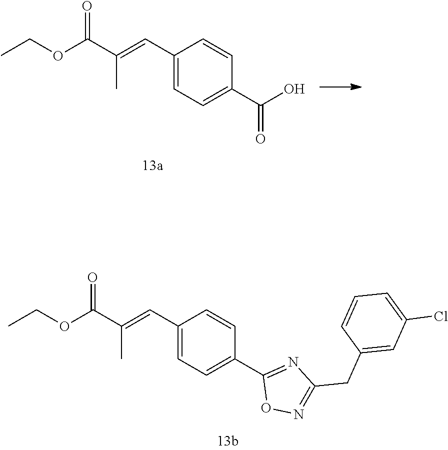 [SMILES: CCOC(=O)/C(C)=C/c1ccc(C(=O)O)cc1 ; CCOC(=O)/C(C)=C/c1ccc(-c2nc(Cc3cccc(Cl)c3)no2)cc1]

To a solution of 13a (400 mg, 1.71 mmol) in NMP (5 mL) was added CDI (332 mg, 2.05 mmol). After stirring at 50° C. for 0.5 h, 3a (315 mg, 1.71 mmol) was added and the reaction was stirred at 120° C. for 2 h. Then the reaction was diluted with EtOAc (30 mL) and washed with brine (30 mL×2). The organic layer was dried, filtered, concentrated, and purified by silica gel column chromatography (petroleum ether:EtOAc=5:1) to afford the title product (13b) (260 mg, 39.7%) as a yellow solid. m/z (ESI, +ve ion)=383.1 [M+H]⁺.

### Step C. (E)-3-(4-(3-(3-Chlorobenzyl)-1,2,4-oxadiazol-5-yl)phenyl)-2-methylacrylic acid (13c)

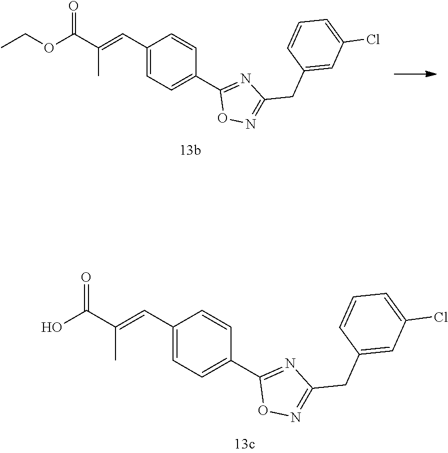 [SMILES: CCOC(=O)/C(C)=C/c1ccc(-c2nc(Cc3cccc(Cl)c3)no2)cc1 ; C/C(=C\c1ccc(-c2nc(Cc3cccc(Cl)c3)no2)cc1)C(=O)O]

To a solution of 13b (130 mg, 0.34 mmol) in THF/H₂O (5 mL, 1:1) was added NaOH (20 mg, 0.51 mmol). The reaction was stirred at 20° C. for 2 h, adjusted to pH=4 with 1 N HCl, and extracted with EtOAc (20 mL). The organic layer was dried, filtered, and concentrated and the residue was purified by prep-TLC (petroleum ether:EtOAc=1:1) to afford the title product (13c) (55 mg, 45.6%) as a white solid. m/z (ESI, −ve ion)=353.0 [M−H]⁻.

### Step D. (E)-3-(4-(3-(3-Chlorobenzyl)-1,2,4-oxadiazol-5-yl)phenyl)-N-((3R,5R)-1-cyano-5-methylpyrrolidin-3-yl)-2-methylacrylamide (13)

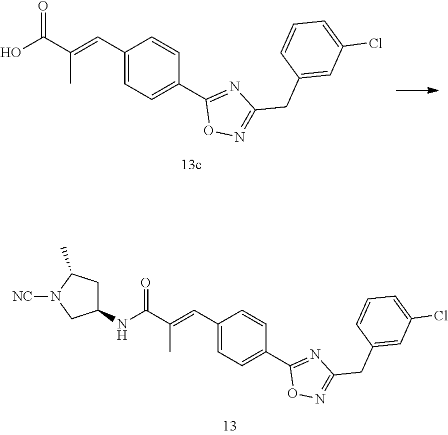 [SMILES: C/C(=C\c1ccc(-c2nc(Cc3cccc(Cl)c3)no2)cc1)C(=O)O ; C/C(=C\c1ccc(-c2nc(Cc3cccc(Cl)c3)no2)cc1)C(=O)N[C@@H]1C[C@@H](C)N(C#N)C1]

The title product (13) was synthesized in a similar procedure as described for the synthesis of Compound 5 in Example 2, Step E. m/z (ESI, +ve ion)=462.3 [M+H]⁺.

### Example 7. (E)-3-(4-(2-(3-Chlorobenzyl)-5-methyloxazol-4-yl)phenyl)-N-((3R,5R)-1-cyano-5-methylpyrrolidin-3-yl)acrylamide (Compound 14)

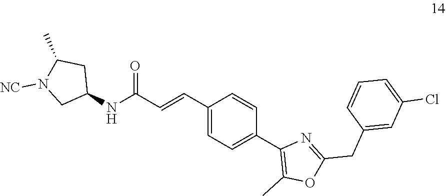 [SMILES: Cc1oc(Cc2cccc(Cl)c2)nc1-c1ccc(/C=C/C(=O)N[C@@H]2C[C@@H](C)N(C#N)C2)cc1]

### Step A. 1-(4-Iodophenyl)propan-1-one (14a)

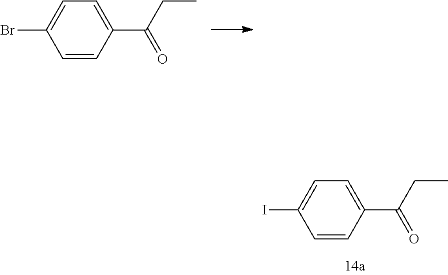

To a stirred solution of 1-(4-bromophenyl)propan-1-one (2 g, 9.39 mmol), CuI (0.179 g, 0.94 mmol) and KI (6.23 g, 37.6 mmol) in 1,4-dioxane (25 mL) under nitrogen at 20° C. was added N₁,N₂-dimethylethane-1,2-diamine (0.17 g, 1.88 mmol). The reaction was heated to reflux for 24 h. After completion, the mixture was filtered. The filtrate was collected and concentrated. The crude residue was purified by silica gel column chromatography (EtOAc:petroleum ether=1:6) to afford the title product (14a) as a white solid (2.1 g, 86%). m/z (ESI, +ve ion)=261.0 [M+H]⁺.

### Step B. 2-Bromo-1-(4-iodophenyl)propan-1-one (14b)

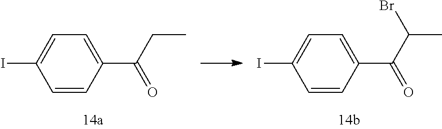

To a solution of 14a (1 g, 3.85 mmol) in DCM (30 mL) was added Br₂ (614.5 mg, 3.85 mmol) at 20° C. and stirred for 12 h. After completion, the mixture was quenched with sat. Na₂SO₃ (30 mL) and extracted with EtOAc (30 mL×3). The combined organic layer was dried with anhydrous Na₂SO₄, filtered and concentrated. The crude residue was purified by silica gel column chromatography (EtOAc:petroleum ether=1:8) to afford the title product (14b) (450 mg, 34.6%) as a pale yellow solid. m/z (ESI, +ve ion)=338.9 [M+H]⁺.

### Step C. 1-(4-Iodophenyl)-1-oxopropan-2-yl 2-(3-chlorophenyl)acetate (14c)

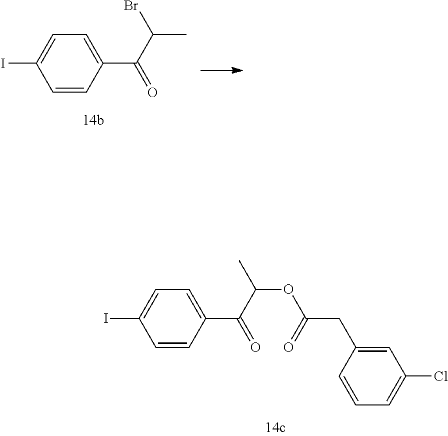 [SMILES: CC(Br)C(=O)c1ccc(I)cc1 ; CC(OC(=O)Cc1cccc(Cl)c1)C(=O)c1ccc(I)cc1]

To a stirred solution of 14b (400 mg, 1.18 mmol) and (3-chlorophenyl)acetic acid (201 mg, 1.18 mmol) in acetone (20 mL) at 20° C. was added DIPEA (457 mg, 3.54 mmol). After stirring for 3 h, the reaction was concentrated and purified by silica gel column chromatography (EtOAc: petroleum ether=1:6) to afford the title product (14c) as a colorless oil (450 mg, 88%). m/z (ESI, +ve ion)=429.0 [M+H]⁺.

### Step D. 2-(3-Chlorobenzyl)-4-(4-iodophenyl)-5-methyloxazole (14d)

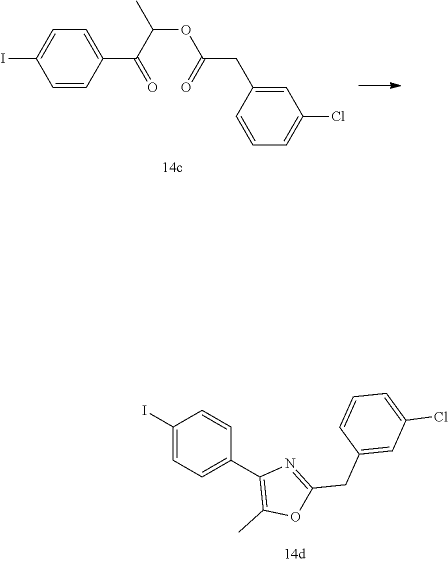 [SMILES: CC(OC(=O)Cc1cccc(Cl)c1)C(=O)c1ccc(I)cc1 ; Cc1oc(Cc2cccc(Cl)c2)nc1-c1ccc(I)cc1]

A solution of 14c (450 mg, 1.05 mmol) and NH₄OAc (405 mg, 5.26 mmol) in acetic acid (10 mL) was heated at 120° C. for 4 h. After completion, the mixture was diluted with water (50 mL) and extracted with EtOAc (30 mL×3). The combined organic layer was dried with anhydrous Na₂SO₄, filtered and concentrated. The crude product was purified by silica gel column chromatography (EtOAc:petroleum ether=1:6) to afford the title product (14d) as a white solid (390 mg, 85%). m/z (ESI, +ve ion)=410.0 [M+H]⁺.

### Step E. (E)-3-(4-(2-(3-Chlorobenzyl)-5-methyloxazol-4-yl)phenyl)acrylic acid (14e)

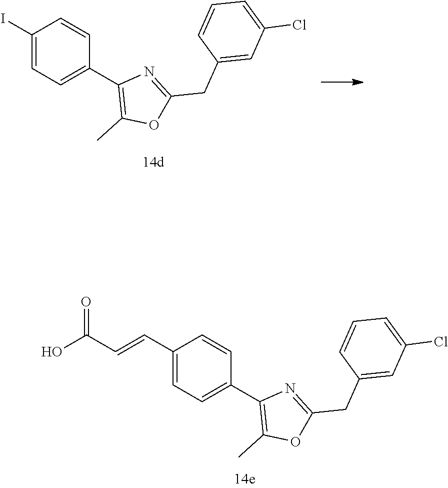 [SMILES: Cc1oc(Cc2cccc(Cl)c2)nc1-c1ccc(I)cc1 ; Cc1oc(Cc2cccc(Cl)c2)nc1-c1ccc(/C=C/C(=O)O)cc1]

The title product (14e) was synthesized using similar procedures for the synthesis of Compound 5 as described in Example 2, Steps B and C. m/z (ESI, +ve ion)=354.0 [M+H]⁺.

### Step F. (E)-3-(4-(2-(3-Chlorobenzyl)-5-methyloxazol-4-yl)phenyl)-N-((3R,5R)-1-cyano-5-methylpyrrolidin-3-yl)acrylamide (14)

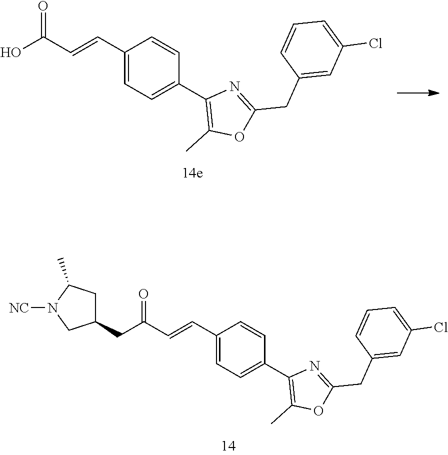 [SMILES: Cc1oc(Cc2cccc(Cl)c2)nc1-c1ccc(/C=C/C(=O)O)cc1 ; Cc1oc(Cc2cccc(Cl)c2)nc1-c1ccc(/C=C/C(=O)C[C@@H]2C[C@@H](C)N(C#N)C2)cc1]

The title product (14) was synthesized using similar procedures as described for the synthesis of Compound 5 in Example 2, Steps D to E. m/z (ESI, +ve ion)=461.3 [M+H]⁺.

### Example 8. (E)-3-(4-(2-(3-Chlorobenzyl)-5-methyloxazol-4-yl)phenyl)-N-((3R,5R)-5-methylpyrrolidin-3-yl)acrylamide (Compound 15)

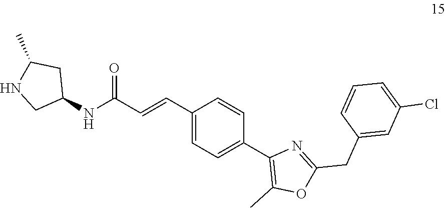 [SMILES: Cc1oc(Cc2cccc(Cl)c2)nc1-c1ccc(/C=C/C(=O)N[C@H]2CN[C@H](C)C2)cc1]

### Step A. tert-Butyl (2R,4R)-4-((E)-3-(4-(2-(3-chlorobenzyl)-5-methyloxazol-4-yl)phenyl)acrylamido)-2-methylpyrrolidine-1-carboxylate (15a)

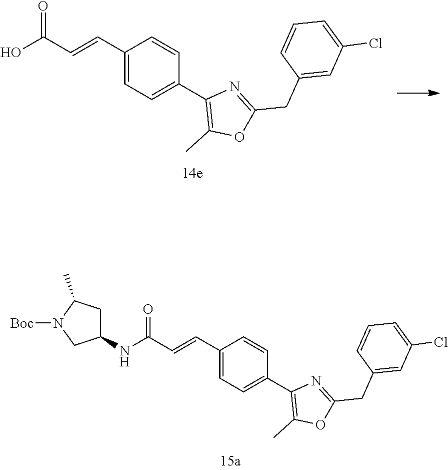 [SMILES: Cc1oc(Cc2cccc(Cl)c2)nc1-c1ccc(/C=C/C(=O)O)cc1 ; Cc1oc(Cc2cccc(Cl)c2)nc1-c1ccc(/C=C/C(=O)N[C@@H]2C[C@@H](C)N(C(=O)OC(C)(C)C)C2)cc1]

A solution of 14e (24.7 mg, 0.07 mmol), tert-butyl (2R,4R)-4-amino-2-methylpyrrolidine-1-carboxylate (16.8 mg, 0.084 mmol), HATU (35 mg, 0.092 mmol), DIPEA (0.024 mL, 0.14 mmol) in DMF (0.5 mL) was stirred at room temperature for 1 h. The mixture was directly purified by reverse phase HPLC (CH₃CN in water, with 0.1% HOAc as a modifier) to provide the title product (15a) (24 mg, 64%) as a white solid. m/z (ESI, +ve ion)=480.3 [M-tBu]⁺.

### Step B. (E)-3-(4-(2-(3-Chlorobenzyl)-5-methyloxazol-4-yl)phenyl)-N-((3R,5R)-5-methylpyrrolidin-3-yl)acrylamide (15)

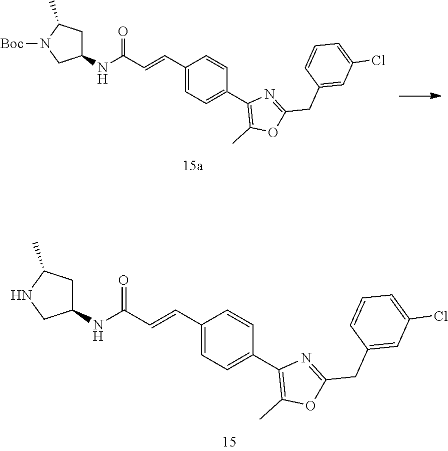 [SMILES: Cc1oc(Cc2cccc(Cl)c2)nc1-c1ccc(/C=C/C(=O)N[C@@H]2C[C@@H](C)N(C(=O)OC(C)(C)C)C2)cc1 ; Cc1oc(Cc2cccc(Cl)c2)nc1-c1ccc(/C=C/C(=O)N[C@H]2CN[C@H](C)C2)cc1]

To a solution of 15a (21.4 mg, 0.04 mmol) in DCM (1 mL) was added TFA (1 mL). After stirring for 50 min at room temperature, the reaction was concentrated and lyophilized to provide the title product (15) (18.8 mg, 86%, TFA salt) as a white solid. m/z (ESI, +ve ion)=436.2 [M+H]⁺.

Compound 16 was synthesized from 3a and 4-(2-methoxy-2-oxoethyl)benzoic acid using similar procedures for the synthesis of Compound 13 as described in Example 6, Steps B to D. T₃P was used as the coupling reagent instead of HATU in the last step.

Compound 17 was synthesized in a similar procedure as described for the synthesis of Compound 5 in Example 2 using 3-iodobenzoic acid.

### Example 9. 4-((4-(3-(3-Chlorobenzyl)-1,2,4-oxadiazol-5-yl)benzyl)oxy)-N,N-dimethylbenzamide (Compound 18)

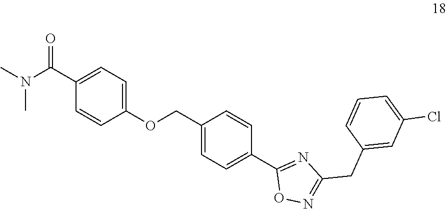 [SMILES: CN(C)C(=O)c1ccc(OCc2ccc(-c3nc(Cc4cccc(Cl)c4)no3)cc2)cc1]

### Step A. Methyl 4-(3-(3-chlorobenzyl)-1,2,4-oxadiazol-5-yl)benzoate (18)

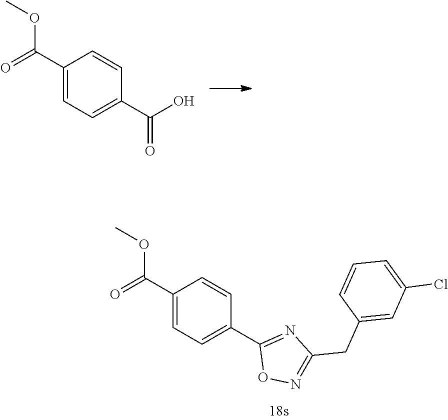 [SMILES: COC(=O)c1ccc(C(=O)O)cc1 ; COC(=O)c1ccc(-c2nc(Cc3cccc(Cl)c3)no2)cc1]

To a solution of 4-(methoxycarbonyl) benzoic acid (5.0 g, 27.8 mmol) in NMP (100 mL) was added CDI (5.0 g, 30.5 mmol). After heating at 50° C. for 2 h, 3a (5.1 g, 27.8 mmol) was added to the above solution. The mixture was stirred at 120° C. for 16 h. After cooling down, the reaction was diluted with EtOAc (200 mL) and washed with H₂O (500 mL). The aqueous layer was extracted with EtOAc (200 mL). The combined organic layer was washed with H₂O (100 mL×2), brine (100 mL), dried over anhydrous Na₂SO₄, filtered, and concentrated to give a crude residue, which was purified by silica gel column chromatography (EtOAc:petroleum ether=1:5) to afford the title product (18a) (4.9 g, 53.6%) as a white solid. m/z (ESI, +ve ion)=329.7 [M+H]⁺.

### Step B. (4-(3-(3-Chlorobenzyl)-1,2,4-oxadiazol-5-yl)phenyl)methanol (18b)

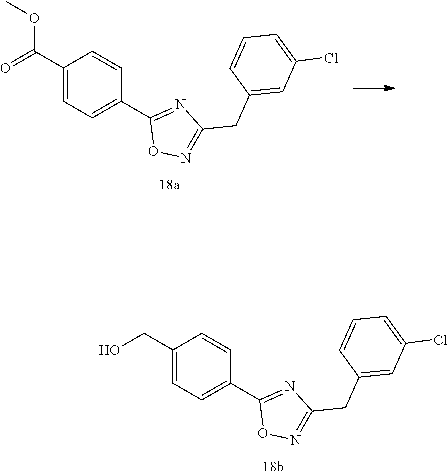 [SMILES: COC(=O)c1ccc(-c2nc(Cc3cccc(Cl)c3)no2)cc1 ; OCc1ccc(-c2nc(Cc3cccc(Cl)c3)no2)cc1]

To a solution of 18a (2.0 g, 6.1 mmol) in THF (30 mL) at −78° C. was added 1 M diisobutylaluminum hydride (18.3 mL, 0.3 mmol) slowly. After stirring for 4 h at the same temperature, the reaction was quenched with 1 N HCl to adjust to pH=4-5, and extracted with EtOAc (50 mL×3). The organic layer was washed with H₂O (20 mL), brine (20 mL), dried over Na₂SO₄, filtered and concentrated in vacuo to give a crude residue, which was purified by silica gel column chromatography (petroleum ether:EtOAc=1:1) to afford the title product (18b) (1.2 g, 62.3%) as a white solid. m/z (ESI, +ve ion)=301.7 [M+H]⁺.

### Step C. 5-(4-(Bromomethyl)phenyl)-3-(3-chlorobenzyl)-1,2,4-oxadiazole (18c)

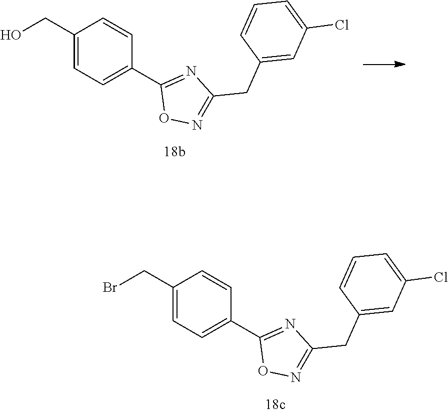 [SMILES: OCc1ccc(-c2nc(Cc3cccc(Cl)c3)no2)cc1 ; Clc1cccc(Cc2noc(-c3ccc(CBr)cc3)n2)c1]

To a solution of POBr₃ (571 mg, 2.0 mmol) in DCM (10 mL) was added DMF (5.0 mL) at 0° C. The mixture was stirred at 0° C. for 0.25 h, then 18b (500 mg, 1.66 mmol) was added. After stirring at 0° C. for 1 h, the mixture was quenched with 5% aqueous NaHCO₃ to adjust to pH=7-8, and extracted with EtOAc (30 mL×3). The combined organic layer was washed with H₂O (15 mL×2), brine (15 mL), dried over Na₂SO₄, filtered, and concentrated in vacuo to give a crude residue, which was purified by reverse phase HPLC (70% CH₃CN in H₂O, with 0.05% formic acid as a modifier) to afford the title product (18c) (350 mg, 57.3%) as a white solid. m/z (ESI, +ve ion)=365.0 [M+H]⁺.

### Step D. 4-((4-(3-(3-Chlorobenzyl)-1,2,4-oxadiazol-5-yl)benzyl)oxy)-N,N-dimethylbenzamide (18)

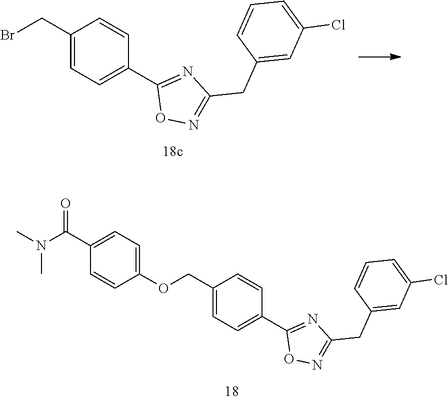 [SMILES: Clc1cccc(Cc2noc(-c3ccc(CBr)cc3)n2)c1 ; CN(C)C(=O)c1ccc(OCc2ccc(-c3nc(Cc4cccc(Cl)c4)no3)cc2)cc1]

To a mixture of 4-hydroxy-N,N-dimethylbenzamide (9.1 mg, 0.055 mmol) and K₂CO₃ (22.8 mg, 0.17 mmol) in DMF (1.3 mL) was added 18c (20 mg, 0.055 mmol). The reaction was stirred at 50° C. for 2 h, diluted with H₂O and EtOAc, and extracted with EtOAc (×3). The combined organic phases were washed with water, brine (×2), dried with Na₂SO₄, filtered and concentrated under reduced pressure. The crude reside was purified by silica gel column chromatography (petroleum ether: EtOAc=1:1) to afford the title product (18) (22 mg, 89%) as a white solid. m/z (ESI, +ve ion)=448.1 [M+H]⁺. ¹H NMR (600 MHz, CDCl₃) δ ppm 8.14 (d, J=8.07 Hz, 2H), 7.58 (d, J=8.07 Hz, 2H), 7.39-7.43 (m, 3H), 7.21-7.33 (m, 3H), 6.98 (d, J=8.44 Hz, 2H), 5.18 (s, 2H), 4.13 (s, 2H), 3.07 (br s, 6H).

Compound 19 was synthesized in a similar procedure from 10c as described for the synthesis of Compound 15 in Example 8, Step A.

### Example 10. 1-(4-((4-(3-(3-Chlorobenzyl)-1,2,4-oxadiazol-5-yl)benzyl)oxy)phenyl)-N,N-dimethylmethanamine (Compound 20)

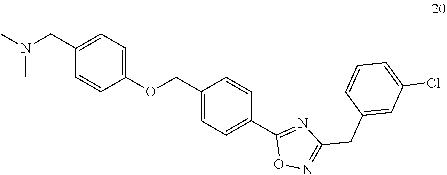 [SMILES: CN(C)Cc1ccc(OCc2ccc(-c3nc(Cc4cccc(Cl)c4)no3)cc2)cc1]

### Step A. 4-((4-(3-(3-Chlorobenzyl)-1,2,4-oxadiazol-5-yl)benzyl)oxy)benzaldehyde (20a)

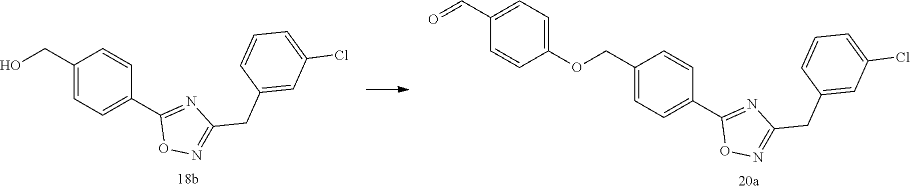 [SMILES: OCc1ccc(-c2nc(Cc3cccc(Cl)c3)no2)cc1 ; O=Cc1ccc(OCc2ccc(-c3nc(Cc4cccc(Cl)c4)no3)cc2)cc1]

To a solution of 18b (200 mg, 0.55 mmol) and 4-hydroxybenzaldehyde (67 mg, 0.55 mmol) in DMF (10 mL) was added K₂CO₃ (152 mg, 1.1 mmol) at 25° C. The reaction was heated to 55° C. and stirred for 1 h. After completion, the reaction was quenched with water (50 mL) and extracted with EtOAc (50 mL×3). The combined organic layer was washed with H₂O (50 mL×2), brine (30 mL), dried over anhydrous Na₂SO₄, filtered and concentrated in vacuo to give a crude residue, which was purified by reverse phase HPLC (65% CH₃CN in H₂O, with 0.05% formic acid as a modifier) to afford the title product (20a) (150 mg, 67%) as a white solid. m/z (ESI, +ve ion)=405.0 [M+H]⁺.

### Step B. 1-(4-((4-(3-(3-Chlorobenzyl)-1,2,4-oxadiazol-5-yl)benzyl)oxy)phenyl)-N,N-dimethylmethanamine (20)

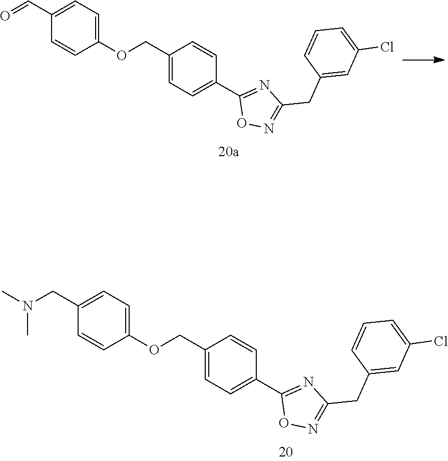 [SMILES: O=Cc1ccc(OCc2ccc(-c3nc(Cc4cccc(Cl)c4)no3)cc2)cc1 ; CN(C)Cc1ccc(OCc2ccc(-c3nc(Cc4cccc(Cl)c4)no3)cc2)cc1]

To a mixture of 20a (20 mg, 0.05 mmol), dimethylamine hydrochloride (2.7 mg, 0.06 mmol) and Et₃N (15 mg, 0.15 mmol) in DCM (3 mL) under N₂ was added NaBH(OAc)₃ (16 mg, 0.075 mmol) at 0° C. After stirring at 0° C. for 1 h, the reaction was raised to room temperature and stirred for 12 h. After completion, the mixture was quenched with H₂O (10 mL), and extracted with EtOAc (10 mL×3). The combined organic layer was washed with H₂O (10 mL), brine (10 mL), dried over anhydrous Na₂SO₄, filtered and concentrated in vacuo to give a crude residue, which was purified by reverse phase HPLC (65% CH₃CN in H₂O, with 0.05% formic acid as a modifier) to afford the title product (20) (4.3 mg, 19.8%) as a white solid. m/z (ESI, +ve ion)=434.2 [M+H]⁺. ¹H NMR (400 MHz, CD₃OD) δ ppm 8.11 (d, J=8.3 Hz, 2H), 7.64 (d, J=8.2 Hz, 2H), 7.39 (s, 1H), 7.10-7.35 (m, 5H), 7.03 (d, J=8.7 Hz, 2H), 5.20 (s, 2H), 4.14 (s, 2H), 3.75 (s, 2H), 2.46 (s, 6H).

### Example 11. 4-((4-(2-(3-Chlorobenzyl)-5-methyloxazol-4-yl)benzyl)oxy)-N,N-dimethylbenzamide (Compound 21)

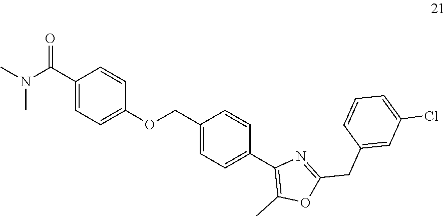 [SMILES: Cc1oc(Cc2cccc(Cl)c2)nc1-c1ccc(COc2ccc(C(=O)N(C)C)cc2)cc1]

### Step A. (4-((Benzyloxy)methyl)phenyl)methanol

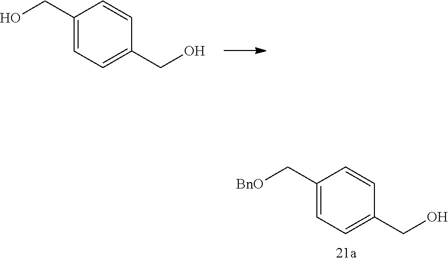 [SMILES: OCc1ccc(CO)cc1 ; OCc1ccc(COCc2ccccc2)cc1]

A solution of [4-(hydroxymethyl)phenyl]methanol (50 g, 0.36 mol) in THF (350 mL) was cooled to 0° C. and sodium hydride (17.6 g, 0.44 mol, 60% dispersion in mineral oil) was added. After stirring for 1 h at 0° C., TBAI (8.02 g, 21.7 mmol) and BnBr (51.4 mL, 0.43 mol) were added. The resulting mixture was stirred at 25° C. for 18 h, quenched with ice-water (100 mL) and extracted with EtOAc (100 mL×3). The combined organic layer was washed with water (15 mL), brine (150 mL), dried over Na₂SO₄, filtered and concentrated in vacuo to afford a crude residue, which was purified by silica gel column chromatography (EtOAc:petroleum ether=1:10) to afford the title product (21a) (38 g, 90%) as a yellow oil. m/z (ESI, +ve ion)=251.1 [M+Na]+.

### Step B. 4-((Benzyloxy)methyl)benzaldehyde (21b)

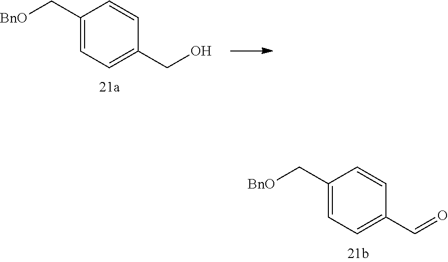 [SMILES: OCc1ccc(COCc2ccccc2)cc1 ; O=Cc1ccc(COCc2ccccc2)cc1]

To a mixture of 21a (20.0 g, 0.088 mol) in THF (250 mL) was added MnO₂ (53 g, 0.61 mol) at 25° C. The reaction was stirred at 70° C. for 4 h, cooled down, and filtered. The filtrate was concentrated in vacuo to afford the title product (21b) (14.6 g, 74%) as yellow oil, which was used in the next step without further purification. m/z (ESI, +ve ion)=227.1 [M+H]⁺.

### Step C. 1-(4-((Benzyloxy)methyl)phenyl)propan-1-ol (21c)

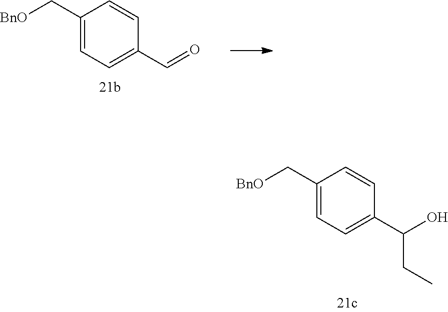 [SMILES: O=Cc1ccc(COCc2ccccc2)cc1 ; CCC(O)c1ccc(COCc2ccccc2)cc1]

To a mixture of 21b (14.5 g, 64.1 mmol) in anhydrous THF (70 mL) was added ethyl magnesium bromide (35.3 mL, 70.5 mmol) at 0° C. After stirring at 0° C. for 1.5 h, the reaction was quenched with ice-cold saturated NH₄Cl (200 mL) and extracted with EtOAc (150 mL×3). The combined organic layer was washed with water (100 mL), brine (100 mL), dried over Na₂SO₄, filtered and concentrated in vacuo to afford a crude residue. The residue was purified by silica gel column chromatography (petroleum ether:EtOAc=4:1) to afford the title product (21c) (11.9 g, 85%) as a yellow oil. m/z (ESI, +ve ion)=279.1 [M+Na]+.

### Step D. 1-(4-((Benzyloxy)methyl)phenyl)-2-hydroxypropan-1-one (21d)

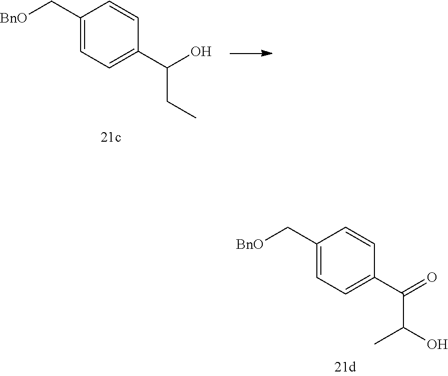 [SMILES: CCC(O)c1ccc(COCc2ccccc2)cc1 ; CC(O)C(=O)c1ccc(COCc2ccccc2)cc1]

To a solution of 21c (5.0 g, 19.5 mmol) in DMSO/dioxane (100 mL, 2:1) was added I₂ (1.98 g, 7.8 mmol) at 25° C. After stirring for 1 h, IBX (16.38 g, 58.5 mmol) was added and the reaction was heated at 80° C. for 24 h. After cooling down, the reaction mixture was poured into water (50 mL) and extracted with EtOAc (25 mL×2). The combined organic layer was dried over Na₂SO₄, filtered, concentrated and purified by silica gel column chromatography (petroleum ether:EtOAc=10:1) to afford the title product (21d) (1.74 g, 29.7%) as a colorless oil. m/z (ESI, +ve ion)=271.1 [M+H]⁺.

### Step E. 1-(4-((Benzyloxy)methyl)phenyl)-1-oxopropan-2-yl 2-(3-chlorophenyl)acetate (21e)

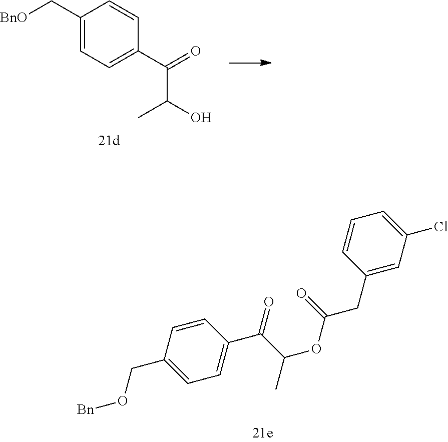 [SMILES: CC(O)C(=O)c1ccc(COCc2ccccc2)cc1 ; CC(OC(=O)Cc1cccc(Cl)c1)C(=O)c1ccc(COCc2ccccc2)cc1]

A solution of 21d (3.1 g, 11.4 mmol), (3-chlorophenyl)acetic acid (3.9 g, 22.8 mmol), 4-dimethylaminopyridine (1.4 g, 11.4 mmol) and N-(3-dimethylaminopropyl)-N′-ethylcarbodiimide hydrochloride (2.84 g, 14.8 mmol) in DCM (25 mL) was stirred at 25° C. for 5 h. The reaction was diluted with water and extracted with EtOAc (20 mL×3). The combined organic layer was dried over Na₂SO₄, filtered, concentrated and purified by silica gel column chromatography (EtOAc:petroleum ether=1:50) to afford the title product (21e) (3 g, 56%) as a colorless oil. m/z (ESI, +ve ion)=445.0 [M+Na]+.

### Step F. 4-(4-((Benzyloxy)methyl)phenyl)-2-(3-chlorobenzyl)-5-methyloxazole (21f)

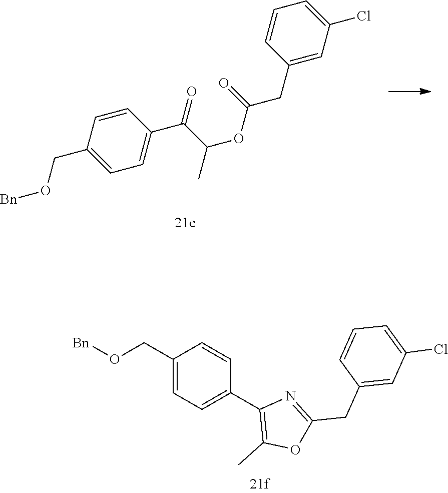 [SMILES: CC(OC(=O)Cc1cccc(Cl)c1)C(=O)c1ccc(COCc2ccccc2)cc1 ; Cc1oc(Cc2cccc(Cl)c2)nc1-c1ccc(COCc2ccccc2)cc1]

To a stirred solution of 21e (2.3 g, 5.4 mmol) in acetic acid (40 mL) was added NH₄OAc (6.24 g, 81 mmol). The reaction was stirred at 120° C. for 2 h. After completion, the reaction was diluted with H₂O (15 mL) and extracted with EtOAc (20 mL×3). The combined organic layer was dried over Na₂SO₄, filtered, concentrated and purified by silica gel column chromatography (EtOAc:petroleum ether=1:20) to afford the title product (21f) (1.6 g, 67%) as a white solid. m/z (ESI, +ve ion)=404.1 [M+H]⁺.

### Step G. (4-(2-(3-Chlorobenzyl)-5-methyloxazol-4-yl)phenyl)methanol (21g)

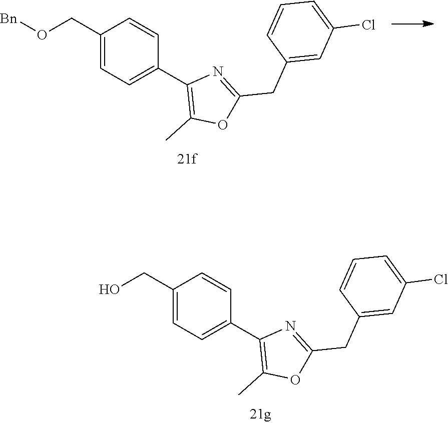 [SMILES: Cc1oc(Cc2cccc(Cl)c2)nc1-c1ccc(COCc2ccccc2)cc1 ; Cc1oc(Cc2cccc(Cl)c2)nc1-c1ccc(CO)cc1]

To a stirred solution of 21f (1.5 g, 3.7 mmol) in DCM (20 mL) was added BCI₃ (1 M in DCM, 7.5 mL) dropwise at 0° C. The reaction mixture was stirred at 25° C. for an additional 2 h and extracted with EtOAc (5 mL×3). The combined organic layer was dried over Na₂SO₄, filtered, concentrated and purified by silica gel column chromatography (EtOAc:petroleum ether=1:50) to afford the title product (21g) (0.61 g, 49%) as a white solid. m/z (ESI, +ve ion)=314.1 [M+H]⁺.

### Step H. 4-(4-(Bromomethyl)phenyl)-2-(3-chlorobenzyl)-5-methyloxazole (21h)

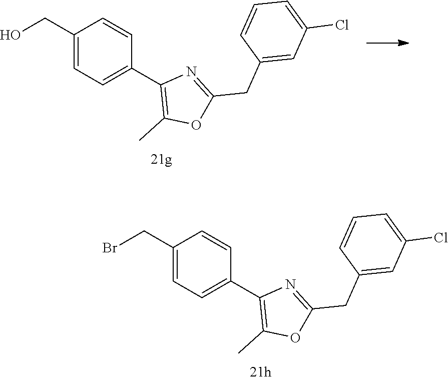 [SMILES: Cc1oc(Cc2cccc(Cl)c2)nc1-c1ccc(CO)cc1 ; Cc1oc(Cc2cccc(Cl)c2)nc1-c1ccc(CBr)cc1]

To a solution of 21g (1 g, 4.2 mol) in DCM (50 mL) was added PBr₃ (0.57 g, 2.1 mol) dropwise at 0° C. After stirring for 2 h at room temperature, the reaction was concentrated to provide the crude title product (21h) (1 g), which was directly used in the next step without further purification. m/z (ESI, +ve ion)=376.0 [M+H]⁺.

### Step I. 4-((4-(2-(3-Chlorobenzyl)-5-methyloxazol-4-yl)benzyl)oxy)-N,N-dimethylbenzamide (21)

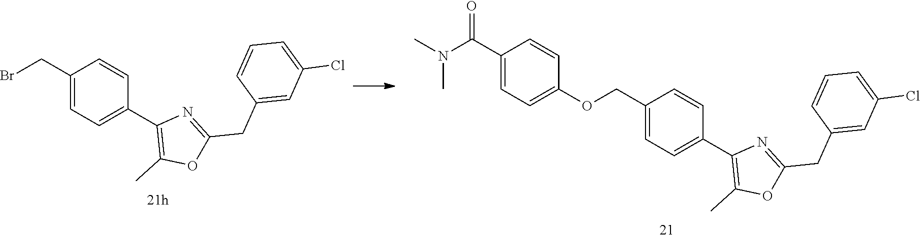 [SMILES: Cc1oc(Cc2cccc(Cl)c2)nc1-c1ccc(CBr)cc1 ; Cc1oc(Cc2cccc(Cl)c2)nc1-c1ccc(COc2ccc(C(=O)N(C)C)cc2)cc1]

To a stirred solution of 4-hydroxy-N,N-dimethylbenzamide (7.9 mg, 0.048 mmol) in DMF (1 mL) was added K₂CO₃ (19.8 mg, 0.14 mmol) and 21h (18 mg, 0.048 mmol) at room temperature. The reaction was heated at 50° C. for 2 h. After cooling down, the reaction was diluted with water and EtOAc and extracted with EtOAc (×3). The combined organic layer was washed with water, brine (×2), dried over Na₂SO₄, filtered and concentrated under reduced pressure. The crude residue was purified by silica gel column chromatography (gradient elution, 20-90% EtOAc in hexanes) to provide an impure material, which was repurified (gradient elution, 10-60% EtOAc in acetone) to provide the title product (21) (15 mg, 68%) as a white solid. m/z (ESI, +ve ion)=461.4 [M+H]⁺.

### Example 12. 4-((4-(2-(3-Chlorobenzyl)-5-methyloxazol-4-yl)phenoxy)methyl)-N,N-dimethylbenzamide (Compound 22)

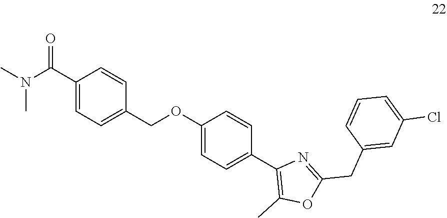 [SMILES: Cc1oc(Cc2cccc(Cl)c2)nc1-c1ccc(OCc2ccc(C(=O)N(C)C)cc2)cc1]

### Step A. 4-(2-(3-Chlorobenzyl)-5-methyloxazol-4-yl)phenol (22b)

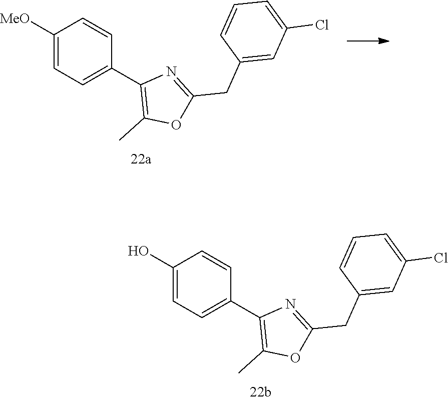 [SMILES: COc1ccc(-c2nc(Cc3cccc(Cl)c3)oc2C)cc1 ; Cc1oc(Cc2cccc(Cl)c2)nc1-c1ccc(O)cc1]

To a solution of 22a (420 mg, 1.34 mmol) in anhydrous DCM (5 mL) was added boron tribromide (1.68 g, 6.71 mmol) at −78° C. under nitrogen. The mixture was allowed to warm to 20° C. and stirred for 3 h. After completion, the reaction was quenched with water (5 mL) and extracted with EtOAc (20 mL×3). The combined organic layer was dried, filtered, and concentrated to afford a crude residue, which was purified by reverse phase HPLC (40-45% CH₃CN in H₂O, with 0.1% formic acid as a modifier) to afford the title product (22b) (105 mg, 26%) as a white solid. m/z (ESI, +ve ion)=300.1 [M+H]⁺.

### Step B. tert-Butyl 4-((4-(2-(3-chlorobenzyl)-5-methyloxazol-4-yl)phenoxy)methyl)benzoate (22c)

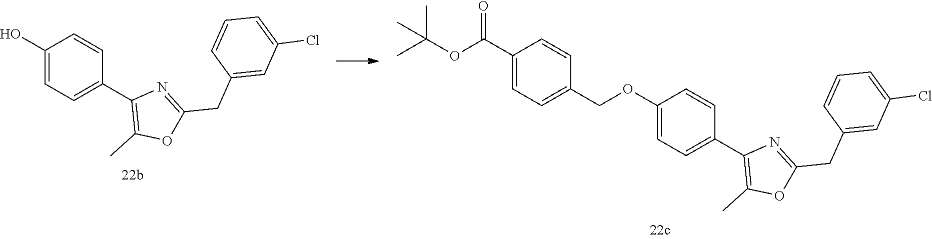 [SMILES: Cc1oc(Cc2cccc(Cl)c2)nc1-c1ccc(O)cc1 ; Cc1oc(Cc2cccc(Cl)c2)nc1-c1ccc(OCc2ccc(C(=O)OC(C)(C)C)cc2)cc1]

To a solution of 22b (50 mg, 0.17 mmol) in DMF (0.5 mL) was added Cs₂CO₃ (70.7 mg, 0.22 mmol) and tert-butyl 4-(bromomethyl)benzoate (49.8 mg, 0.18 mmol). After 1 h, additional Cs₂CO₃ (71 mg) and benzyl bromide (49.8 mg) were added and the addition was repeated after another hour of stirring. After completion, the reaction was quenched by sat. NH₄Cl, extracted with EtOAc (×3), and washed with water and brine (×2). The combined organic layer was dried over Na₂SO₄, filtered and concentrated under reduced pressure. The resulting crude residue was purified by silica gel column chromatography (gradient elution, 0-20% EtOAc in hexanes) to provide the title product (22c) (73.6 mg, 90%) as a colorless gum. m/z (ESI, +ve ion)=490.2 [M+H]⁺.

### Step C. 4-((4-(2-(3-Chlorobenzyl)-5-methyloxazol-4-yl)phenoxy)methyl)benzoic acid (22d)

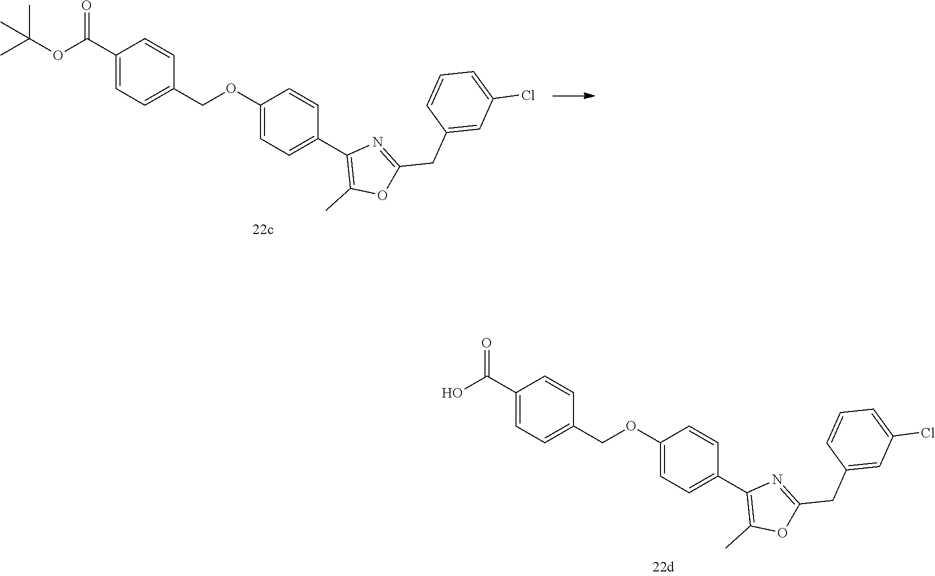 [SMILES: Cc1oc(Cc2cccc(Cl)c2)nc1-c1ccc(OCc2ccc(C(=O)OC(C)(C)C)cc2)cc1 ; Cc1oc(Cc2cccc(Cl)c2)nc1-c1ccc(OCc2ccc(C(=O)O)cc2)cc1]

To a stirred solution of 22c (73.6 mg, 0.15 mmol) in DCM (2.5 mL) was added TFA (1 mL). After stirring for 1 h at room temperature, the reaction was concentrated under reduced pressure. The crude residue was purified by silica gel column chromatography (gradient elution, 1-5% MeOH in DCM) to provide the title product (22d) (76 mg, quantitative) as an off-white solid. m/z (ESI, +ve ion)=434.2 [M+H]⁺.

### Step D. 4-((4-(2-(3-Chlorobenzyl)-5-methyloxazol-4-yl)phenoxy)methyl)-N,N-dimethylbenzamide (22)

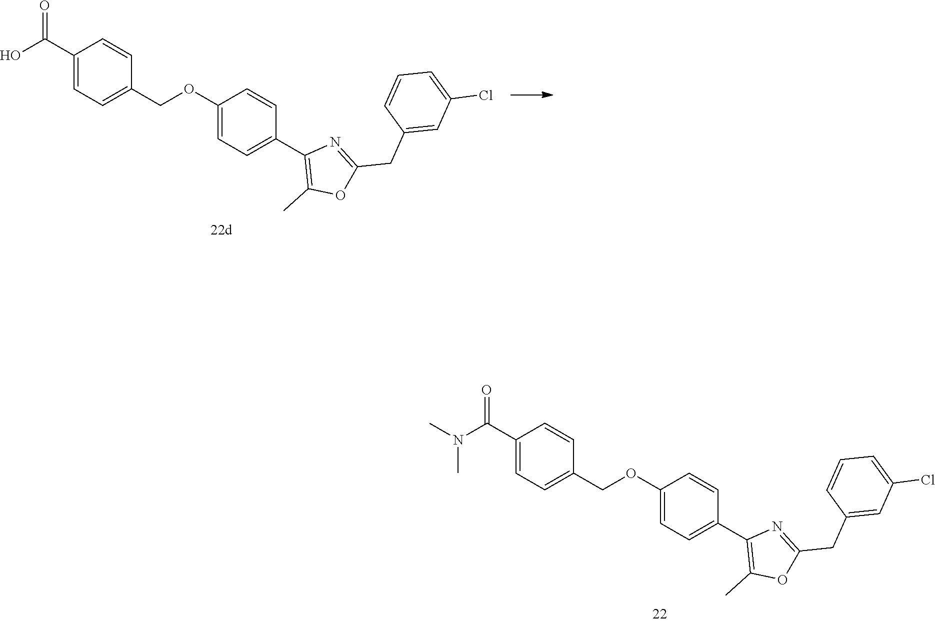 [SMILES: Cc1oc(Cc2cccc(Cl)c2)nc1-c1ccc(OCc2ccc(C(=O)O)cc2)cc1 ; Cc1oc(Cc2cccc(Cl)c2)nc1-c1ccc(OCc2ccc(C(=O)N(C)C)cc2)cc1]

To a stirred solution of 22d (35 mg, 0.081 mmol) and dimethylamine (0.048 mL, 0.097 mmol) in DMF (1.0 mL) were added DIPEA (0.042 mL, 0.24 mmol) and HATU (36.8 mg, 0.097 mmol). After stirring for 3 h, the reaction was diluted with water, and extracted with EtOAc (×3). The combined organic layer was washed with water, brine (×2), dried over Na₂SO₄, filtered and concentrated under reduced pressure. The crude residue was purified by silica gel column chromatography (gradient elution, 0.5 to 2.5% MeOH in DCM) to provide the title product (22) (11.6 mg, 31%) as a white solid. m/z (ESI, +ve ion)=461.2 [M+H]⁺. ¹H NMR (600 MHz, CDCl₃) δ ppm 7.53-7.61 (m, 2H), 7.43-7.51 (m, 4H), 7.35 (s, 1H), 7.22-7.30 (m, 3H), 7.00-7.04 (m, 2H), 5.13 (s, 2H), 4.08 (s, 2H), 3.13 (br s, 3H), 3.00 (s, 3H), 2.46 (s, 3H).

### Example 13. 3-((4-(2-(3-Chlorobenzyl)-5-methyloxazol-4-yl)phenoxy)methyl)-N,N,1-trimethyl-1H-pyrazole-5-carboxamide (Compound 23)

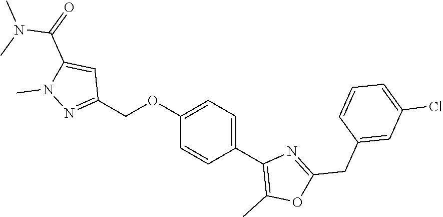 [SMILES: Cc1oc(Cc2cccc(Cl)c2)nc1-c1ccc(OCc2cc(C(=O)N(C)C)n(C)n2)cc1]

### Step A. 3-((4-(2-(3-Chlorobenzyl)-5-methyloxazol-4-yl)phenoxy)methyl)-1-methyl-1H-pyrazole-5-carboxylic acid (23b)

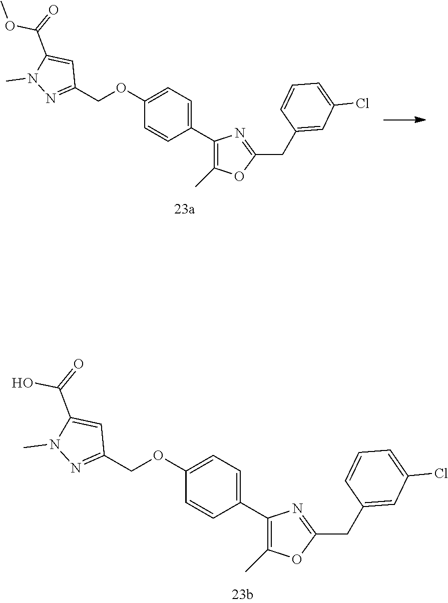 [SMILES: COC(=O)c1cc(COc2ccc(-c3nc(Cc4cccc(Cl)c4)oc3C)cc2)nn1C ; Cc1oc(Cc2cccc(Cl)c2)nc1-c1ccc(OCc2cc(C(=O)O)n(C)n2)cc1]

To a cloudy solution of 23a (prepared in similar procedures as 22c, 66 mg, 0.15 mmol) in THF/water/MeOH (1.0 mL, 2:1:1) was added LiOH hydrate (12.3 mg, 0.29 mmol) at room temperature. After stirring for 1 h, the reaction was diluted with DCM and water, acidified by 1 N HCl, and extracted with DCM (×3). The combined organic layer was washed with brine, dried over Na₂SO₄, filtered and concentrated under reduced pressure. The crude residue was purified by silica gel column chromatography (gradient elution, 2 to 5% MeOH in DCM) to provide the title product (23b) (61 mg, 95%) as a white solid. m/z (ESI, +ve ion)=438.3 [M+H]⁺.

### Step B. 3-((4-(2-(3-Chlorobenzyl)-5-methyloxazol-4-yl)phenoxy)methyl)-N,N,1-trimethyl-1H-pyrazole-5-carboxamide (23)

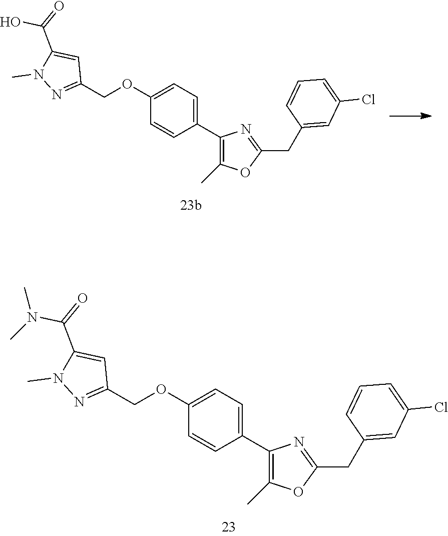 [SMILES: Cc1oc(Cc2cccc(Cl)c2)nc1-c1ccc(OCc2cc(C(=O)O)n(C)n2)cc1 ; Cc1oc(Cc2cccc(Cl)c2)nc1-c1ccc(OCc2cc(C(=O)N(C)C)n(C)n2)cc1]

The title compound (23) was synthesized from 23b in a similar procedure as the synthesis for Compound 22 as described in Example 12, Step D. m/z (ESI, +ve ion)=465.1 [M+H]⁺. ¹H NMR (600 MHz, CDCl₃) δ ppm 7.54-7.64 (m, 2H), 7.32-7.38 (m, 1H), 7.22-7.31 (m, 3H), 6.99-7.06 (m, 2H), 6.75 (s, 1H), 5.08 (s, 2H), 4.08 (s, 2H), 3.96 (s, 3H), 3.36 (s, 3H), 3.11 (s, 3H), 2.47 (s, 3H).

Compounds 24, 25, 26 (using methyl 2-(chloromethyl)pyrimidine-5-carboxylate), and 28 were synthesized in similar procedures as described for the synthesis of Compound 23 in Example 13.

### Example 14. (R)-4-(6-((4-(2-(3-Chlorobenzyl)-5-methyloxazol-4-yl)phenoxy)methyl)nicotinoyl)morpholine-2-carboxylic acid (Compound 27)

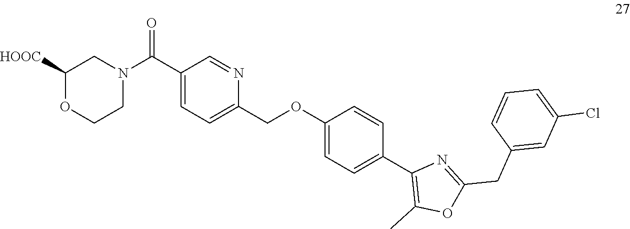 [SMILES: Cc1oc(Cc2cccc(Cl)c2)nc1-c1ccc(OCc2ccc(C(=O)N3CCO[C@@H](C(=O)O)C3)cn2)cc1]

### Step A. 2,5-Dioxopyrrolidin-1-yl 6-((4-(2-(3-chlorobenzyl)-5-methyloxazol-4-yl)phenoxy)methyl)nicotinate (27b)

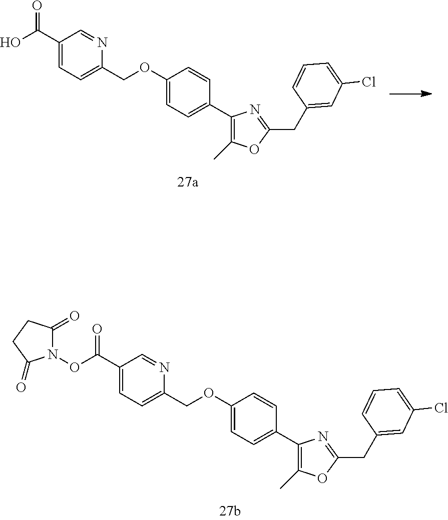 [SMILES: Cc1oc(Cc2cccc(Cl)c2)nc1-c1ccc(OCc2ccc(C(=O)O)cn2)cc1 ; Cc1oc(Cc2cccc(Cl)c2)nc1-c1ccc(OCc2ccc(C(=O)ON3C(=O)CCC3=O)cn2)cc1]

To a solution of 27a (267 mg, 0.61 mmol) and 1-hydroxypyrrolidine-2,5-dione (91.9 mg, 0.80 mmol) in DMF (3.1 mL) was added EDCI HCl at room temperature. After stirring for 3 h, additional 1-hydroxypyrrolidine-2,5-dione (36.7 mg) and EDCI (61 mg) were added and the reaction was stirred for another 20 min. The reaction was diluted with water and extracted with EtOAc (×3). The combined organic layer was washed with water, brine (×2), dried over Na₂SO₄, concentrated under reduced pressure and purified by silica gel column chromatography (gradient elution, 20-100% EtOAc in hexanes) to provide the title product (27b) (304 mg, 93%) as an off-white solid. m/z (ESI, +ve ion)=532.2 [M+H]⁺.

### Step B. (R)-4-(6-((4-(2-(3-Chlorobenzyl)-5-methyloxazol-4-yl)phenoxy)methyl)nicotinoyl)morpholine-2-carboxylic acid (27)

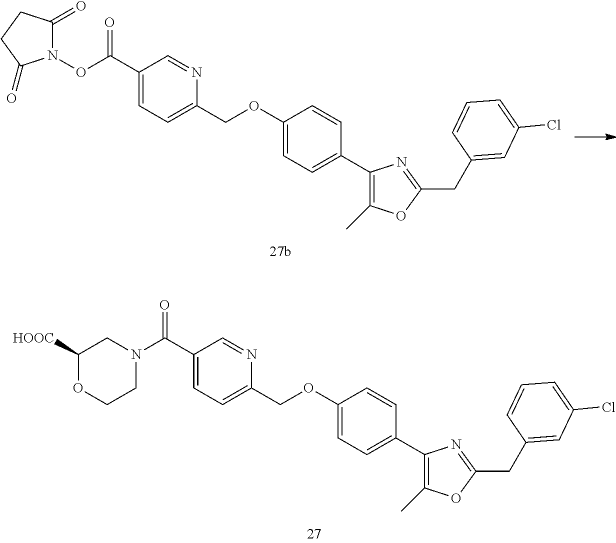 [SMILES: Cc1oc(Cc2cccc(Cl)c2)nc1-c1ccc(OCc2ccc(C(=O)ON3C(=O)CCC3=O)cn2)cc1 ; Cc1oc(Cc2cccc(Cl)c2)nc1-c1ccc(OCc2ccc(C(=O)N3CCO[C@@H](C(=O)O)C3)cn2)cc1]

To a solution of (2R)-morpholine-2-carboxylic acid hydrochloride (15.8 mg, 0.094 mmol) in DMF (0.25 mL) was added DIPEA (0.049 mL, 0.28 mmol) at room temperature and the reaction was stirred for 10 min. 27b (20 mg, 0.038 mmol) in DMF (0.25 mL) was added the resulting mixture was heated at 50° C. for 3 h. After cooling, the reaction was diluted with water and EtOAc, acidified by 1 N HCl and extracted with EtOAc (×3). The combined organic layer was washed with water, brine (×2), dried over Na₂SO₄, filtered and concentrated under reduced pressure. The crude residue was purified by reverse phase HPLC (40-80% MeCN in water, with 0.1% TFA as a modifier) to provide the title product (27) (13.9 mg, 56%) as a white solid. m/z (ESI, +ve ion)=548.3 [M+H]⁺. ¹H NMR (600 MHz, CD₃OD) δ ppm 8.69 (br s, 1H), 7.97-8.03 (m, 1H), 7.75 (d, J=8.07 Hz, 1H), 7.51-7.60 (m, 2H), 7.30-7.37 (m, 2H), 7.25-7.30 (m, 2H), 7.09-7.13 (m, 2H), 5.31 (s, 2H), 4.26 (br dd, J=8.44, 3.30 Hz, 1H) 4.12 (s, 2H), 3.94-4.08 (m, 2H), 3.69-3.83 (m, 2H), 3.51-3.64 (m, 2H), 2.45 (s, 3H).

### Example 15. (3,8-Diazabicyclo[3.2.1]octan-3-yl)(6-((4-(2-(3-chlorobenzyl)-5-methyloxazol-4-yl)phenoxy)methyl)pyridin-3-yl)methanone (Compound 29)

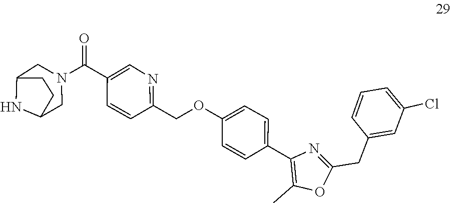 [SMILES: Cc1oc(Cc2cccc(Cl)c2)nc1-c1ccc(OCc2ccc(C(=O)N3CC4CCC(C3)N4)cn2)cc1]

### Step A. tert-Butyl 3-(6-((4-(2-(3-chlorobenzyl)-5-methyloxazol-4-yl)phenoxy)methyl)nicotinoyl)-3,8-diazabicyclo[3.2.1]octane-8-carboxylate (29)

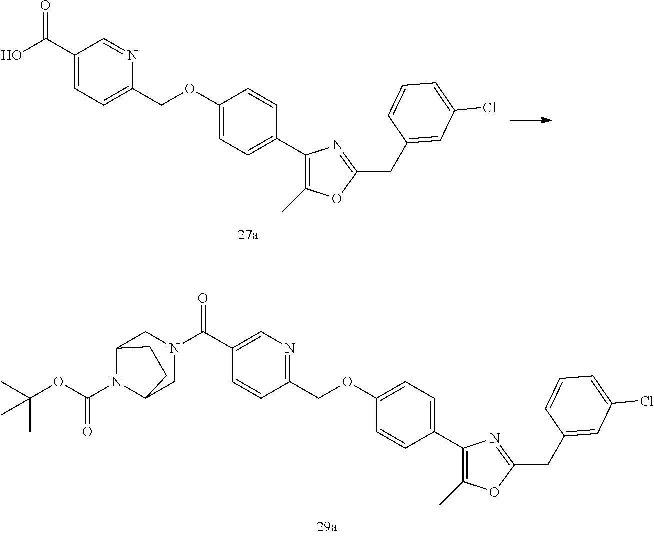 [SMILES: Cc1oc(Cc2cccc(Cl)c2)nc1-c1ccc(OCc2ccc(C(=O)O)cn2)cc1 ; Cc1oc(Cc2cccc(Cl)c2)nc1-c1ccc(OCc2ccc(C(=O)N3CC4CCC(C3)N4C(=O)OC(C)(C)C)cn2)cc1]

The title compound 29a was synthesized from 27a and tert-butyl 3,8-diazabicyclo[3.2.1]octane-8-carboxylate as described for the synthesis of Compound 22 in Example 12, Step D. m/z (ESI, +ve ion)=629.2 [M+H]⁺.

### Step B. tert-Butyl 3-(6-((4-(2-(3-chlorobenzyl)-5-methyloxazol-4-yl)phenoxy)methyl)nicotinoyl)-3,8-diazabicyclo[3.2.1]octane-8-carboxylate (29)

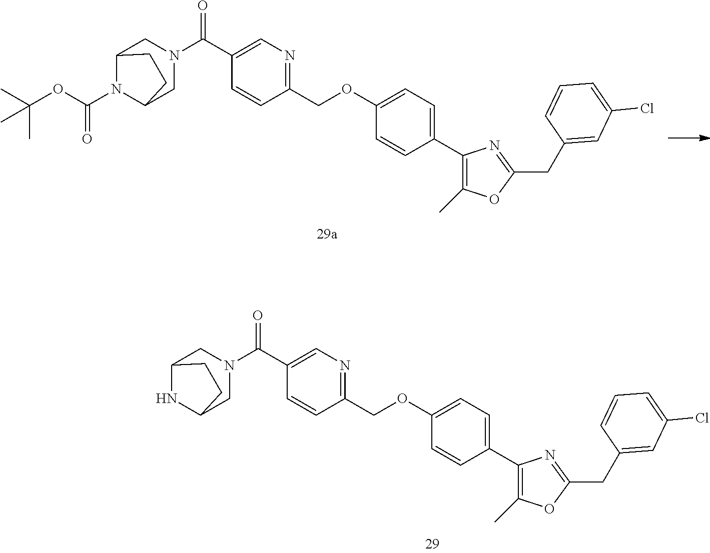 [SMILES: Cc1oc(Cc2cccc(Cl)c2)nc1-c1ccc(OCc2ccc(C(=O)N3CC4CCC(C3)N4C(=O)OC(C)(C)C)cn2)cc1 ; Cc1oc(Cc2cccc(Cl)c2)nc1-c1ccc(OCc2ccc(C(=O)N3CC4CCC(C3)N4)cn2)cc1]

To a stirred solution of 29a (24.5 mg, 0.039 mmol) in DCM was added 4 N HCl in 1,4-dioxane at room temperature. After 2 h, product was formed as a sticky oil at the bottom of reaction flask. The supernatant was decanted off. The oil was diluted with DCM and sonicated. The supernatant was removed and remaining residue was lyophilized to provide the title product (29) (18.3 mg, 83%) as a white solid. m/z (ESI, +ve ion)=529.3 [M+H]⁺. ¹H NMR (600 MHz, CD₃OD) δ ppm 8.75 (d, J=1.83 Hz, 1H), 8.09 (dd, J=8.07, 2.20 Hz, 1H), 7.82 (d, J=8.07 Hz, 1H), 7.55-7.58 (m, 2H), 7.23-7.38 (m, 4H), 7.11-7.14 (m, 2H), 5.34 (s, 2H), 4.53-4.76 (m, 1H), 3.94-4.32 (m, 5H), 3.59-3.85 (m, 2H), 2.46 (s, 3H), 1.85-2.20 (m, 4H).

### Example 16. 1-(4-(Benzyloxy)phenyl)-1-oxopropan-2-yl 2-(1-methyl-1H-pyrazol-4-yl)acetate (Compound 30)

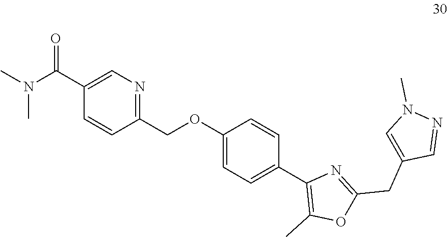 [SMILES: Cc1oc(Cc2cnn(C)c2)nc1-c1ccc(OCc2ccc(C(=O)N(C)C)cn2)cc1]

### Step A. 1-(4-(Benzyloxy)phenyl)-1-oxopropan-2-yl 2-(1-methyl-1H-pyrazol-4-yl)acetate (30a)

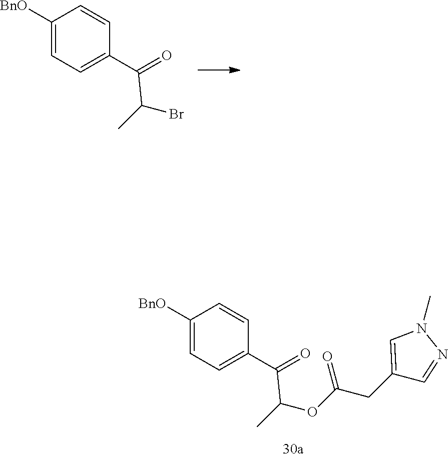 [SMILES: CC(Br)C(=O)c1ccc(OCc2ccccc2)cc1 ; CC(OC(=O)Cc1cnn(C)c1)C(=O)c1ccc(OCc2ccccc2)cc1]

A mixture of 1-(4-(benzyloxy)phenyl)-2-bromopropan-1-one (760 mg, 2.4 mmol), 2-(1-methyl-1H-pyrazol-4-yl)acetic acid (334 mg, 2.4 mmol) and Cs₂CO₃ (470 mg, 1.4 mmol) in DMF (10 mL) was stirred at 20° C. for 4 h. After completion, the reaction was diluted with water (50 mL) and extracted with EtOAc (50 mL×3). The combined organic layer was washed with water (20 mL), brine (20 mL), dried with anhydrous sodium sulfate, filtered and concentrated to afford the crude title product (30a) (1.2 g) as a yellow oil, which was used in the next step without further purification. m/z (ESI, +ve ion)=379.2 [M+H]⁺.

### Step B. 1-(4-Hydroxyphenyl)-1-oxopropan-2-yl 2-(1-methyl-1H-pyrazol-4-yl)acetate (30b)

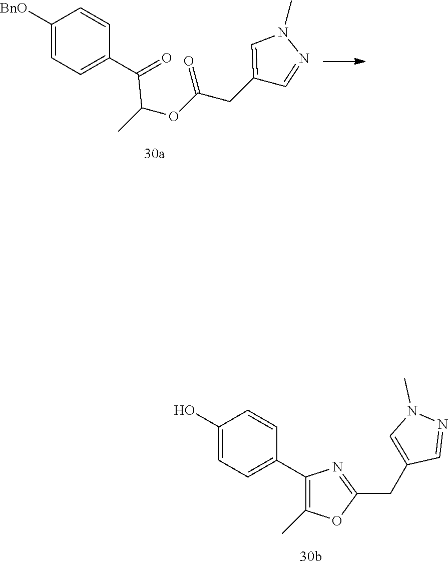 [SMILES: CC(OC(=O)Cc1cnn(C)c1)C(=O)c1ccc(OCc2ccccc2)cc1 ; Cc1oc(Cc2cnn(C)c2)nc1-c1ccc(O)cc1]

The title compound was synthesized in similar procedures as described for the synthesis of Compound 21 in Example 11, Step F and G. m/z (ESI, +ve ion)=270.1 [M+H]⁺.

### Step C. Methyl 6-((4-(5-methyl-2-((1-methyl-1H-pyrazol-4-yl)methyl)oxazol-4-yl)phenoxy)methyl)nicotinate (30c)

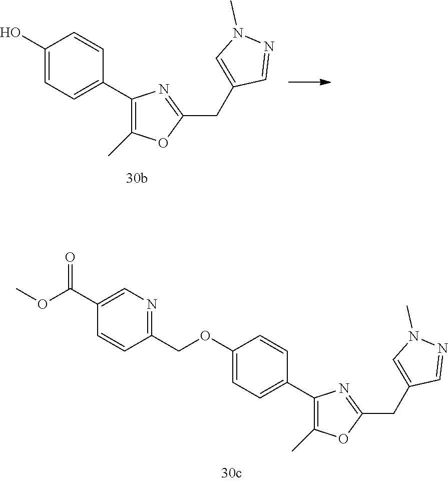 [SMILES: Cc1oc(Cc2cnn(C)c2)nc1-c1ccc(O)cc1 ; COC(=O)c1ccc(COc2ccc(-c3nc(Cc4cnn(C)c4)oc3C)cc2)nc1]

The title compound (30c) was synthesized from 30b in a similar procedure as described for the synthesis of Compound 22 in Example 12, Step B. m/z (ESI, +ve ion)=419.1 [M+H]⁺.

### Step D. 1-(4-(Benzyloxy)phenyl)-1-oxopropan-2-yl 2-(1-methyl-1H-pyrazol-4-yl)acetate (30)

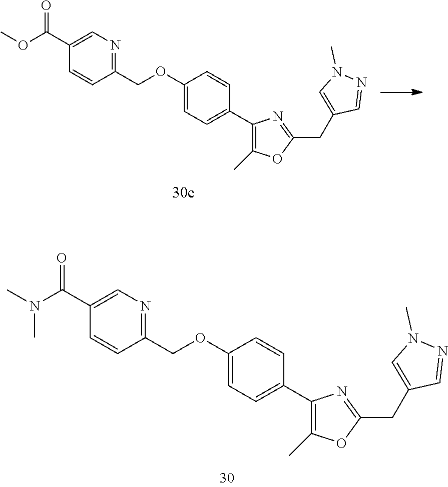 [SMILES: COC(=O)c1ccc(COc2ccc(-c3nc(Cc4cnn(C)c4)oc3C)cc2)nc1 ; Cc1oc(Cc2cnn(C)c2)nc1-c1ccc(OCc2ccc(C(=O)N(C)C)cn2)cc1]

The title compound (30) was synthesized from 30c in similar procedures as described for the synthesis of Compound 23 in Example 13. m/z (ESI, +ve ion)=432.2 [M+H]. ¹H NMR (400 MHz, CD₃OD) δ ppm 8.64 (d, J=2.19 Hz, 1H), 7.94 (dd, J=8.11, 1.97 Hz, 1H), 7.70 (d, J=7.89 Hz, 1H), 7.49-7.60 (m, 3H), 7.42 (s, 1H), 7.05-7.12 (m, 2H), 5.27 (s, 2H), 3.97 (s, 2H), 3.85 (s, 3H), 3.13 (s, 3H), 3.04 (s, 3H), 2.45 (s, 3H).

Compound 31 was synthesized using similar procedures for the synthesis of Compound 30 as described in Example 16, except the benzyl group was removed by heating in TFA at 45° C. for 1 h. m/z (ESI, +ve ion)=432.2 [M+H]⁺.

Compound 32 was synthesized using similar procedures as described for the synthesis of Compound 27 in Example 14.

### Example 17. 4-((4-(2-(3-Chlorobenzyl)-5-methyloxazol-4-yl)phenoxy)methyl)-3-((2-hydroxyethoxy)methyl)-N,N-dimethylbenzamide (Compound 33)

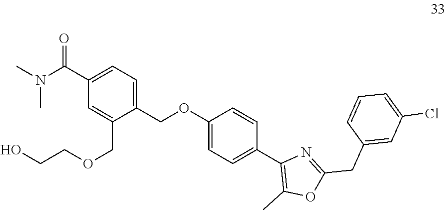 [SMILES: Cc1oc(Cc2cccc(Cl)c2)nc1-c1ccc(OCc2ccc(C(=O)N(C)C)cc2COCCO)cc1]

### Step A. Methyl 5-bromo-2-((4-(2-(3-chlorobenzyl)-5-methyloxazol-4-yl)phenoxy)methyl)benzoate (33)

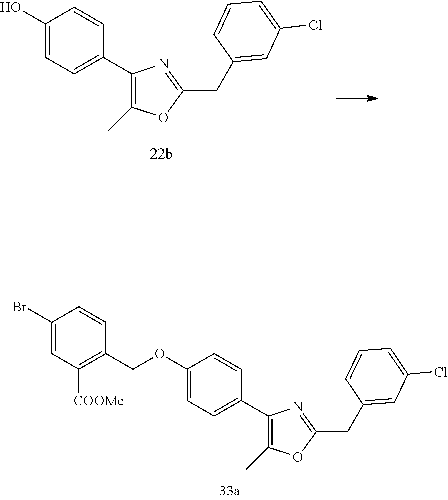 [SMILES: Cc1oc(Cc2cccc(Cl)c2)nc1-c1ccc(O)cc1 ; COC(=O)c1cc(Br)ccc1COc1ccc(-c2nc(Cc3cccc(Cl)c3)oc2C)cc1]

To a solution of methyl 5-bromo-2-(bromomethyl) benzoate (1.4 g, 4 mmol), 22b (1.35 g, 4 mmol) in DMF (10 mL) was added K₂CO₃ (1.24 g, 9 mmol) at 20° C. After stirring at 45° C. for 16 h, the reaction was diluted with EtOAc (10 mL) and H₂O (10 mL). The organic phase was separated and concentrated. The crude residue was purified by silica gel column chromatography (EtOAc: petroleum ether=1:5) to provide the title product (33a) (1.8 g, 71.1%) as an oil. m/z (ESI, +ve ion)=528.0 [M+H]⁺.

### Step B. (5-Bromo-2-((4-(2-(3-chlorobenzyl)-5-methyloxazol-4-yl)phenoxy)methyl)phenyl)methanol (33b)

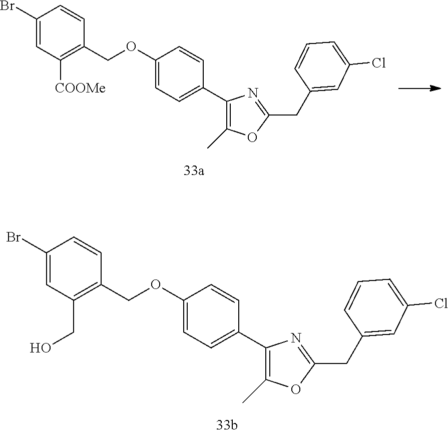 [SMILES: COC(=O)c1cc(Br)ccc1COc1ccc(-c2nc(Cc3cccc(Cl)c3)oc2C)cc1 ; Cc1oc(Cc2cccc(Cl)c2)nc1-c1ccc(OCc2ccc(Br)cc2CO)cc1]

To a solution of 33a (650 mg, 1.2 mmol) in THF (2 mL) was added lithium aluminum hydride (23.4 mg, 0.6 mmol) at 0° C. The mixture was stirred at 20° C. for 1 h. After completion, the reaction was quenched with H₂O (5 mL) at 0° C. The mixture was filtered and the filtrate was extracted with EtOAc (5 mL×3). The combined organic layer was dried, filtered, concentrated and purified by reverse phase HPLC (gradient elution, 0-50% CH₃CN in H₂O) to afford the title product (33b) (450 mg, 69.5%) as a yellow oil. m/z (ESI, +ve ion)=500.0 [M+H]⁺.

### Step C. tert-Butyl 2-((5-bromo-2-((4-(2-(3-chlorobenzyl)-5-methyloxazol-4-yl)phenoxy)methyl)benzyl)oxy)acetate (33c)

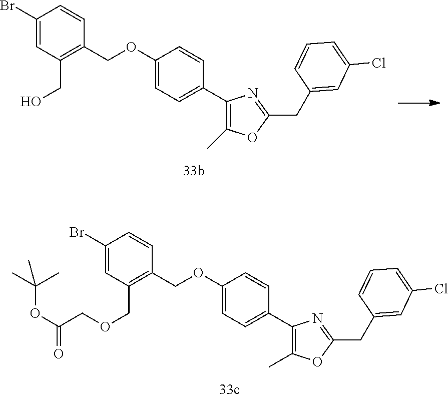 [SMILES: Cc1oc(Cc2cccc(Cl)c2)nc1-c1ccc(OCc2ccc(Br)cc2CO)cc1 ; Cc1oc(Cc2cccc(Cl)c2)nc1-c1ccc(OCc2ccc(Br)cc2COCC(=O)OC(C)(C)C)cc1]

To a solution of 33b (450 mg, 0.9 mmol) in toluene (5 mL) was added NaOH (180 mg, 50% solution), tetrabutylammonium bromide (29 mg, 0.09 mmol) and tert-butyl 2-bromoacetate (352 mg, 1.8 mmol) at 0° C. After stirring at 20° C. for 16 h, the reaction was quenched with H₂O (5 mL) and extracted with EtOAc (5 mL×3). The combined organic layer was washed with H₂O (2 mL), brine (2 mL), dried over anhydrous Na₂SO₄, filtered and concentrated in vacuo to give a crude residue, which was purified by reversed phase HPLC (gradient elution, 0-40% CH₃CN in H₂O) to afford the title product (33c) (370 mg, 63.6%) as an oil. m/z (ESI, +ve ion)=636.0 [M+Na]+.

### Step D. Methyl 3-((2-(tert-butoxy)-2-oxoethoxy)methyl)-4-((4-(2-(3-chlorobenzyl)-5-methyloxazol-4-yl)phenoxy)methyl)benzoate (33d)

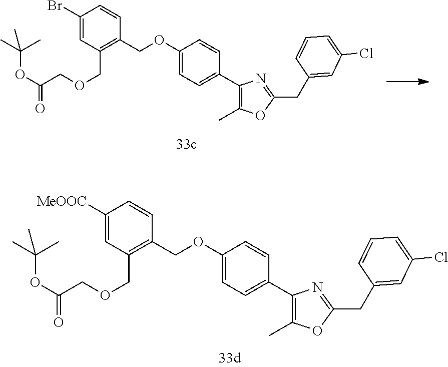 [SMILES: Cc1oc(Cc2cccc(Cl)c2)nc1-c1ccc(OCc2ccc(Br)cc2COCC(=O)OC(C)(C)C)cc1 ; COC(=O)c1ccc(COc2ccc(-c3nc(Cc4cccc(Cl)c4)oc3C)cc2)c(COCC(=O)OC(C)(C)C)c1]

To a solution of 33c (430 mg, 0.70 mmol) in MeOH/DMSO (2 mL, 1:1) was added Pd(dppf)Cl₂ (114.5 mg, 0.14 mmol) and Et₃N (800 mg, 7.0 mmol) at 20° C. The reaction was stirred under a CO atmosphere at 80° C. for 16 h. After completion, the solvent was removed and the resulting residue was added H₂O (2 mL) and extracted with EtOAc (2 mL×2). The combined organic layer was dried, filtered, concentrated and purified with prep-TLC (EtOAc:petroleum ether=1:5) to provide the title product (33d) (270 mg, 58.5%) as an oil. m/z (ESI, +ve ion)=592.3 [M+H]⁺.

### Step E. 2-((2-((4-(2-(3-Chlorobenzyl)-5-methyloxazol-4-yl)phenoxy)methyl)-5-(methoxycarbonyl)benzyl)oxy)acetic acid (33e)

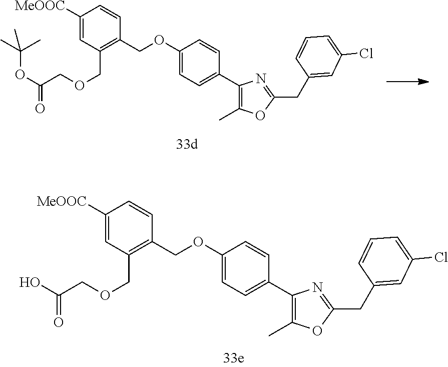 [SMILES: COC(=O)c1ccc(COc2ccc(-c3nc(Cc4cccc(Cl)c4)oc3C)cc2)c(COCC(=O)OC(C)(C)C)c1 ; COC(=O)c1ccc(COc2ccc(-c3nc(Cc4cccc(Cl)c4)oc3C)cc2)c(COCC(=O)O)c1]

To a solution of 33d (270 mg, 0.456 mmol) in DCM (2 mL) was added TFA (519 mg, 4.56 mmol) at 0° C. and the mixture was stirred for 2 h at 20° C. After completion, the mixture was evaporated to provide the crude title product (33e) as an oil (180 mg, 70%), which was used in the next step without further purification. m/z (ESI, +ve ion)=536.2 [M+H]⁺.

### Step F. Methyl 4-((4-(2-(3-chlorobenzyl)-5-methyloxazol-4-yl)phenoxy)methyl)-3-((2-hydroxyethoxy)methyl)benzoate (33f)

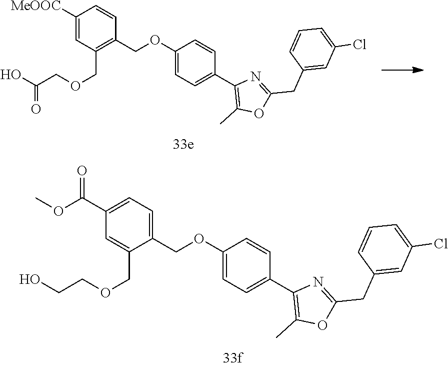 [SMILES: COC(=O)c1ccc(COc2ccc(-c3nc(Cc4cccc(Cl)c4)oc3C)cc2)c(COCC(=O)O)c1 ; COC(=O)c1ccc(COc2ccc(-c3nc(Cc4cccc(Cl)c4)oc3C)cc2)c(COCCO)c1]

To a solution of 33e (180 mg, 0.34 mmol) in THF (2 mL) was added BH₃-Me₂S (2 N in THF, 0.5 mL, 1.0 mmol) at 0° C. After heating at 40° C. for 1 h, the reaction was cooled down to 0° C. and quenched with MeOH (10 mL). The mixture was concentrated and the residue was purified with prep-TLC (EtOAc:petroleum ether=1:1) to provide the title product (33f) (120 mg, 79.2%) as an oil. m/z (ESI, +ve ion)=522.2 [M+H]⁺.

### Step G. 4-((4-(2-(3-Chlorobenzyl)-5-methyloxazol-4-yl)phenoxy)methyl)-3-((2-hydroxyethoxy)methyl)-N,N-dimethylbenzamide) (33)

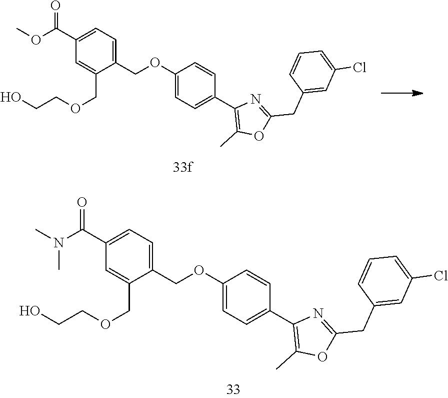 [SMILES: COC(=O)c1ccc(COc2ccc(-c3nc(Cc4cccc(Cl)c4)oc3C)cc2)c(COCCO)c1 ; Cc1oc(Cc2cccc(Cl)c2)nc1-c1ccc(OCc2ccc(C(=O)N(C)C)cc2COCCO)cc1]

To a solution of dimethylamine hydrochloride (18 mg, 0.4 mmol) in toluene (2 mL) was added trimethyl aluminum (0.2 mL, 0.4 mmol, 2 M in hexanes) at 0° C. After stirring at 20° C. for 30 min, 33f (70 mg, 0.13 mmol) was added and the mixture was heated at 100° C. for 1 h. After completion, the mixture was cooled to 0° C., and 1 N HCl (0.5 mL) was added. The reaction was concentrated and the crude residue was purified with reversed phase HPLC (gradient elution, 0-50% CH₃CN in H₂O) to provide the title product (33) (16.4 mg, 22.6%) as a white solid. m/z (ESI, +ve ion)=535.2 [M+H]⁺. ¹H NMR (400 MHz, CD₃OD) δ ppm 7.55-7.58 (m, 2H), 7.53 (d, J=8.00 Hz, 2H), 7.38 (d, J=8.00 Hz, 1H), 7.34 (s, 1H), 7.20-7.27 (m, 3H), 7.02 (d, J=8.00 Hz, 2H), 5.18 (s, 2H), 4.69 (s, 2H), 4.09 (s, 2H), 3.73-3.78 (m, 2H), 3.58-3.64 (m, 2H), 3.12 (s, 3H), 3.10 (s, 3H), 2.46 (s, 3H), 1.94 (br s, 1H).

### Example 18. (S)-(3-Aminopiperidin-1-yl)(6-((4-(2-(3-chlorobenzyl)-5-methyloxazol-4-yl)phenoxy)methyl)pyridin-3-yl)methanone (Compound 34)

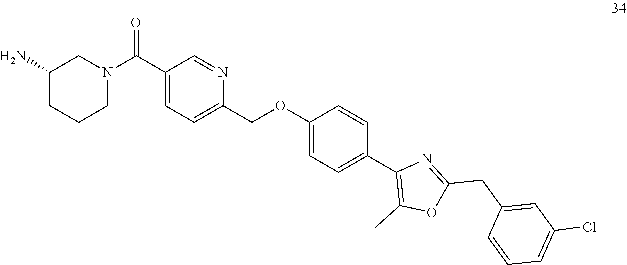 [SMILES: Cc1oc(Cc2cccc(Cl)c2)nc1-c1ccc(OCc2ccc(C(=O)N3CCC[C@H](N)C3)cn2)cc1]

### Step A. tert-Butyl (S)-(1-(6-((4-(2-(3-chlorobenzyl)-5-methyloxazol-4-yl)phenoxy)methyl)nicotinoyl)piperidin-3-yl)carbamate (34a)

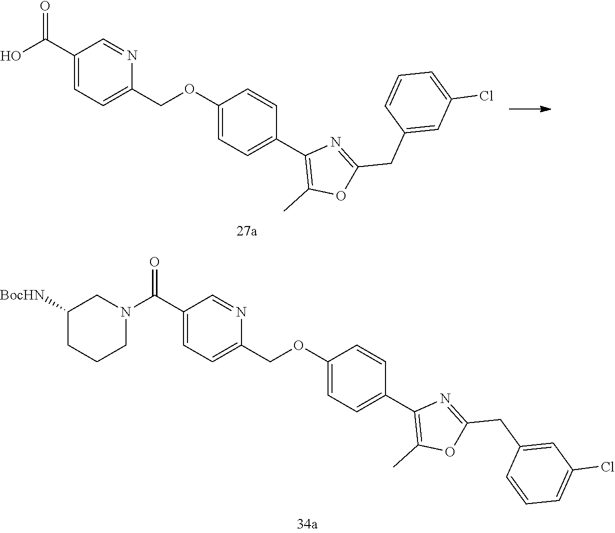 [SMILES: Cc1oc(Cc2cccc(Cl)c2)nc1-c1ccc(OCc2ccc(C(=O)O)cn2)cc1 ; Cc1oc(Cc2cccc(Cl)c2)nc1-c1ccc(OCc2ccc(C(=O)N3CCC[C@H](NC(=O)OC(C)(C)C)C3)cn2)cc1]

To a mixture of 27a (90 mg, 0.21 mmol), tert-butyl (3S)-piperidin-3-ylamino formate (125 mg, 0.62 mmol) in DCM (5 ml) was added DIPEA (80 mg, 0.62 mmol), and T₃P (172 mg, 0.27 mmol, 50% in EtOAc). The mixture was stirred for 2 h at 25° C. The reaction was quenched by adding H₂O (50 mL) and extracted with ethyl acetate (50 mL×3). The combined organic layer was dried over Na₂SO₄, filtered and concentrated under reduced pressure to afford the crude title product (34a) (110 mg), which was used in the next step without further purificaiton. m/z (ESI, +ve ion)=617.2 [M+H]⁺.

### Step B. (S)-(3-Aminopiperidin-1-yl)(6-((4-(2-(3-chlorobenzyl)-5-methyloxazol-4-yl)phenoxy)methyl)pyridin-3-yl)methanone (34)

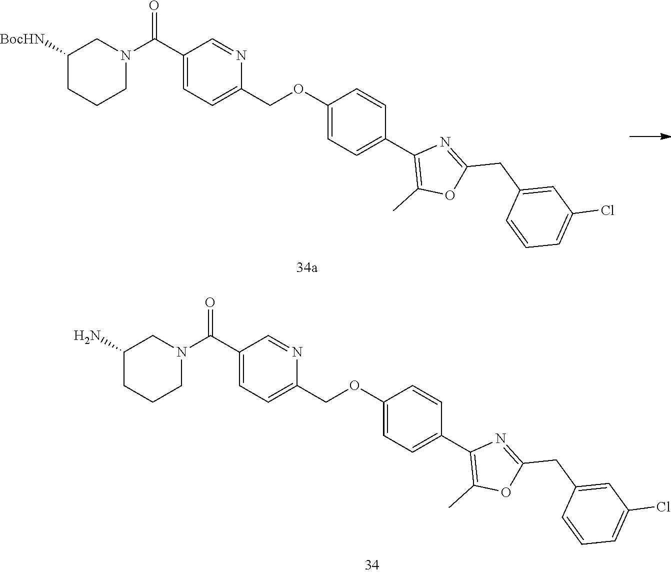 [SMILES: Cc1oc(Cc2cccc(Cl)c2)nc1-c1ccc(OCc2ccc(C(=O)N3CCC[C@H](NC(=O)OC(C)(C)C)C3)cn2)cc1 ; Cc1oc(Cc2cccc(Cl)c2)nc1-c1ccc(OCc2ccc(C(=O)N3CCC[C@H](N)C3)cn2)cc1]

To a solution of 34a (120 mg, 0.19 mmol) in DCM (4 mL) was added TFA (1 mL) at 0° C. The reaction was stirred for 2 h at 25° C. Then H₂O (50 mL) was added, and the reaction was extracted with ethyl acetate (50 mL×3). The combined organic layer was dried over Na₂SO₄, filtered, and concentrated under reduced pressure to provide a crude residue. The residue was purified by reverse phase HPLC (85% CH₃CN in H₂O, with 0.1% NH₄OH as a modifier) to afford the title product (34) (45 mg, 44.3%) as a white solid. m/z (ESI, +ve ion)=517.2 [M+H]⁺. H NMR (400 MHz, CD₃OD) δ ppm 8.63 (d, J=1.75 Hz, 1H), 7.93 (dd, J=7.89, 2.19 Hz, 1H), 7.71 (d, J=8.33 Hz, 1H), 7.56 (d, J=8.77 Hz, 2H), 7.21-7.38 (m, 4H), 7.11 (br d, J=14.47 Hz, 2H), 5.27 (s, 2H), 4.30-4.49 (m, 1H), 4.11 (s, 2H), 3.49-3.76 (m, 1H), 2.98-3.22 (m, 1H), 2.69-2.97 (m, 2H), 2.45 (s, 3H), 1.97-2.08 (m, 1H), 1.69-1.92 (m, 1H), 1.50-1.65 (m, 1H), 1.34-1.49 (m, 1H).

Compound 35, 36, 37, 38, 39, 40, 41 were synthesized using similar procedures as described for the synthesis of Compound 34 in Example 18.

### Example 19. (3S,5R)-1-Cyano-5-methylpyrrolidin-3-yl (R)-1-acetyl-4-(5-(benzyloxy)-1H-indole-2-carbonyl)piperazine-2-carboxylate (Compound 42)

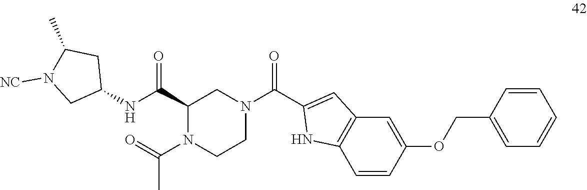 [SMILES: CC(=O)N1CCN(C(=O)c2cc3cc(OCc4ccccc4)ccc3[nH]2)C[C@@H]1C(=O)N[C@H]1C[C@@H](C)N(C#N)C1]

### Step A. 1-(tert-Butyl) 3-methyl (R)-4-acetylpiperazine-1,3-dicarboxylate (42a)

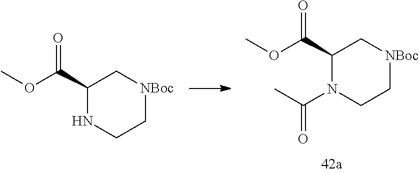 [SMILES: COC(=O)[C@H]1CN(C(=O)OC(C)(C)C)CCN1 ; COC(=O)[C@H]1CN(C(=O)OC(C)(C)C)CCN1C(C)=O]

To a solution of 1-tert-butyl 3-methyl (3R)-piperazine-1,3-dicarboxylate (4 g, 16.4 mmol) in DCM (50 mL) was added Et₃N (4.96 g, 49.1 mmol), followed acetyl chloride (1.41 g, 18 mmol) dropwise at 0° C. The reaction was stirred at 0° C. for 6 h, diluted with H₂O (10 mL) and extracted with EtOAc (20 mL×3). The combined organic layer was washed with brine (20 mL), dried over Na₂SO₄, filtered, concentrated and purified by silica gel column chromatography (DCM:MeOH=10:1) to afford the title product (42a) as a white solid (3.9 g, 77%). m/z (ESI, +ve ion)=287.1 [M+H]⁺.

### Step B. Methyl (R)-1-acetylpiperazine-2-carboxylate (42b)

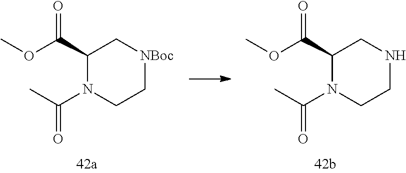 [SMILES: COC(=O)[C@H]1CN(C(=O)OC(C)(C)C)CCN1C(C)=O ; COC(=O)[C@H]1CNCCN1C(C)=O]

To a solution of 42a (2.65 g, 9 mmol) in THF (2 mL) was added 10 mL HCl (2 N in EtOAc). After stirring at 25° C. for 2 h, the reaction was concentrated to afford the crude title product (42b) (1.9 g, 91%) as a white solid, which was used in the next step without further purification. m/z (ESI, +ve ion)=187.1 [M+H]⁺.

### Step C. Methyl (R)-1-acetyl-4-(5-(benzyloxy)-1H-indole-2-carbonyl)piperazine-2-carboxylate (42c)

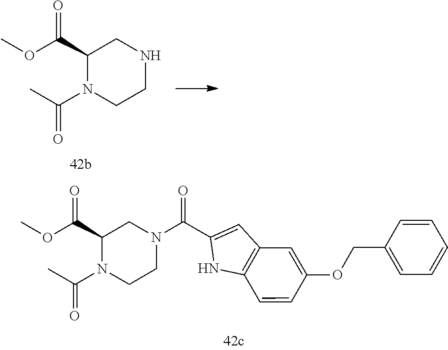 [SMILES: COC(=O)[C@H]1CNCCN1C(C)=O ; COC(=O)[C@H]1CN(C(=O)c2cc3cc(OCc4ccccc4)ccc3[nH]2)CCN1C(C)=O]

To a solution of 42b (300 mg, 1.05 mmol) in DMF (5 mL) was added Et₃N (424.2 mg, 4.2 mmol), T₃P (50% in EtOAc, 1.6 g, 4.2 mmol) and 5-(benzyloxy)-1H-indole-2-carboxylic acid (360 mg, 1.45 mmol). The mixture was stirred at 25° C. for 4 h. The reaction was diluted with H₂O (10 mL) and extracted with EtOAc (10 mL×3). The combined organic layer was dried over NaSO₄, filtered, concentrated and purified by silica gel column chromatography (DCM:MeOH=10:1) to afford the title product (42c) (250 mg, 44.6%) as a white solid. m/z (ESI, +ve ion)=436.1 [M+H]⁺.

### Step D. (R)-1-acetyl-4-(5-(benzyloxy)-1H-indole-2-carbonyl)piperazine-2-carboxylic acid (42d)

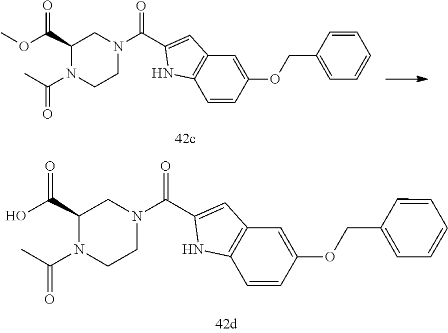 [SMILES: COC(=O)[C@H]1CN(C(=O)c2cc3cc(OCc4ccccc4)ccc3[nH]2)CCN1C(C)=O ; CC(=O)N1CCN(C(=O)c2cc3cc(OCc4ccccc4)ccc3[nH]2)C[C@@H]1C(=O)O]

To a solution of 42c (250 mg, 0.57 mmol) in THF (5 mL) and H₂O (3 mL) was added NaOH (34.5 mg, 0.86 mmol). The reaction was stirred at 25° C. for 2 h, quenched with 1 N HCl and adjusted to pH=4, and then extracted with EtOAc (5 mL×3). The combined organic layer was dried, filtered and concentrated to afford the crude title product (42d) (210 mg, 78.9%) as a white solid, which was used in the next step without further purification. m/z (ESI, +ve ion)=422.1 [M+H]⁺.

### Step E. tert-Butyl (2R,4S)-4-((R)-1-acetyl-4-(5-(benzyloxy)-1H-indole-2-carbonyl)piperazine-2-carboxamido)-2-methylpyrrolidine-1-carboxylate (42e)

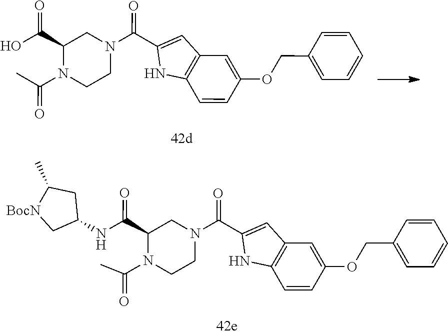 [SMILES: CC(=O)N1CCN(C(=O)c2cc3cc(OCc4ccccc4)ccc3[nH]2)C[C@@H]1C(=O)O ; CC(=O)N1CCN(C(=O)c2cc3cc(OCc4ccccc4)ccc3[nH]2)C[C@@H]1C(=O)N[C@H]1C[C@@H](C)N(C(=O)OC(C)(C)C)C1]

To a solution of 42d (120 mg, 0.28 mmol) in DCM (2 mL) was added Et₃N (84.84 mg, 0.84 mmol), HATU (319.2 mg, 0.84 mmol) and tert-butyl (2R,4S)-4-amino-2-methylpyrrolidine-1-carboxylate (56.08 mg, 0.28 mmol). The reaction was stirred at 25° C. for 4 h, diluted with DCM (5 mL×2), and washed with H₂O (5 mL×2). The combined organic layer was dried, filtered, concentrated and purified by reverse phase HPLC (CH₃CN in H₂O) to afford the title product (42e) (50 mg, 34%) as a white solid. m/z (ESI, +ve ion)=504.2 [M-Boc+H]⁺.

### Step F. (R)-1-Acetyl-4-(5-(benzyloxy)-1H-indole-2-carbonyl)-N-((3S,5R)-5-methylpyrrolidin-3-yl)piperazine-2-carboxamide (42f)

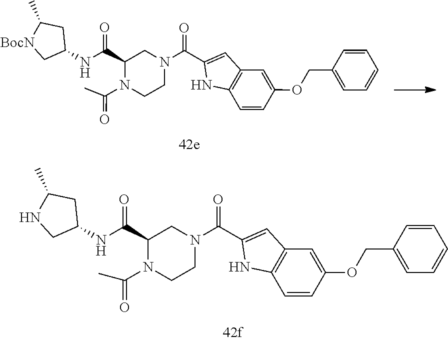 [SMILES: CC(=O)N1CCN(C(=O)c2cc3cc(OCc4ccccc4)ccc3[nH]2)C[C@@H]1C(=O)N[C@H]1C[C@@H](C)N(C(=O)OC(C)(C)C)C1 ; CC(=O)N1CCN(C(=O)c2cc3cc(OCc4ccccc4)ccc3[nH]2)C[C@@H]1C(=O)N[C@@H]1CN[C@H](C)C1]

To a solution of 42e (50 mg, 0.08 mmol) in THF (2 mL) was added HCl (2 N in EtOAc, 0.1 mL). After stirring at 25° C. for 2 h, the reaction was concentrated and purified by reverse phase HPLC (CH₃CN in H₂O) to afford the title product (42f) as a white solid (40 mg, 95%). m/z (ESI, +ve ion)=504.2 [M+H]⁺.

### Step G. (3S,5R)-1-Cyano-5-methylpyrrolidin-3-yl (R)-1-acetyl-4-(5-(benzyloxy)-1H-indole-2-carbonyl)piperazine-2-carboxylate_(42)

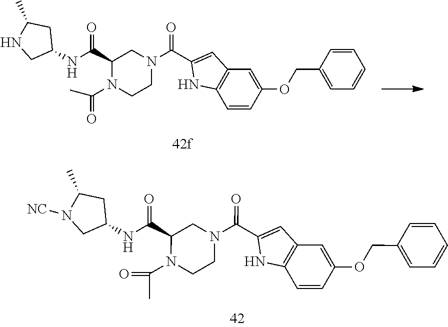 [SMILES: CC(=O)N1CCN(C(=O)c2cc3cc(OCc4ccccc4)ccc3[nH]2)C[C@@H]1C(=O)N[C@@H]1CN[C@H](C)C1 ; CC(=O)N1CCN(C(=O)c2cc3cc(OCc4ccccc4)ccc3[nH]2)C[C@@H]1C(=O)N[C@H]1C[C@@H](C)N(C#N)C1]

To a solution of 42f (30 mg, 0.06 mmol) in DCM (0.5 mL) was added DIPEA (39 mg, 0.3 mmol) and BrCN (7 mg, 0.066 mmol). After stirring at 25° C. for 2 h, the reaction was diluted with H₂O (5 mL) and extracted with DCM (5 mL×3). The combined organic phase was dried, filtered, concentrated and purified by prep-TLC (DCM:MeOH=20:1) to afford the title product (42) (8 mg, 24.2%) as a white solid. m/z (ESI, +ve ion)=529.2 [M+H]⁺.

### Example 20. (4-((1H-Imidazol-2-yl)methyl)piperidin-1-yl)(4-((4-(2-(3-chlorobenzyl)-5-methyloxazol-4-yl)benzyl)oxy)phenyl)methanone (Compound 43)

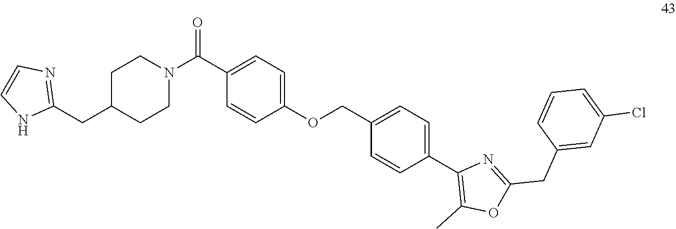 [SMILES: Cc1oc(Cc2cccc(Cl)c2)nc1-c1ccc(COc2ccc(C(=O)N3CCC(Cc4ncc[nH]4)CC3)cc2)cc1]

### Step A. Methyl 4-((4-(2-(3-chlorobenzyl)-5-methyloxazol-4-yl)benzyl)oxy)benzoate (43a)

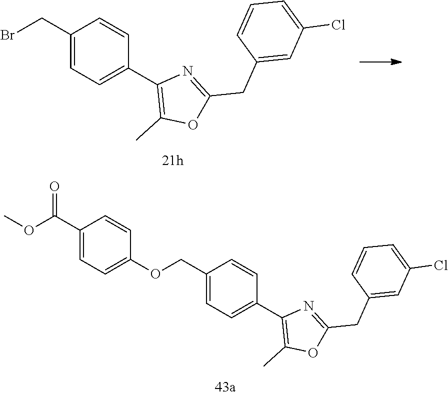 [SMILES: Cc1oc(Cc2cccc(Cl)c2)nc1-c1ccc(CBr)cc1 ; COC(=O)c1ccc(OCc2ccc(-c3nc(Cc4cccc(Cl)c4)oc3C)cc2)cc1]

A solution of 21h (70 mg, 0.19 mmol), methyl 4-hydroxybenzoate (35 mg, 0.23 mmol) and potassium carbonate (53 mg, 0.38 mmol) in DMF (2 mL) was stirred at 55° C. for 1 h. After completion, the reaction was diluted with H₂O (15 mL) and extracted with EtOAc (20 mL×3). The combined organic layer was dried over Na₂SO₄, filtered, concentrated and purified by silica gel column chromatography (EtOAc:petroleum ether=1:5) to afford the title product (43a) (78 mg, 87%) as a white solid. m/z (ESI, +ve ion)=448.1 [M+H]⁺.

### Step B. 4-((4-(2-(3-Chlorobenzyl)-5-methyloxazol-4-yl)benzyl)oxy)benzoic acid (43b)

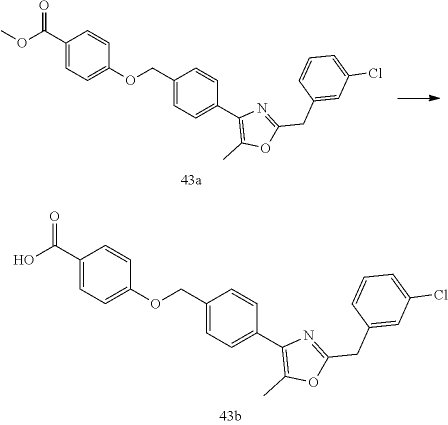 [SMILES: COC(=O)c1ccc(OCc2ccc(-c3nc(Cc4cccc(Cl)c4)oc3C)cc2)cc1 ; Cc1oc(Cc2cccc(Cl)c2)nc1-c1ccc(COc2ccc(C(=O)O)cc2)cc1]

To a stirred solution of 43a (350 mg, 3.15 mmol) in MeOH (10 mL) was added a solution of sodium hydroxide (125 mg, 12.6 mmol) in H₂O (10 mL). The reaction mixture was heated at 50° C. for 2 h. After completion, 1 N HCl was added to the reaction to adjust to pH=5 in an ice bath and the reaction was extracted with EtOAc (5 mL×3). The combined organic layer was dried over Na₂SO₄, filtered, concentrated and purified by silica gel column chromatography (gradient elution, 20-30% EtOAc in petroleum ether) to afford the title product (43b) (280 mg, 93%) as a white solid. m/z (ESI, +ve ion)=434.1 [M+H]⁺.

### Step C. 2,5-Dioxopyrrolidin-1-yl 4-((4-(2-(3-chlorobenzyl)-5-methyloxazol-4-yl)benzyl)oxy)benzoate (43c)

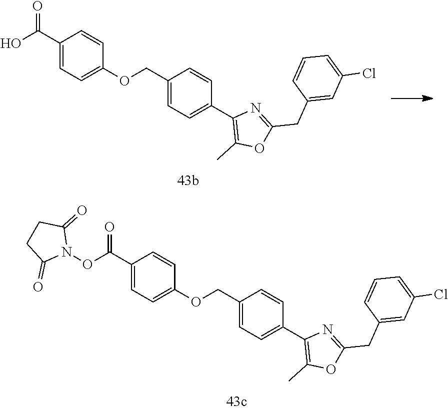 [SMILES: Cc1oc(Cc2cccc(Cl)c2)nc1-c1ccc(COc2ccc(C(=O)O)cc2)cc1 ; Cc1oc(Cc2cccc(Cl)c2)nc1-c1ccc(COc2ccc(C(=O)ON3C(=O)CCC3=O)cc2)cc1]

A solution of 43b (280 mg, 0.655 mmol), 1-hydroxypyrrolidine-2,5-dione (96 mg, 1.852 mmol) and DCC (173.2 mg, 0.84 mmol) in DCM (10 mL) was stirred at 20° C. for 2 h. After completion, the reaction was diluted with H₂O (10 mL) and extracted with EtOAc (20 mL×3). The combined organic layer was dried over Na₂SO₄, filtered, concentrated and purified by silica gel column (EtOAc:petroleum ether=1:6) to afford the title product (43c) (160 mg, 70%) as a white solid. m/z (ESI, +ve ion)=531.1 [M+H]⁺.

### Step D. (4-((1H-Imidazol-2-yl)methyl)piperidin-1-yl)(4-((4-(2-(3-chlorobenzyl)-5-methyloxazol-4-yl)benzyl)oxy)phenyl)methanone (43)

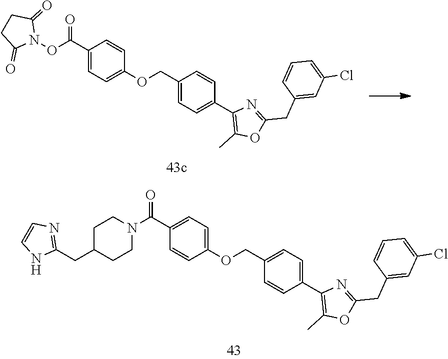 [SMILES: Cc1oc(Cc2cccc(Cl)c2)nc1-c1ccc(COc2ccc(C(=O)ON3C(=O)CCC3=O)cc2)cc1 ; Cc1oc(Cc2cccc(Cl)c2)nc1-c1ccc(COc2ccc(C(=O)N3CCC(Cc4ncc[nH]4)CC3)cc2)cc1]

To a stirred solution of 4-[(1H-imidazol-2-yl)methyl]piperidine dihydrochloride (8.1 mg, 0.034 mmol) in DMF (0.15 mL) was added DIPEA (0.015 mL, 0.085 mmol). After 10 min, 43c (15 mg, 0.028 mmol) in DMF (0.15 mL) was added. After stirring for 6 h, additional 4-[(1H-imidazol-2-yl)methyl]piperidine dihydrochloride (4 mg) and DIPEA (0.008 mL) were added and the mixture was heated at 40° C. overnight. The reaction was diluted with water, extracted with EtOAc (×3), and washed with water and brine (×2). The combined organic layer was dried over Na₂SO₄, filtered, concentrated under reduced pressure and purified by silica gel column chromatography (gradient elution, 0-15% MeOH in DCM) to provide the title product (43) (5 mg, 30%) as a white solid. m/z (ESI, +ve ion)=581.3 [M+H]⁺. ¹H NMR (600 MHz, CD₃OD) δ ppm 7.58-7.70 (m, 2H), 7.52 (d, J=8.44 Hz, 2H), 7.30-7.40 (m, 4H), 7.25-7.30 (m, 2H), 7.03-7.11 (m, 2H), 6.92 (s, 2H), 5.17 (s, 2H), 4.51-4.64 (m, 1H), 4.13 (s, 2H), 3.75-3.89 (m, 1H), 3.03-3.16 (m, 1H), 2.78-2.90 (m, 1H), 2.62-2.71 (m, 2H), 2.49 (s, 3H), 1.96-2.08 (m, 1H), 1.55-1.79 (m, 2H), 1.16-1.33 (m, 2H).

Compound 44 was synthesized as described for the synthesis of Compound 43 in Example 20.

### Example 21. Ubiquitin-Rhodamine 110 Assay to Determine USP28 IC₅₀

Each assay was performed in a final volume of 8 μl in assay buffer containing 10 mM HEPES pH 7.3 ((1 M, pH 7.3 solution, (VWR J848), 100 mM NaCl (5 M, Corning 46-032-CV), 0.01% Triton X-100 (Sigma #T8787), 3.5 mM DTT (1 M, Sigma #43819) and 0.00375% BSA (10%, Calbiochem, #126609)). The assay buffer pH was adjusted to 7.5 using 1 N NaOH (JT Baker, #5000-03). Stock compounds were stored at −80° C. as a 25 mM in DMSO solution along with their respective 20 point 2 fold serial dilutions. For the dose responses, stock compound plates were allowed to come to room temperature the day of the assay. 10 nl of the serial dilution series were pre stamped into assay plates (Black, high base, medium binding, Greiner #782076) for a final top screening concentration of 125 μM (DMSO final concentration=0.5%). Enzyme (His6 USP28, BostonBiochem, #E-570) concentration and incubation times were optimized for the maximal signal to background while maintaining the initial velocity conditions at a fixed substrate concentration. The final concentration of the enzyme in the assay was 150 pM. Final substrate (Ub-Rhol10, Ubiquitin-Rhodamine 110, UBPBio, #M3020) concentration was 41 nM. 2 μl of 4× enzyme was added to assay plates (pre stamped with compound) and incubated for 2 h at room temperature. 6 μl of 4× substrate was subsequently added to the assay plates and incubated for 2 h at room temperature. Fluorescence was then read on the Envision (Excitation 485 nm and Emission at 535 nm, Perkin Elmer).

Results are reported in Table 1 below. In the Table, IC₅₀ values are ++++<0.05 μM<+++<0.5 μM<++<1 μM<+. Molecular weights were calculated by standard techniques, and mass spectrometry results are reported according to the Examples above.

| | | MW | |
|---|---|---|---|
| | IC₅₀ | Calc. | [M + H]⁺ |
| Example Compound | (μM) | (g/mol) | Found |
| 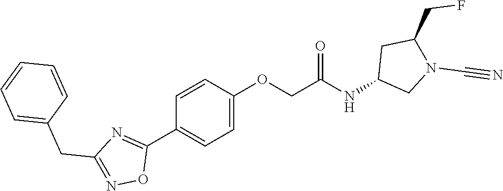 [SMILES: N#CN1C[C@H](NC(=O)COc2ccc(-c3nc(Cc4ccccc4)no3)cc2)C[C@H]1CF] | + | 435.46 | 436.2 |
| 1 | | | |
| 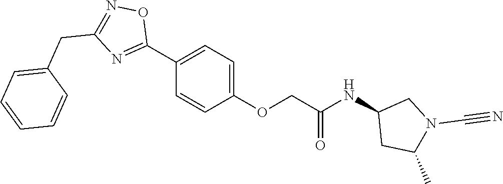 [SMILES: C[C@@H]1C[C@@H](NC(=O)COc2ccc(-c3nc(Cc4ccccc4)no3)cc2)CN1C#N] | + | 417.47 | 418.3 |
| 2 | | | |
| 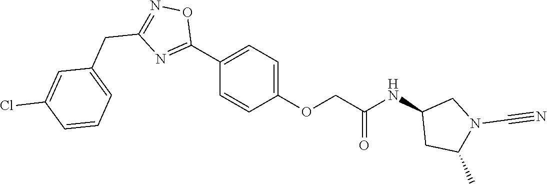 [SMILES: C[C@@H]1C[C@@H](NC(=O)COc2ccc(-c3nc(Cc4cccc(Cl)c4)no3)cc2)CN1C#N] | +++ | 451.91 | 452.2 |
| 3 | | | |
| 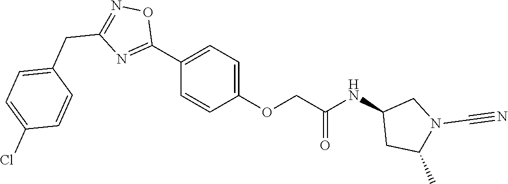 [SMILES: C[C@@H]1C[C@@H](NC(=O)COc2ccc(-c3nc(Cc4ccc(Cl)cc4)no3)cc2)CN1C#N] | +++ | 451.91 | 452.2 |
| 4 | | | |
| 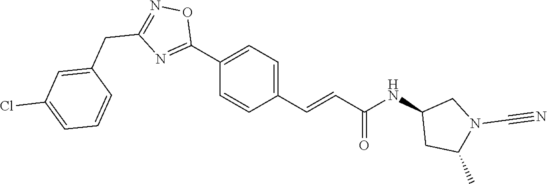 [SMILES: C[C@@H]1C[C@@H](NC(=O)/C=C/c2ccc(-c3nc(Cc4cccc(Cl)c4)no3)cc2)CN1C#N] | +++ | 447.92 | 448.2 |
| 5 | | | |
| 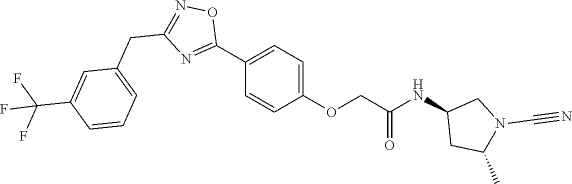 [SMILES: C[C@@H]1C[C@@H](NC(=O)COc2ccc(-c3nc(Cc4cccc(C(F)(F)F)c4)no3)cc2)CN1C#N] | +++ | 485.47 | 486.1 |
| 6 | | | |
| 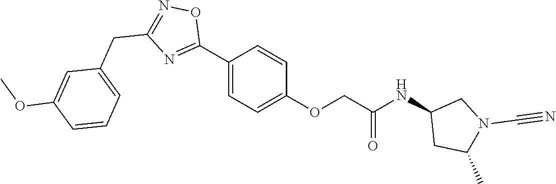 [SMILES: COc1cccc(Cc2noc(-c3ccc(OCC(=O)N[C@@H]4C[C@@H](C)N(C#N)C4)cc3)n2)c1] | + | 447.50 | 448.2 |
| 7 | | | |
| 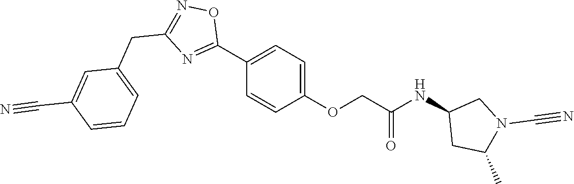 [SMILES: C[C@@H]1C[C@@H](NC(=O)COc2ccc(-c3nc(Cc4cccc(C#N)c4)no3)cc2)CN1C#N] | ++ | 442.48 | 443.3 |
| 8 | | | |
| 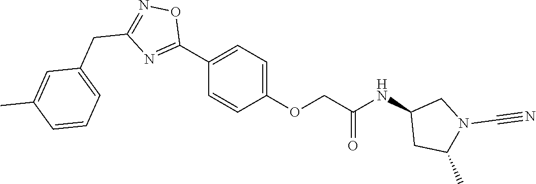 [SMILES: Cc1cccc(Cc2noc(-c3ccc(OCC(=O)N[C@@H]4C[C@@H](C)N(C#N)C4)cc3)n2)c1] | +++ | 431.496 | 432.3 |
| 9 | | | |
| 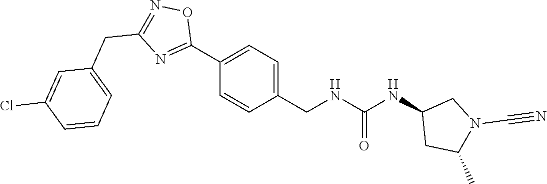 [SMILES: C[C@@H]1C[C@@H](NC(=O)NCc2ccc(-c3nc(Cc4cccc(Cl)c4)no3)cc2)CN1C#N] | +++ | 450.93 | 451.2 |
| 10 | | | |
| 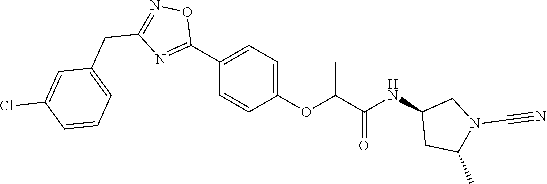 [SMILES: CC(Oc1ccc(-c2nc(Cc3cccc(Cl)c3)no2)cc1)C(=O)N[C@@H]1C[C@@H](C)N(C#N)C1] | + | 465.94 | 466.2 |
| 11 | | | |
| 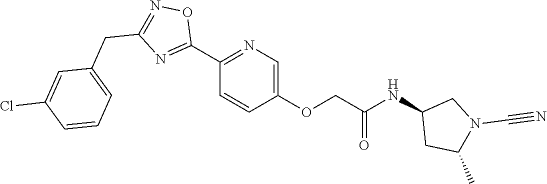 [SMILES: C[C@@H]1C[C@@H](NC(=O)COc2ccc(-c3nc(Cc4cccc(Cl)c4)no3)nc2)CN1C#N] | + | 452.9 | 453.3 |
| 12 | | | |
| 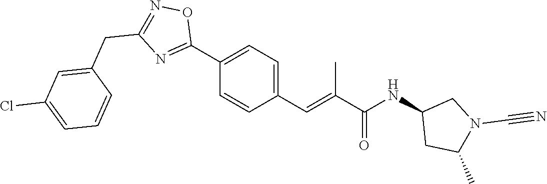 [SMILES: C/C(=C\c1ccc(-c2nc(Cc3cccc(Cl)c3)no2)cc1)C(=O)N[C@@H]1C[C@@H](C)N(C#N)C1] | ++ | 461.95 | 462.3 |
| 13 | | | |
| 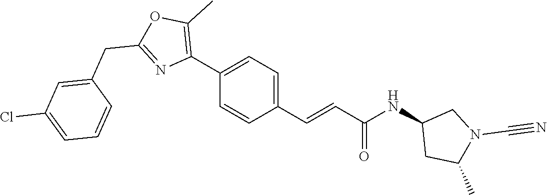 [SMILES: Cc1oc(Cc2cccc(Cl)c2)nc1-c1ccc(/C=C/C(=O)N[C@@H]2C[C@@H](C)N(C#N)C2)cc1] | +++ | 460.96 | 461.3 |
| 14 | | | |
| 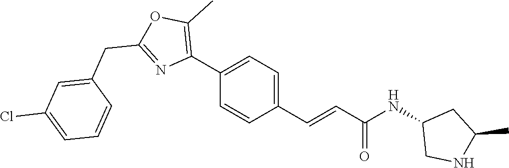 [SMILES: Cc1oc(Cc2cccc(Cl)c2)nc1-c1ccc(/C=C/C(=O)N[C@H]2CN[C@H](C)C2)cc1] | +++ | 435.95 | 436.2 |
| 15 | | | |
| 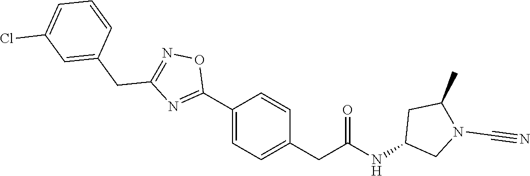 [SMILES: C[C@@H]1C[C@@H](NC(=O)Cc2ccc(-c3nc(Cc4cccc(Cl)c4)no3)cc2)CN1C#N] | + | 435.91 | 436.1 |
| 16 | | | |
| 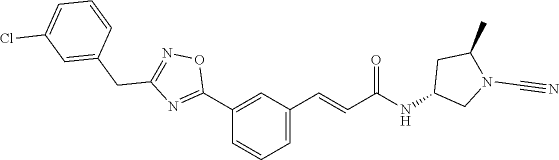 [SMILES: C[C@@H]1C[C@@H](NC(=O)/C=C/c2cccc(-c3nc(Cc4cccc(Cl)c4)no3)c2)CN1C#N] | + | 447.92 | 448.1 |
| 17 | | | |
| 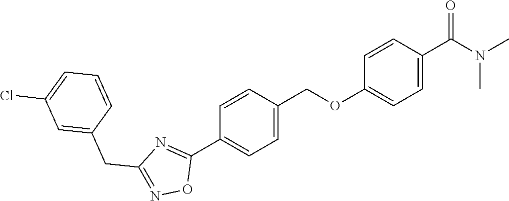 [SMILES: CN(C)C(=O)c1ccc(OCc2ccc(-c3nc(Cc4cccc(Cl)c4)no3)cc2)cc1] | +++ | 447.92 | 448.1 |
| 18 | | | |
| 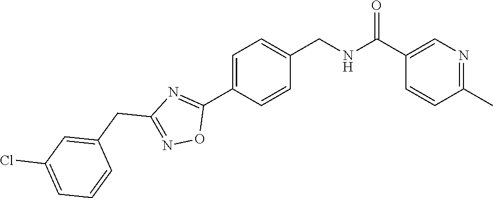 [SMILES: Cc1ccc(C(=O)NCc2ccc(-c3nc(Cc4cccc(Cl)c4)no3)cc2)cn1] | ++ | 418.88 | 419.1 |
| 19 | | | |
| 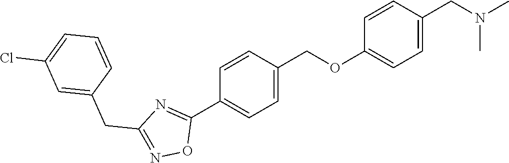 [SMILES: CN(C)Cc1ccc(OCc2ccc(-c3nc(Cc4cccc(Cl)c4)no3)cc2)cc1] | +++++ | 433.94 | 434.2 |
| 20 | | | |
| 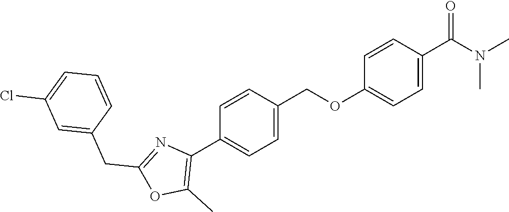 [SMILES: Cc1oc(Cc2cccc(Cl)c2)nc1-c1ccc(COc2ccc(C(=O)N(C)C)cc2)cc1] | ++++ | 460.96 | 461.3 |
| 21 | | | |
| 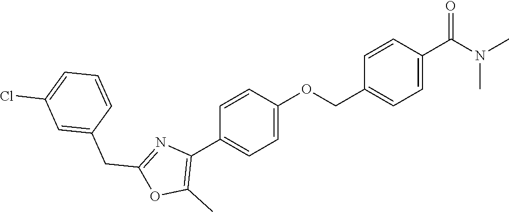 [SMILES: Cc1oc(Cc2cccc(Cl)c2)nc1-c1ccc(OCc2ccc(C(=O)N(C)C)cc2)cc1] | ++++ | 460.96 | 461.2 |
| 22 | | | |
| 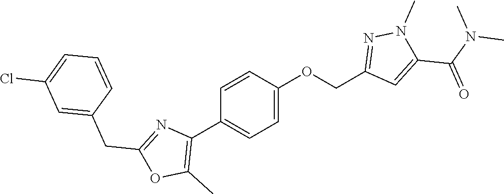 [SMILES: Cc1oc(Cc2cccc(Cl)c2)nc1-c1ccc(OCc2cc(C(=O)N(C)C)n(C)n2)cc1] | +++ | 464.95 | 465.1 |
| 23 | | | |
| 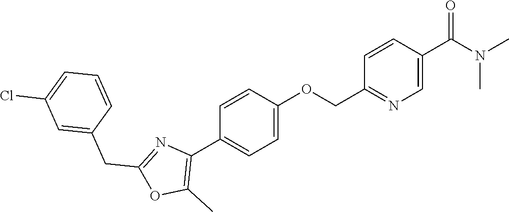 [SMILES: Cc1oc(Cc2cccc(Cl)c2)nc1-c1ccc(OCc2ccc(C(=O)N(C)C)cn2)cc1] | ++++ | 461.95 | 462.1 |
| 24 | | | |
| 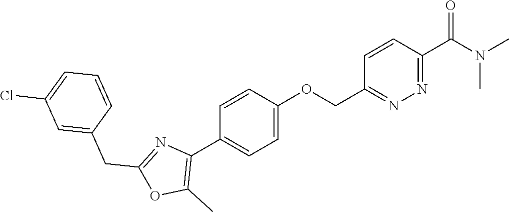 [SMILES: Cc1oc(Cc2cccc(Cl)c2)nc1-c1ccc(OCc2ccc(C(=O)N(C)C)nn2)cc1] | ++++ | 462.93 | 463.4 |
| 25 | | | |
| 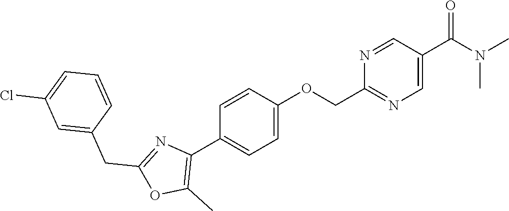 [SMILES: Cc1oc(Cc2cccc(Cl)c2)nc1-c1ccc(OCc2ncc(C(=O)N(C)C)cn2)cc1] | ++++ | 462.93 | 463.4 |
| 26 | | | |
| 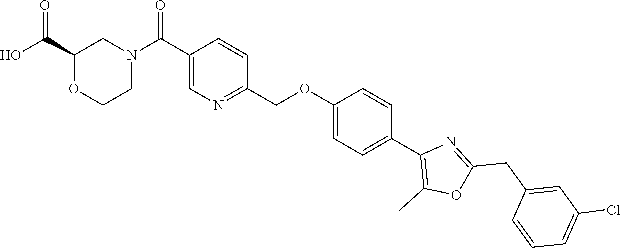 [SMILES: Cc1oc(Cc2cccc(Cl)c2)nc1-c1ccc(OCc2ccc(C(=O)N3CCO[C@@H](C(=O)O)C3)cn2)cc1] | ++++ | 547.99 | 548.3 |
| 27 | | | |
| 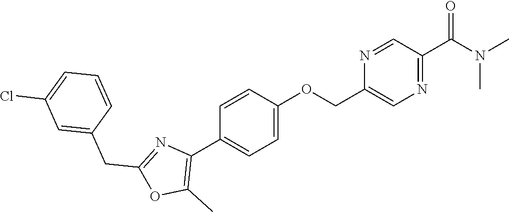 [SMILES: Cc1oc(Cc2cccc(Cl)c2)nc1-c1ccc(OCc2cnc(C(=O)N(C)C)cn2)cc1] | ++++ | 462.93 | 463.1 |
| 28 | | | |
| 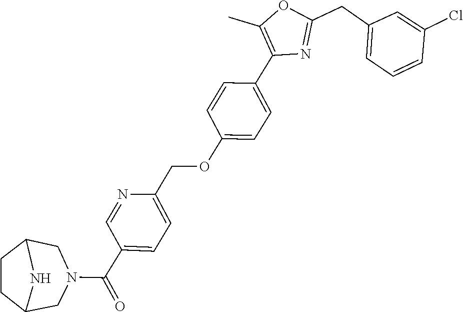 [SMILES: Cc1oc(Cc2cccc(Cl)c2)nc1-c1ccc(OCc2ccc(C(=O)N3CC4CCC(C3)N4)cn2)cc1] | ++++ | 529.04 | 529.3 |
| 29 | | | |
| 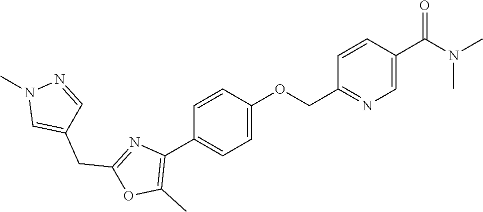 | +++ | 431.50 | 432.2 |
| 30 | | | |
| 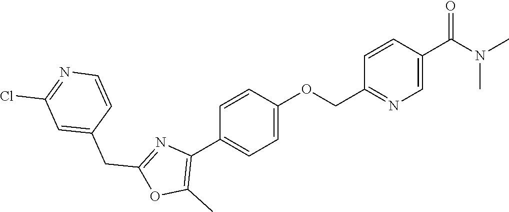 [SMILES: Cc1oc(Cc2ccnc(Cl)c2)nc1-c1ccc(OCc2ccc(C(=O)N(C)C)cn2)cc1] | +++ | 462.93 | 463.1 |
| 31 | | | |
| 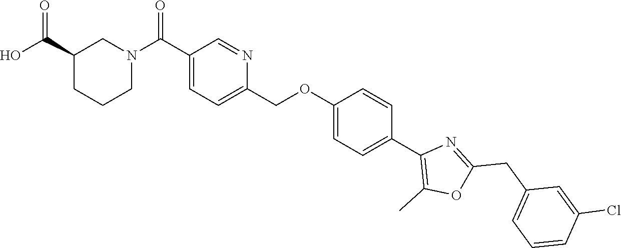 [SMILES: Cc1oc(Cc2cccc(Cl)c2)nc1-c1ccc(OCc2ccc(C(=O)N3CCC[C@@H](C(=O)O)C3)cn2)cc1] | ++++ | 546.02 | 546.3 |
| 32 | | | |
| 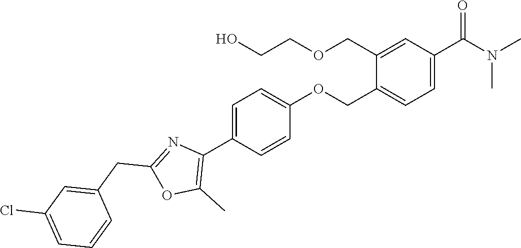 [SMILES: Cc1oc(Cc2cccc(Cl)c2)nc1-c1ccc(OCc2ccc(C(=O)N(C)C)cc2COCCO)cc1] | ++++ | 535.04 | 535.2 |
| 33 | | | |
| 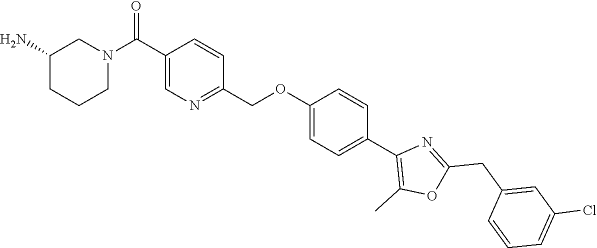 [SMILES: Cc1oc(Cc2cccc(Cl)c2)nc1-c1ccc(OCc2ccc(C(=O)N3CCC[C@H](N)C3)cn2)cc1] | ++++ | 517.03 | 517.2 |
| 34 | | | |
| 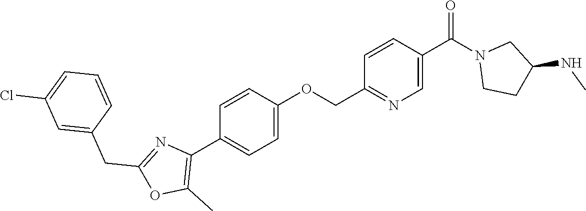 [SMILES: CN[C@H]1CCN(C(=O)c2ccc(COc3ccc(-c4nc(Cc5cccc(Cl)c5)oc4C)cc3)nc2)C1] | ++++ | 517.03 | 517.2 |
| 35 | | | |
| 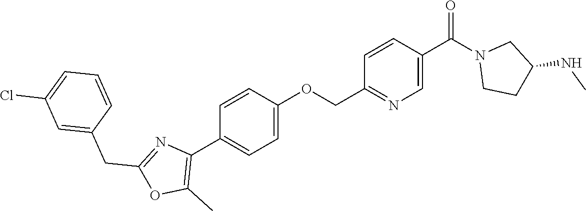 [SMILES: CN[C@@H]1CCN(C(=O)c2ccc(COc3ccc(-c4nc(Cc5cccc(Cl)c5)oc4C)cc3)nc2)C1] | ++++ | 517.03 | 517.2 |
| 36 | | | |
| 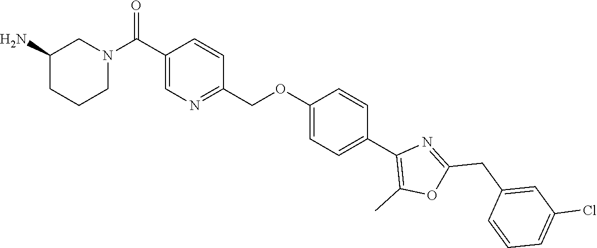 [SMILES: Cc1oc(Cc2cccc(Cl)c2)nc1-c1ccc(OCc2ccc(C(=O)N3CCC[C@@H](N)C3)cn2)cc1] | ++++ | 517.03 | 517.2 |
| 37 | | | |
| 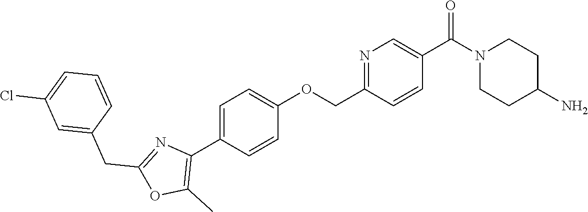 [SMILES: Cc1oc(Cc2cccc(Cl)c2)nc1-c1ccc(OCc2ccc(C(=O)N3CCC(N)CC3)cn2)cc1] | ++++ | 517.03 | 517.2 |
| 38 | | | |
| 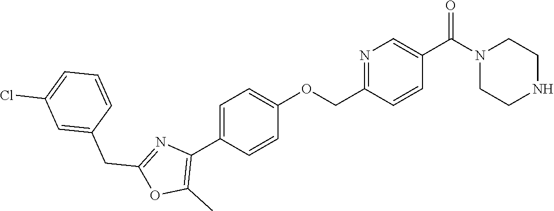 [SMILES: Cc1oc(Cc2cccc(Cl)c2)nc1-c1ccc(OCc2ccc(C(=O)N3CCNCC3)cn2)cc1] | ++++ | 503 | 503.1 |
| 39 | | | |
| 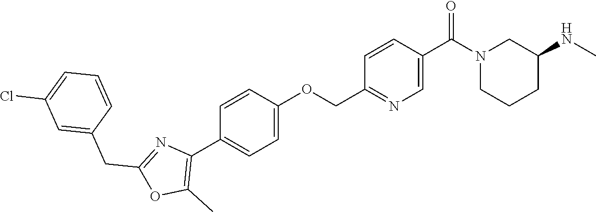 [SMILES: CN[C@H]1CCCN(C(=O)c2ccc(COc3ccc(-c4nc(Cc5cccc(Cl)c5)oc4C)cc3)nc2)C1] | ++++ | 531.05 | 531.2 |
| 40 | | | |
| 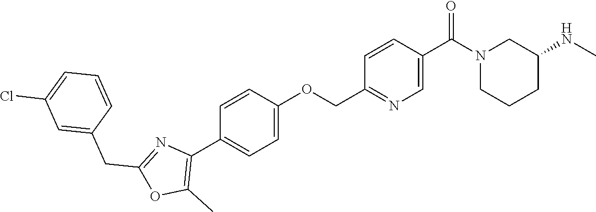 [SMILES: CN[C@@H]1CCCN(C(=O)c2ccc(COc3ccc(-c4nc(Cc5cccc(Cl)c5)oc4C)cc3)nc2)C1] | ++++ | 531.05 | 531.2 |
| 41 | | | |
| 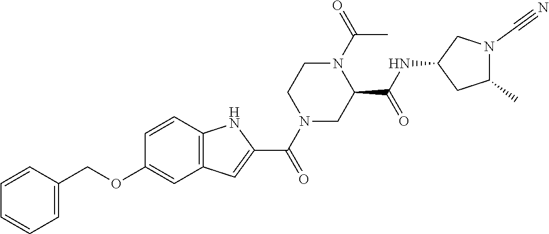 [SMILES: CC(=O)N1CCN(C(=O)c2cc3cc(OCc4ccccc4)ccc3[nH]2)C[C@@H]1C(=O)N[C@H]1C[C@@H](C)N(C#N)C1] | +++ | 528.61 | 529.2 |
| 42 | | | |
| 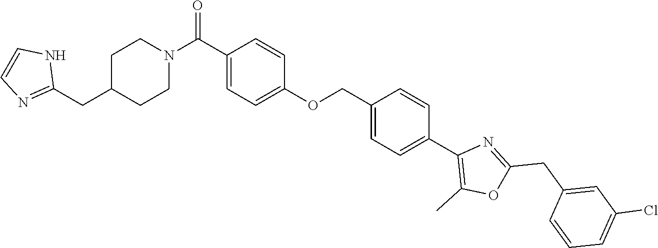 [SMILES: Cc1oc(Cc2cccc(Cl)c2)nc1-c1ccc(COc2ccc(C(=O)N3CCC(Cc4ncc[nH]4)CC3)cc2)cc1] | ++++ | 581.11 | 581.3 |
| 43 | | | |
| 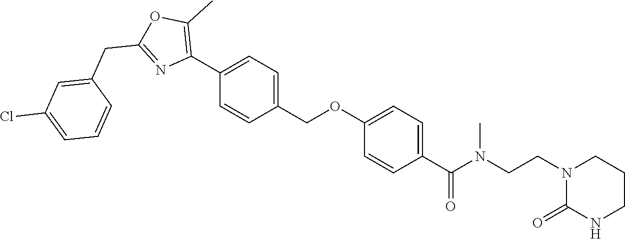 [SMILES: Cc1oc(Cc2cccc(Cl)c2)nc1-c1ccc(COc2ccc(C(=O)N(C)CCN3CCCNC3=O)cc2)cc1] | ++++ | 573.09 | 573.3 |
| 44 | | | |

### Equivalents

The disclosure set forth above may encompass multiple distinct embodiments with independent utility. Although each of these embodiments has been disclosed, the specific embodiments thereof as disclosed and illustrated herein are not to be considered in a limiting sense, because numerous variations are possible. The subject matter of the embodiments includes all novel and nonobvious combinations and subcombinations of the various elements, features, functions, and/or properties disclosed herein. The following claims particularly point out certain combinations and subcombinations regarded as novel and nonobvious. Alternative embodiments as in other combinations and subcombinations of features, functions, elements, and/or properties may be claimed in this application, in applications claiming priority from this application, or in related applications. Such claims, whether directed to a different embodiment or to the same embodiment, and whether broader, narrower, equal, or different in scope in comparison to the original claims, also are regarded as included within the subject matter of this disclosure.

One or more features from any embodiments described herein or in the figures may be combined with one or more features of any other embodiments described herein or in the figures without departing from the scope of this disclosure.

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed is:

1. A compound of Formula (I), Formula (II), or Formula (III): 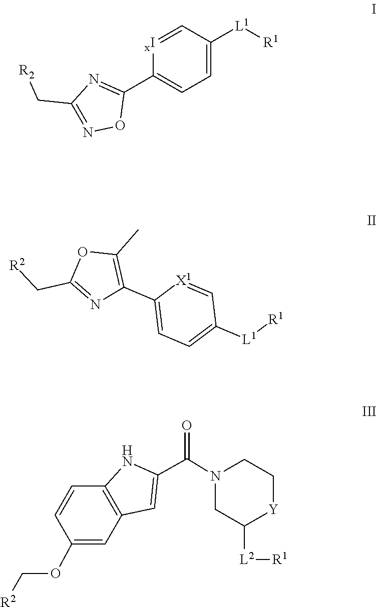
or a pharmaceutically acceptable salt, diastereomer, or stereoisomer thereof;
wherein:
L¹ is selected from 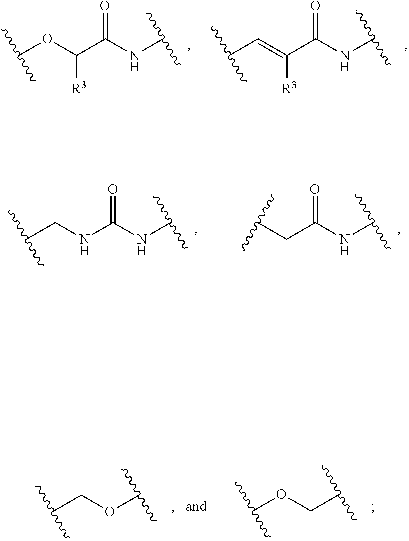
L² is selected from 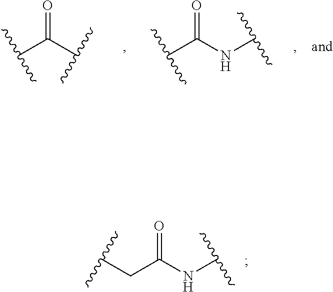
R¹ is selected from aryl substituted with 1, 2, 3, or 4 R^{4a} groups; cycloalkyl substituted with 1, 2, 3, or 4 R^{4a} groups; heteroaryl substituted with 1, 2, 3, or 4 R^{4a} groups; and, heterocycle substituted with 1, 2, 3, or 4 R^{4a} groups wherein the heteroaryl and heterocycle contain at least one nitrogen, oxygen, or sulfur and wherein the nitrogen of the heterocycle is substituted with R^{4b};
R² is selected from aryl optionally substituted with 1, 2, 3, or 4 R⁵ groups; heteroaryl optionally substituted with 1, 2, 3, or 4 R⁵ groups; cycloalkyl optionally substituted with 1, 2, 3, or 4 R⁵ groups; and, heterocycle optionally substituted with 1, 2, 3, or 4 R⁵ groups;
R³ is hydrogen or C₁₋₆alkyl;
each R^{4a} is independently selected from C₁₋₆alkyl, C₂₋₆alkenyl, C₂₋₆alkynyl, C₃₋₆ cycloalkyl, C₁₋₆haloalkyl, C₁₋₆alkoxy, hydroxyC₁₋₆alkyl, aminoC₁₋₆alkyl, C₁₋₆alkylaminoalkyl, C₁₋₆dialkylaminoalkyl, amino, hydroxy, cyano, nitro, halogen, —NR⁶R⁷, —CH₂NR⁶R⁷, —C(O)NR⁶R⁷, —CH₂C(O)NR⁶R⁷, —(CH₂)ₐ—O—(CH₂)_{b}R⁸, and —C(O)R⁹;
each R^{4b} is independently selected from hydrogen, C₁₋₆alkyl, C₃₋₆cycloalkyl, C₁₋₆ haloalkyl, cyano, —CH₂NR⁶R⁷, —C(O)NR⁶R⁷, —CH₂C(O)NR⁶R⁷, —(CH₂)ₐ—O—(CH₂)_{b}R⁸, and —C(O)R⁹;
each R⁵, when present, is independently selected from C₁₋₆alkyl, C₂₋₆alkenyl, C₂₋₆alkynyl, C₃₋₆cycloalkyl, C₁₋₆haloalkyl, C₁₋₆alkoxy, hydroxyC₁₋₆alkyl, aminoC₁₋₆alkyl, C₁₋₆ alkylaminoalkyl, C₁₋₆dialkylaminoalkyl, amino, hydroxy, cyano, nitro, halogen, —NR⁶R⁷, —CH₂NR⁶R⁷, —C(O)NR⁶R⁷, —CH₂C(O)NR⁶R⁷, —(CH₂)ₐ—O—(CH₂)_{b}R⁸, and —C(O)R⁹;
R⁶ and R⁷ are independently selected from hydrogen, C₁₋₆alkyl, C₃₋₆cycloalkyl, aryl, arylC₁₋₆alkyl, heteroaryl, heteroarylC₁₋₆alkyl, heterocycle, and heterocycloC₁₋₆alkyl, wherein R⁶ and R⁷, with the exception of hydrogen, can independently be optionally substituted with 1 or 2 R¹⁰ groups;
or R⁶ and R⁷ are joined together to form a heterocycle or a biheterocycle optionally substituted with 1 or 2 R¹⁰ groups;
R⁸ is hydroxy, cyano, halogen, C₁₋₆haloalkyl, —NR⁶R⁷, or —C(O)R⁹;
R⁹ is C₁₋₆alkyl, hydroxy, C₁₋₆alkoxy, aryl, aryloxy, arylC₁₋₆alkyl, aryloxyC₁₋₆alkyl, heteroaryl, heteroarylC₁₋₆alkyl, heterocycle, heterocycloC₁₋₆alkyl, or —NR¹¹R¹²;
R¹⁰ is independently selected from —C(O)R⁹, —COOH, amino, —NR¹¹R¹², —NR¹¹C(O)R¹², aryl, heteroaryl, arylC₁₋₆alkyl, and heteroarylC₁₋₆alkyl;
or 2 R¹⁰ groups, when on the same carbon, can be taken together to form an oxo group;
R¹¹ and R¹² are independently selected from hydrogen and C₁₋₆alkyl;
R¹⁵ is hydrogen, —C(O)R⁹, C₁₋₆alkyl, or C₃₋₆cycloalkyl;
R¹⁶ and R¹⁷ are independently selected from hydrogen, —C(O)R⁹, C₁₋₆alkyl, and C₃₋₆cycloalkyl; and
Y is NR¹⁵, CR¹⁶R¹⁷, or oxygen;
X¹ is CH or N; and
a and be are integers independently selected from 1, 2, 3, and 4.

2. The compound of claim 1, of Formula (I): 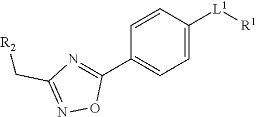
or a pharmaceutically acceptable salt, diastereomer, or stereoisomer thereof.

3. The compound of claim 1, of Formula (II): 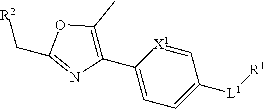
or a pharmaceutically acceptable salt, diastereomer, or stereoisomer thereof.

4. The compound of claim 2 of Formula (Ia) or Formula (Ib): 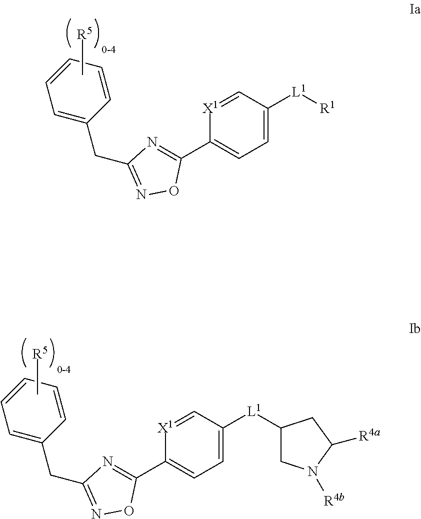
or a pharmaceutically acceptable salt, diastereomer, or stereoisomer thereof.

5. The compound of claim 2 of Formula (Ic) or Formula (Id): 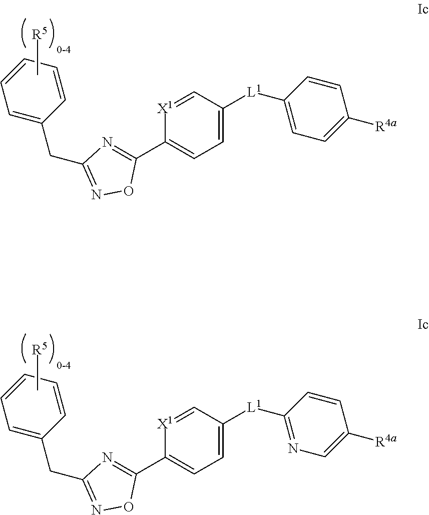
or a pharmaceutically acceptable salt, diastereomer, or stereoisomer thereof.

6. The compound of claim 3 of Formula (IIa) or Formula (IIb): 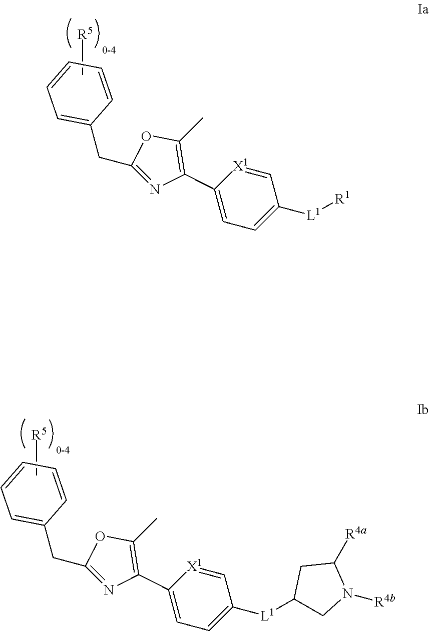
or a pharmaceutically acceptable salt, diastereomer, or stereoisomer thereof.

7. The compound of claim 3 of Formula (IIc) or Formula (IId): 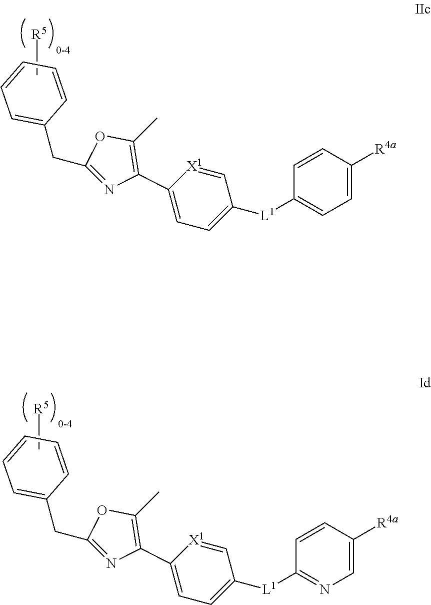
or a pharmaceutically acceptable salt, diastereomer, or stereoisomer thereof.

8. The compound of any one of claims 1-4 and 6, wherein R¹ is aryl substituted with one R^{4a} group.

9. The compound of any one of claims 1-4 and 6, wherein R¹ is heteroaryl substituted with one R^{4a} group.

10. The compound of any one of claims 1-4 and 6, wherein R¹ is heterocycle substituted with one R^{4a} group and wherein the heterocycle contains at least one nitrogen and the nitrogen is substituted with R^{4b}.

11. The compound of any one of claims 1-4, 6, and 8-10, wherein the one R^{4a} group is selected from —NR⁶R⁷, —CH₂NR⁶R⁷, and —C(O)NR⁶R⁷.

12. The compound of claim 11, wherein R⁶ and R⁷ are independently selected from hydrogen, C₁₋₆alkyl, and heterocycloC₁₋₆alkyl.

13. The compound of claim 11, wherein R⁶ and R⁷ are joined together to form a heterocycle optionally substituted with R¹⁰.

14. The compound of claim 13, wherein R¹⁰ is COOH, amino, or NR¹¹R¹².

15. The compound of claim 14, wherein R¹¹ and R¹² are independently selected from hydrogen and C₁₋₆alkyl.

16. The compound of claim 10, wherein R^{4b} is cyano.

17. The compound of claim 10, wherein R^{4b} is hydrogen.

18. The compound of any one of claims 1-3 and 8-17, wherein R² is aryl optionally substituted with one R⁵ group.

19. The compound of any one of claims 1-3 and 8-17, wherein R² is heteroaryl optionally substituted with one R⁵ group.

20. The compound of any one of claims 1-3 and 8-17, wherein R² is heterocycle optionally substituted with one R⁵ group.

21. The compound of any one of claims 18-20 wherein the one R⁵ group is halogen.

22. The compound of claim 21, wherein the halogen is chloro.

23. The compound of any one of claims 18-20, wherein the one R⁵ group is selected from C₁₋₆alkyl, C₁₋₆haloalkyl, C₁₋₆alkoxy, and cyano.

24. The compound of any one of claim 1-23, wherein L¹ is selected from

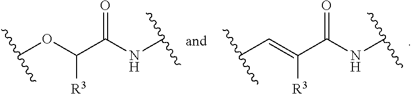

25. The compound of any one of claim 1-23, wherein L¹ is selected from

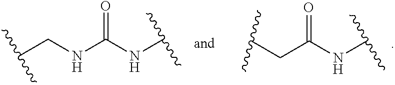 [SMILES: *CNC(=O)N* ; *CC(=O)N*]

26. The compound of any one of claims 1-23, wherein L¹ is selected from

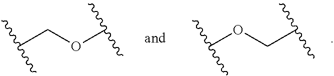

27. The compound of any one of claims 1-26, wherein X¹ is CH.

28. The compound of any one of claims 1-26, wherein X¹ is N.

29. The compound of any one of claim 4-7, wherein R⁵ is chloro.

30. The compound of claim 29, wherein R^{4a} is —C(O)NR⁶R⁷, —NR⁶R⁷, or —CH₂NR⁶R⁷.

31. The compound of claim 29 or 30, wherein R⁶ and R⁷ are independently selected from hydrogen, C₁₋₆alkyl, and heterocycloC₁₋₆alkyl.

32. The compound of claim 29, wherein R^{4a} is C₁₋₆alkyl and R^{4b} is cyano.

33. The compound of claim 32, wherein R^{4a} is methyl.

34. The compound of any one of claims 1-4 and 6, wherein R¹ is selected from: 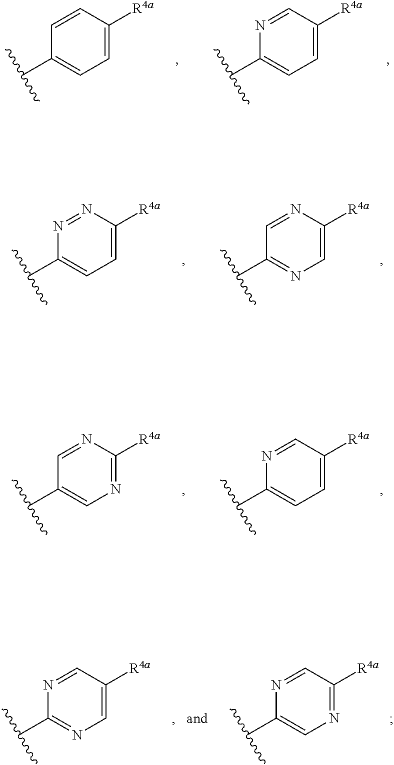
R^{4a} is selected from —C(O)NR⁶R⁷, C₁₋₆alkyl, and —CH₂NR⁶R⁷;
R⁶ and R⁷ are independently selected from hydrogen, C₁₋₆alkyl, and heteroarylC₁₋₆alkyl;
or R⁶ and R⁷ are joined together to form a heterocycle or biheterocycle optionally substituted with R¹⁰; and
R¹⁰ is selected from —COOH, —NH₂, —NHMe, and heteroarylC₁₋₆alkyl.

35. The compound of any one of claims 1-4 and 6, wherein R¹ is selected from

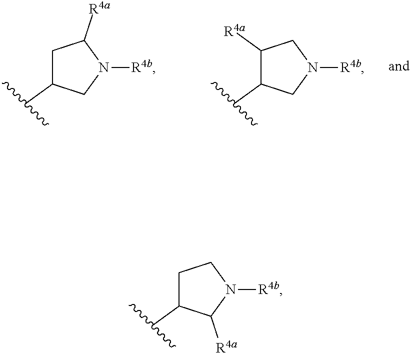

36. The compound of any one of claims 1-4 and 6, wherein R¹ is selected from

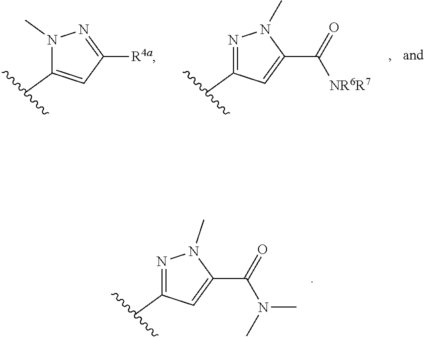

37. The compound of claim 1, wherein L² is

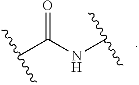 [SMILES: *NC(*)=O]

38. The compound of claim 1 or 37, wherein Y is NR¹⁵.

39. The compound of any one of claims 1 and 37-38, wherein R¹⁵ is —C(O)R⁹.

40. The compound of claim 1, selected from: 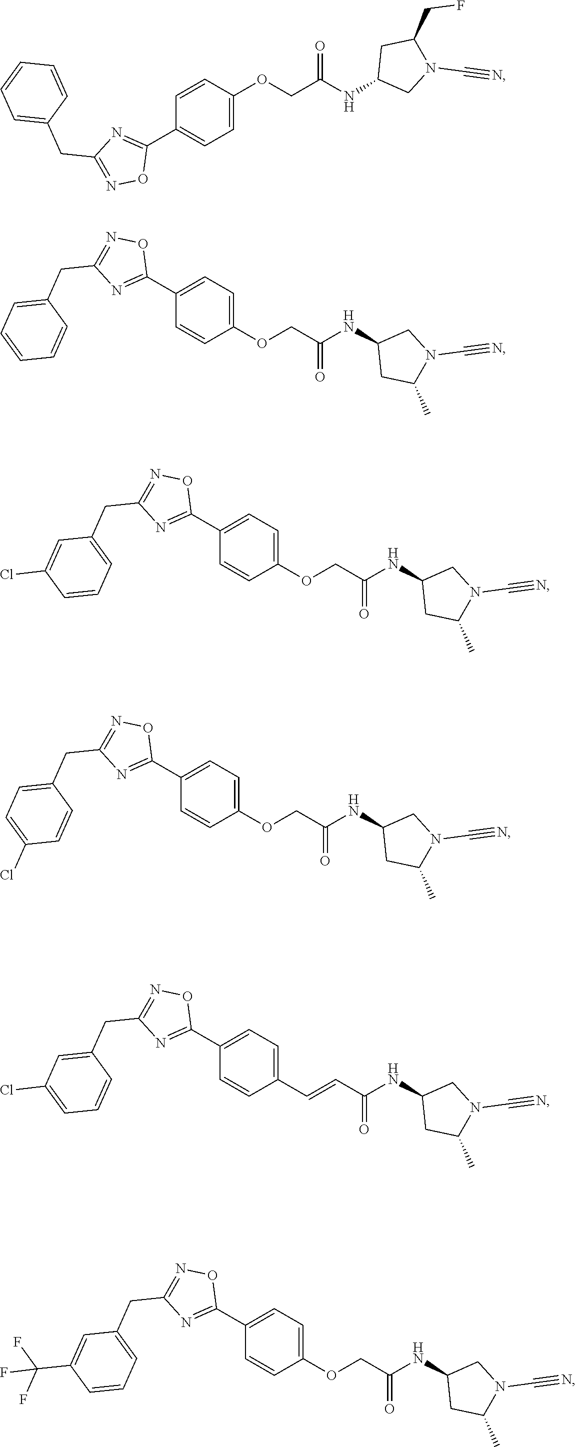 [SMILES: N#CN1C[C@H](NC(=O)COc2ccc(-c3nc(Cc4ccccc4)no3)cc2)C[C@H]1CF ; C[C@@H]1C[C@@H](NC(=O)COc2ccc(-c3nc(Cc4ccccc4)no3)cc2)CN1C#N ; C[C@@H]1C[C@@H](NC(=O)COc2ccc(-c3nc(Cc4cccc(Cl)c4)no3)cc2)CN1C#N ; C[C@@H]1C[C@@H](NC(=O)COc2ccc(-c3nc(Cc4ccc(Cl)cc4)no3)cc2)CN1C#N ; C[C@@H]1C[C@@H](NC(=O)/C=C/c2ccc(-c3nc(Cc4cccc(Cl)c4)no3)cc2)CN1C#N ; C[C@@H]1C[C@@H](NC(=O)COc2ccc(-c3nc(Cc4cccc(C(F)(F)F)c4)no3)cc2)CN1C#N] 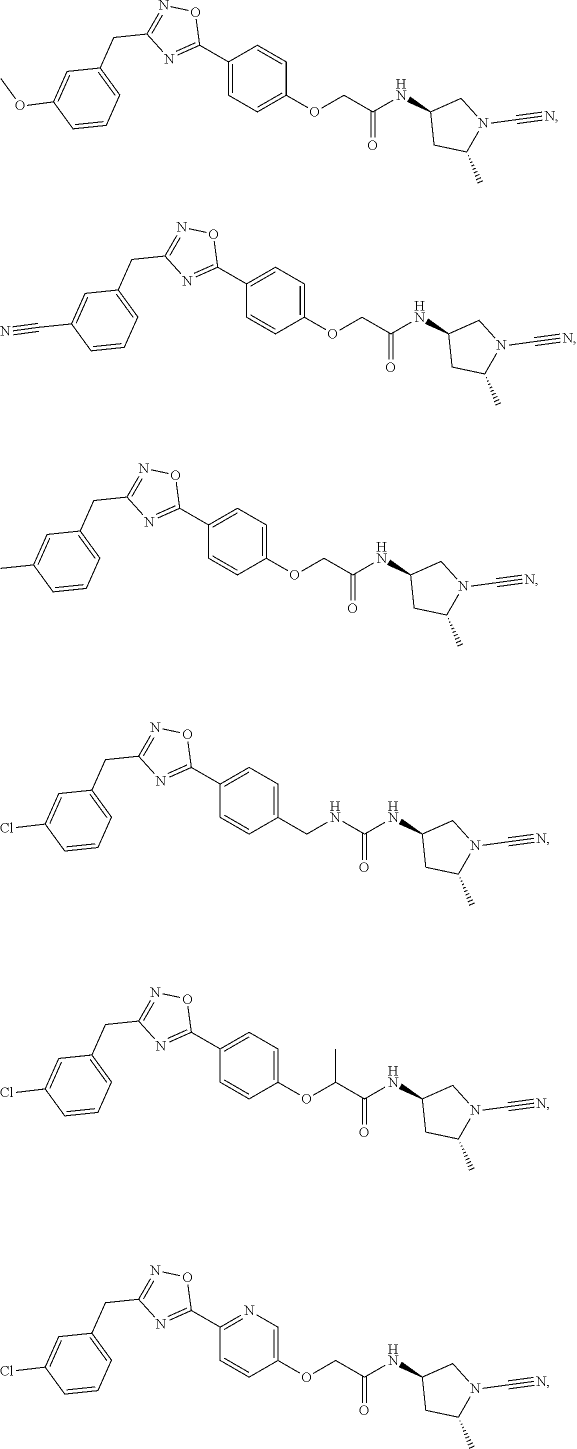 [SMILES: COc1cccc(Cc2noc(-c3ccc(OCC(=O)N[C@@H]4C[C@@H](C)N(C#N)C4)cc3)n2)c1 ; C[C@@H]1C[C@@H](NC(=O)COc2ccc(-c3nc(Cc4cccc(C#N)c4)no3)cc2)CN1C#N ; Cc1cccc(Cc2noc(-c3ccc(OCC(=O)N[C@@H]4C[C@@H](C)N(C#N)C4)cc3)n2)c1 ; C[C@@H]1C[C@@H](NC(=O)NCc2ccc(-c3nc(Cc4cccc(Cl)c4)no3)cc2)CN1C#N ; CC(Oc1ccc(-c2nc(Cc3cccc(Cl)c3)no2)cc1)C(=O)N[C@@H]1C[C@@H](C)N(C#N)C1 ; C[C@@H]1C[C@@H](NC(=O)COc2ccc(-c3nc(Cc4cccc(Cl)c4)no3)nc2)CN1C#N] 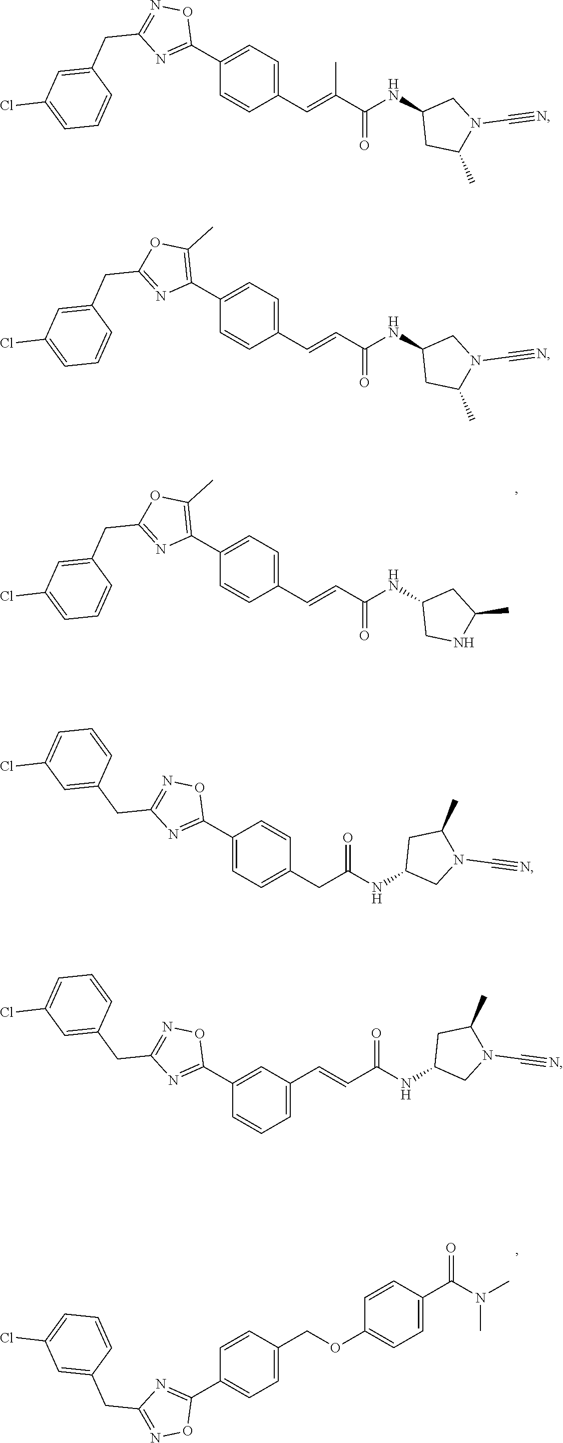 [SMILES: C/C(=C\c1ccc(-c2nc(Cc3cccc(Cl)c3)no2)cc1)C(=O)N[C@@H]1C[C@@H](C)N(C#N)C1 ; Cc1oc(Cc2cccc(Cl)c2)nc1-c1ccc(/C=C/C(=O)N[C@@H]2C[C@@H](C)N(C#N)C2)cc1 ; Cc1oc(Cc2cccc(Cl)c2)nc1-c1ccc(/C=C/C(=O)N[C@H]2CN[C@H](C)C2)cc1 ; C[C@@H]1C[C@@H](NC(=O)Cc2ccc(-c3nc(Cc4cccc(Cl)c4)no3)cc2)CN1C#N ; C[C@@H]1C[C@@H](NC(=O)/C=C/c2cccc(-c3nc(Cc4cccc(Cl)c4)no3)c2)CN1C#N ; CN(C)C(=O)c1ccc(OCc2ccc(-c3nc(Cc4cccc(Cl)c4)no3)cc2)cc1] 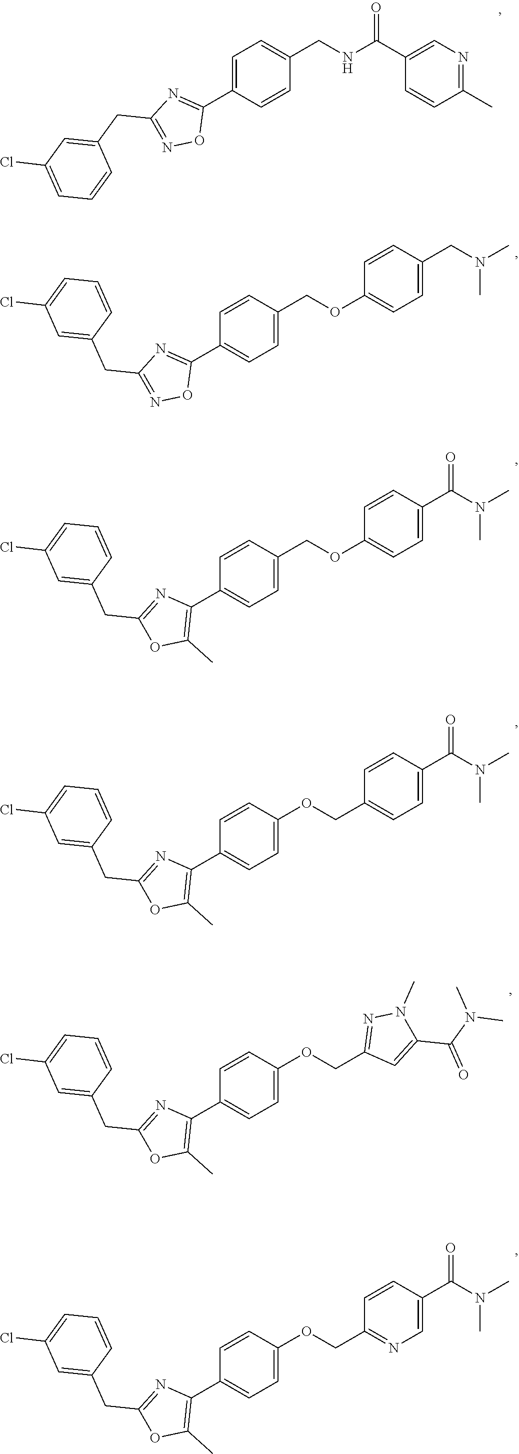 [SMILES: Cc1ccc(C(=O)NCc2ccc(-c3nc(Cc4cccc(Cl)c4)no3)cc2)cn1 ; CN(C)Cc1ccc(OCc2ccc(-c3nc(Cc4cccc(Cl)c4)no3)cc2)cc1 ; Cc1oc(Cc2cccc(Cl)c2)nc1-c1ccc(COc2ccc(C(=O)N(C)C)cc2)cc1 ; Cc1oc(Cc2cccc(Cl)c2)nc1-c1ccc(OCc2ccc(C(=O)N(C)C)cc2)cc1 ; Cc1oc(Cc2cccc(Cl)c2)nc1-c1ccc(OCc2cc(C(=O)N(C)C)n(C)n2)cc1 ; Cc1oc(Cc2cccc(Cl)c2)nc1-c1ccc(OCc2ccc(C(=O)N(C)C)cn2)cc1] 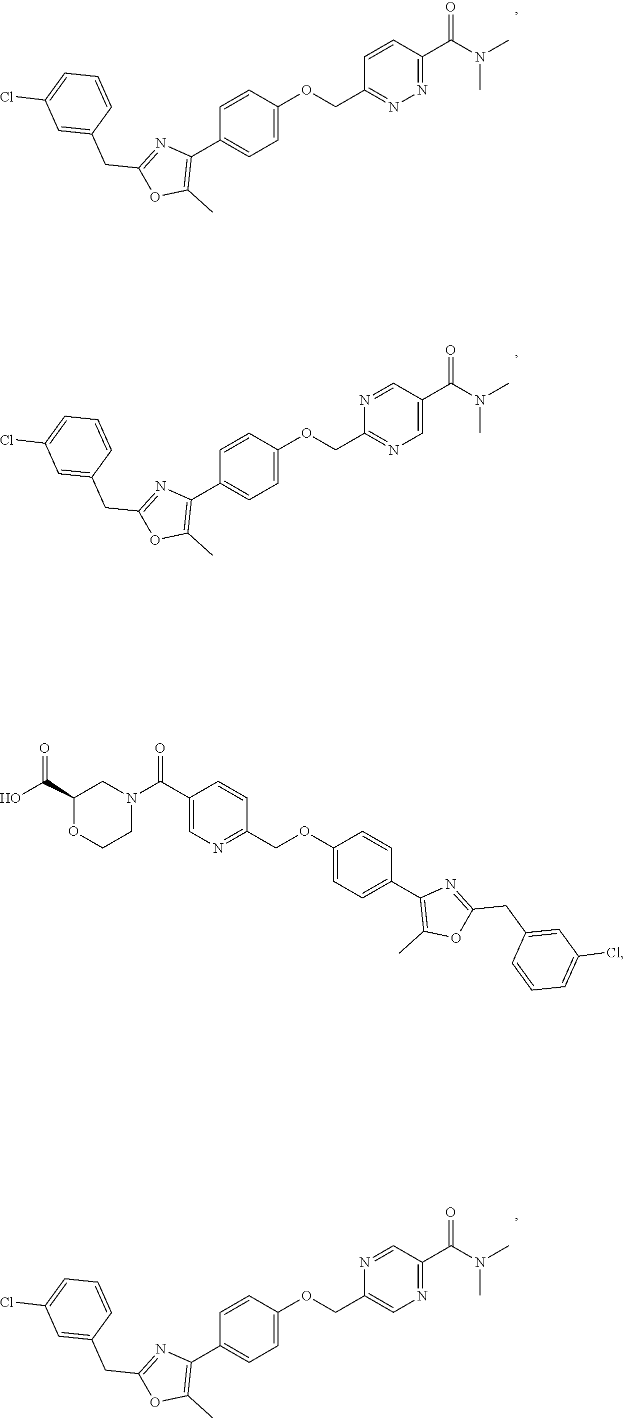 [SMILES: Cc1oc(Cc2cccc(Cl)c2)nc1-c1ccc(OCc2ccc(C(=O)N(C)C)nn2)cc1 ; Cc1oc(Cc2cccc(Cl)c2)nc1-c1ccc(OCc2ncc(C(=O)N(C)C)cn2)cc1 ; Cc1oc(Cc2cccc(Cl)c2)nc1-c1ccc(OCc2ccc(C(=O)N3CCO[C@@H](C(=O)O)C3)cn2)cc1 ; Cc1oc(Cc2cccc(Cl)c2)nc1-c1ccc(OCc2cnc(C(=O)N(C)C)cn2)cc1] 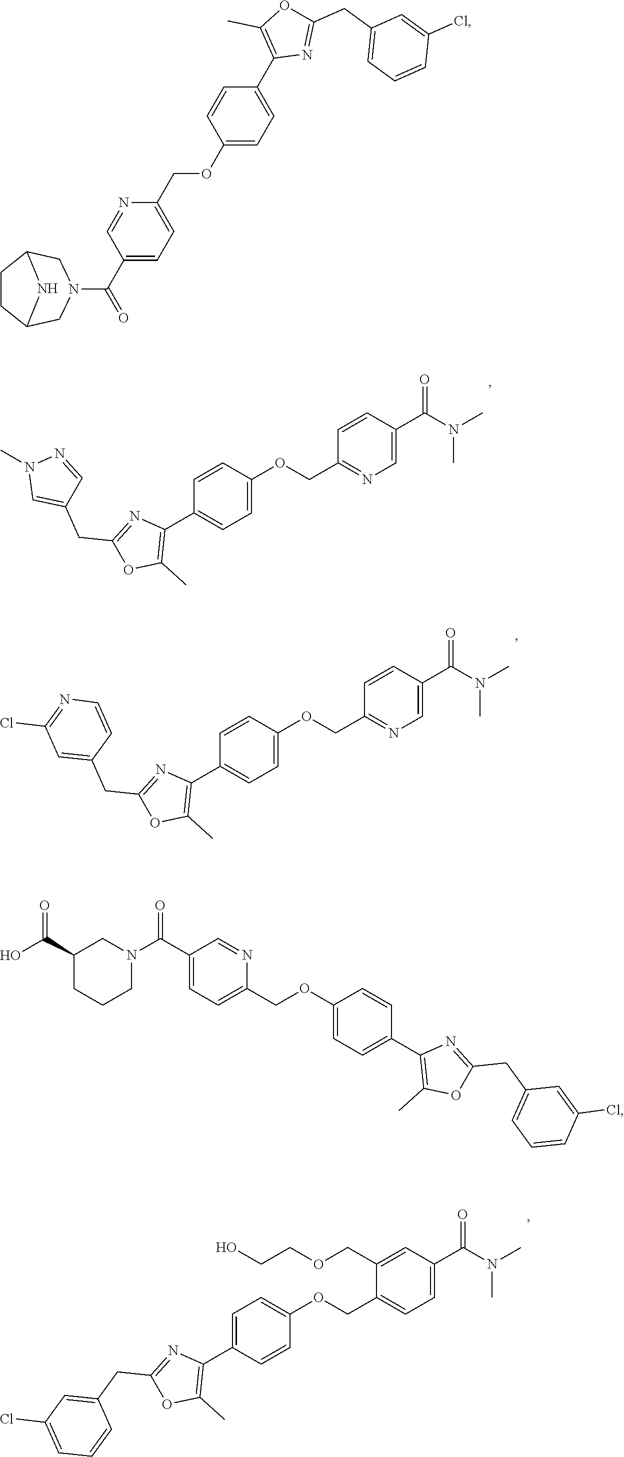 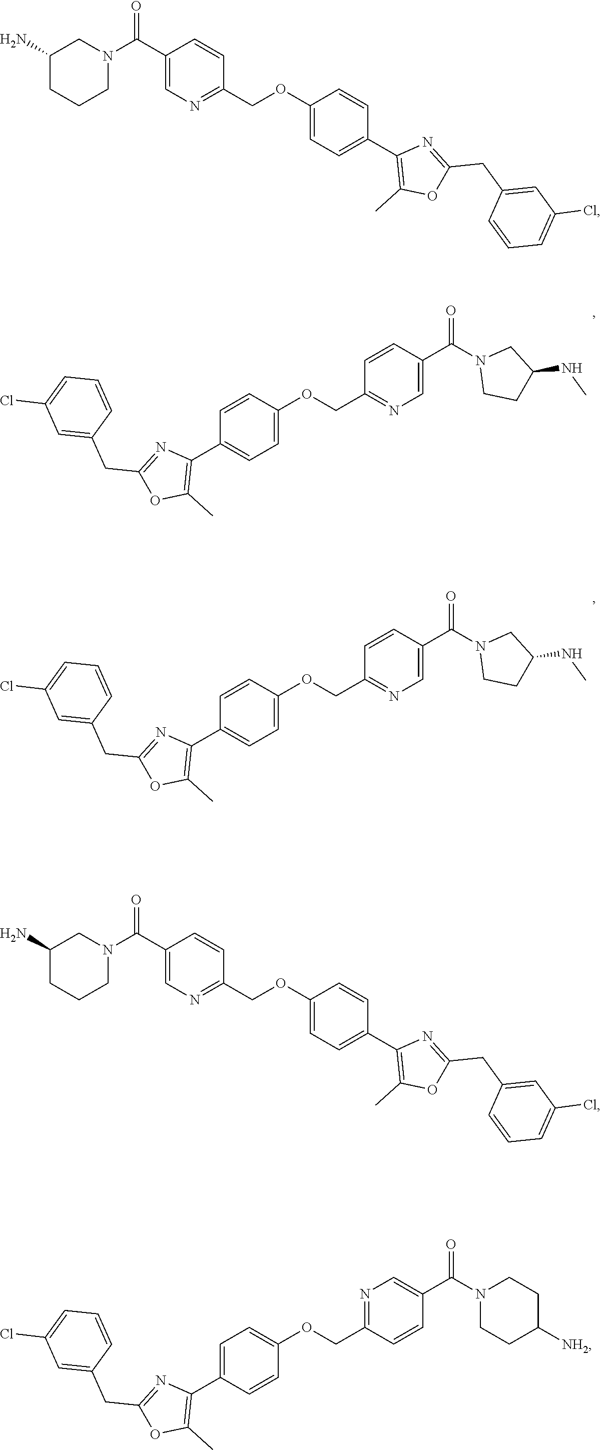 [SMILES: Cc1oc(Cc2cccc(Cl)c2)nc1-c1ccc(OCc2ccc(C(=O)N3CCC[C@H](N)C3)cn2)cc1 ; CN[C@H]1CCN(C(=O)c2ccc(COc3ccc(-c4nc(Cc5cccc(Cl)c5)oc4C)cc3)nc2)C1 ; CN[C@@H]1CCN(C(=O)c2ccc(COc3ccc(-c4nc(Cc5cccc(Cl)c5)oc4C)cc3)nc2)C1 ; Cc1oc(Cc2cccc(Cl)c2)nc1-c1ccc(OCc2ccc(C(=O)N3CCC[C@@H](N)C3)cn2)cc1 ; Cc1oc(Cc2cccc(Cl)c2)nc1-c1ccc(OCc2ccc(C(=O)N3CCC(N)CC3)cn2)cc1] 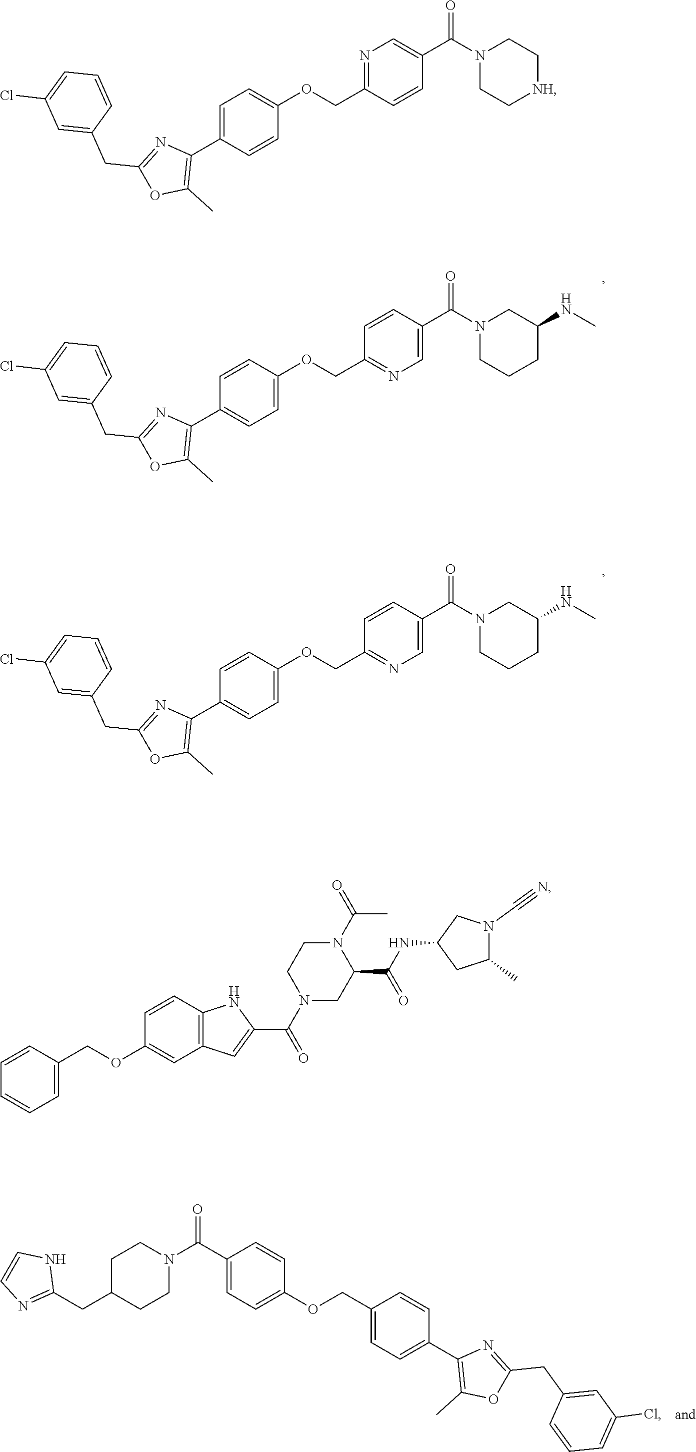 [SMILES: Cc1oc(Cc2cccc(Cl)c2)nc1-c1ccc(OCc2ccc(C(=O)N3CCNCC3)cn2)cc1 ; CN[C@H]1CCCN(C(=O)c2ccc(COc3ccc(-c4nc(Cc5cccc(Cl)c5)oc4C)cc3)nc2)C1 ; CN[C@@H]1CCCN(C(=O)c2ccc(COc3ccc(-c4nc(Cc5cccc(Cl)c5)oc4C)cc3)nc2)C1 ; CC(=O)N1CCN(C(=O)c2cc3cc(OCc4ccccc4)ccc3[nH]2)C[C@@H]1C(=O)N[C@H]1C[C@@H](C)N(C#N)C1 ; Cc1oc(Cc2cccc(Cl)c2)nc1-c1ccc(COc2ccc(C(=O)N3CCC(Cc4ncc[nH]4)CC3)cc2)cc1] 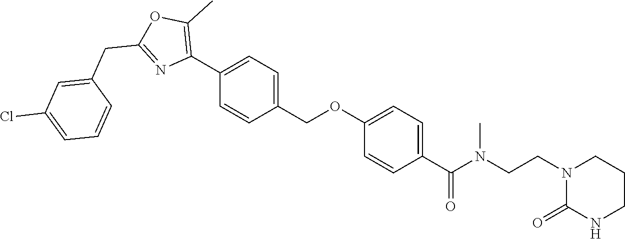 [SMILES: Cc1oc(Cc2cccc(Cl)c2)nc1-c1ccc(COc2ccc(C(=O)N(C)CCN3CCCNC3=O)cc2)cc1]
or a pharmaceutically acceptable salt, diastereomer, or stereoisomer thereof.

41. A pharmaceutical composition comprising the compound of any one of claims 1-40 and a pharmaceutically acceptable carrier, excipient, or diluent.

42. A method of treating a disorder or condition wherein inhibition of USP28 provides therapeutic benefit comprising administering a compound of any one of claims 1-40 or a pharmaceutical composition of claim 41.

43. The method of claim 42, wherein the disease or condition is cancer.

44. The method of claim 42, wherein the cancer is selected from non-small cell lung cancer, breast cancer, intestinal cancer, and bladder cancer.

45. The method of claim 42, wherein the disease or condition is an autoimmune disease, inflammation, or an infectious disease.

46. A compound of any one of claims 1-40 or a pharmaceutical composition of claim 41 for use in the treatment of a disorder or condition wherein inhibition of USP28 provides therapeutic benefit.

47. Use of a compound of one of claims 1-40 or a pharmaceutical composition of claim 41 in the preparation of a medicament for the treatment of a disorder or condition wherein inhibition of USP28 provides therapeutic benefit.
